SEQUENCE LISTING (20 sequences)

SEQ ID NO: 1 - protein, 10 aa, Artificial Sequence
  10C9 Heavy CDR1
DYNFTRYYIH

SEQ ID NO: 2 - protein, 17 aa, Artificial Sequence
  10C9 Heavy CDR2
WIYPGTGDSKYNEKFKG

SEQ ID NO: 3 - protein, 9 aa, Artificial Sequence
  10C9 Heavy CDR3
NGNYWYFDV

SEQ ID NO: 4 - protein, 16 aa, Artificial Sequence
  10C9 Light CDR1
RSSKSLLHSNGNTYLY

SEQ ID NO: 5 - protein, 7 aa, Artificial Sequence
  10C9 Light CDR2
RMSNLAS

SEQ ID NO: 6 - protein, 9 aa, Artificial Sequence
  10C9 Light CDR3
MQHREYPFT

SEQ ID NO: 7 - protein, 10 aa, Artificial Sequence
  10G5 Heavy CDR1
GYSFTDFYIN

SEQ ID NO: 8 - protein, 17 aa, Artificial Sequence
  10G5 Heavy CDR2
RIFPGGDNTYYNEKFKG

SEQ ID NO: 9 - protein, 9 aa, Artificial Sequence
  10G5 Heavy CDR3
RGLYYAMDY

SEQ ID NO: 10 - protein, 16 aa, Artificial Sequence
  10G5 Light CDR1
RSSQSLVHSNGIPYLH

SEQ ID NO: 11 - protein, 7 aa, Artificial Sequence
  10G5 Light CDR2
RVSNRFS

SEQ ID NO: 12 - protein, 9 aa, Artificial Sequence
  10G5 Light CDR3
SQGTHVPPT

SEQ ID NO: 13 - protein, 118 aa, Artificial Sequence
  hu10G5 VH(GH1)
EVQLVQSGAGLVQPGGSVRLSCAASGYSFTDFYINWVRQAPGKGLEWIARIFPGGDNTYYNEKFKGRFTLSADTSSSTAYLQLNSLRAEDTAVYYCARRGLYYAMDYWGQGTLVTVSS

SEQ ID NO: 14 - protein, 118 aa, Artificial Sequence
  hu10G5 VH(GH2)
EVQLVESGGGLVQPGGSLRLSCAASGYSFTDFYINWVRQAPGKGLEWVARIFPGGDNTYYNEKFKGRFTLSADTSKSTAYLQMNSLRAEDTAVYYCARRGLYYAMDYWGQGTLVTVSS

SEQ ID NO: 15 - protein, 112 aa, Artificial Sequence
  hu10G5 VL(GL1)
DIQMTQSPSSLSASVGDRVTITCRSSQSLVHSNGIPYLHWYQQKPGKAPKLLIYRVSNRFSGVPSRFSGSGSGTDFTLTISSLQPEDFATYYCSQGTHVPPTFGQGTKVEIK

SEQ ID NO: 16 - protein, 112 aa, Artificial Sequence
  hu10G5 VL(GL2)
DIQMTQSPSSLSASVGDRVTITCRSSQSLVHSNGIPYLHWYQQKPGKAPKLLIYRVSNRFSGVPSRFSGSRSGTDFTLTISSLQPEDFATYYCSQGTHVPPTFGQGTKVEIK

SEQ ID NO: 17 - protein, 448 aa, Artificial Sequence
  10G5 GH1 Heavy chain
EVQLVQSGAGLVQPGGSVRLSCAASGYSFTDFYINWVRQAPGKGLEWIARIFPGGDNTYYNEKFKGRFTLSADTSSSTAYLQLNSLRAEDTAVYYCARRGLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 448 aa, Artificial Sequence
  10G5 GH2 Heavy chain
EVQLVESGGGLVQPGGSLRLSCAASGYSFTDFYINWVRQAPGKGLEWVARIFPGGDNTYYNEKFKGRFTLSADTSKSTAYLQMNSLRAEDTAVYYCARRGLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 219 aa, Artificial Sequence
  10G5 GL1 Light chain
DIQMTQSPSSLSASVGDRVTITCRSSQSLVHSNGIPYLHWYQQKPGKAPKLLIYRVSNRFSGVPSRFSGSGSGTDFTLTISSLQPEDFATYYCSQGTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, Artificial Sequence
  10G5 GL2 Light chain
DIQMTQSPSSLSASVGDRVTITCRSSQSLVHSNGIPYLHWYQQKPGKAPKLLIYRVSNRFSGVPSRFSGSRSGTDFTLTISSLQPEDFATYYCSQGTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0250169 A1
(43) Pub. Date: Aug. 10, 2023

(54) AXL Inhibitors for Antiviral Therapy

(71) Applicant: BERGENBIO ASA, Bergen (NO)

(72) Inventors: James Bradley LORENS, Bergen (NO); Wendy MAURY, Iowa City, IA (US)

(73) Assignee: BERGENBIO ASA, Bergen (NO)

(21) Appl. No.: 17/995,737

(22) PCT Filed: Apr. 1, 2021
(86) PCT No.: PCT/EP2021/058774

§ 371 Date: Oct. 7, 2022

Related U.S. Application Data
(60) Provisional application No. 63/109,393, filed on Nov. 4, 2020.
(60) Provisional application No. 63/007,019, filed on Apr. 8, 2020.

(51) Int. Cl. C07K 16/28; A61K 31/53; A61K 31/573; C07K 16/24; A61P 31/14

(52) U.S. Cl. CPC C07K 16/28; A61K 31/53; A61K 31/573; C07K 16/248; A61P 31/14

(57) ABSTRACT

This disclosure relates to compostions and methods for preventing and treating a viral infection in a subject. In particular, the present disclosure provides compostions and methods of preventing or treating infection of a subject with a coronavirus such as the Severe Acute Respiratory Syndrome Coronavirus-2 (SARS-CoV-2) that causes the disease COVID-19.

### EARLIER APPLICATIONS

This application claims priority from the following two applications: (1) U.S. provisional application No. 63/007,019, filed 8 Apr. 2020, and (2) U.S. provisional application No. 63/109,393, filed 4 Nov. 2020. Both of priority applications (1) and (2) are hereby incorporated be reference in their entirety and for any and all purposes as if fully set forth herein.

### FIELD

This disclosure relates to compostions and methods for preventing and treating a viral infection in a subject. In particular, the present disclosure provides compostions and methods of preventing or treating infection of a subject with a coronavirus such as the Severe Acute Respiratory Syndrome Coronavirus-2 (SARS-CoV-2) that causes the disease COVID-19.

### BACKGROUND

### RNA Viruses

RNA viruses cause many diseases in wildlife, domestic animals and humans. These viruses are genetically and antigenically diverse, exhibiting broad tissue tropisms and a wide pathogenic potential. The incubation periods of some of the most pathogenic viruses, e.g. the caliciviruses, are very short. Viral replication and expression of virulence factors may overwhelm early defense mechanisms (Xu, W., Revue Scientifique et Technique, Office of international des Epizooties 10:2393-2408 (1991)) and cause acute and severe symptoms.

There are no specific treatment regimes for many viral infections. The infection may be serotype specific and natural immunity is often brief or absent (Murray, R. et al., in: Medical Microbiology (Third Edition) St. Louis Mo., Mosby Press pp. 542-543 (1998)). Immunization against these virulent viruses is impractical because of the diverse serotypes. RNA virus replicative processes lack effective genetic repair mechanisms, and current estimates of RNA virus replicative error rates are such that each genomic replication can be expected to produce one to ten errors, thus generating a high number of variants (Holland, J. in: Emerging Virus, Morse, S. S., Ed., Oxford University Press, New York and Oxford pp. 203-218 (1993)). Often, the serotypes show no cross protection, such that infection with any one serotype does not protect against infection with another. For example, vaccines against the vesivirus genus of the caliciviruses would have to provide protection against over 40 different neutralizing serotypes (Smith, A. et al., Emerg. Jnf Dis. 4: 13-20 (1998)), and vaccines for the other genera of the Caliciviridae are expected to have the same limitations.

Antisense agents have been proposed for treating various types of viral infection. Among the viruses that have been targeted with this class of therapeutic are vesicular stomatitis virus, influenza virus, hepatitis B virus, human papilloma virus, herpes simplex virus, HIV, and foot-and-mouth disease virus (see WO2005/007805).

However, many of the effective antisense strategies employed in cell culture models have not successfully proceeded to clinical trials. The slow progress is due in part to the lack of robust cell culture models. This problem is compounded by the lack of appropriate pre-clinical animal models for the full exploitation of viral gene expression and replication in vivo. The risk in developing antisense antiviral agents without robust culture models and appropriate animal models is great.

Even for thise antisense agents that made it into the clinic (eg. GEM91, ISIS2105, ISIS2922, GEM132, ISIS14803), none offer an effective antiviral therapy against the members of several virus families, including small, single-stranded, positive-sense RNA viruses in the picornavirus, calicivirus, togavirus, coronavirus, and flavivirus families. The emergence of the Sars-CoV-2 in 2019 has created a pressing need to identify new therapeutics effective against the coronavirus family, in particular.

### Coronaviruses

The coronaviruses are enveloped viruses, having a capside having a helical synunetry. They have a single-stranded positive sense RNA genome, and are capable of infecting cells from birds and mammals. The viruses which are members of this very wide family are known to be causative agents for cold (for example hCoV and OC43 viruses), bronchiolitis (for example NL63 virus) or even some forms of several pneumoniae as those observed during the original SARS (Severe Acute Respiratory Syndrome Coronavirus, SARS-CoV) epidemic between 2002 and 2004.

Although they belong to a same viral family, significant differences exist between the different coronaviruses, both at the genetic and structural level, but also in terms of biology and sensitivity to antiviral molecules (see R. Dijkman, L. van der Hoek. J Formos Med Assoc 108 (2009), pp. 270-279; de Wit et al. 2016, Nature Reviews Microbiology. 14, 523-534). Taxalogically, the coronaviruses (family=Coronaviridae) divide into two subfamilies, Letovirinae (1 genus=Alphaletovirus) and Orthocoronavirinae (4 genera=alpha-, beta-, delta-, and gammacoronavirus).

The genus of most note in recent years has been the Betacoronaviruses, which are themselves divided into three lineages: A, B, and C. Two members of the B-lineage (SARS-CoV/SARS and SARS-CoV-2/COVID-19) and one member of the C-lineage (MERS-CoV/MERS) have emerged as novel human pathogens.

Severe Acute Respiratory Syndrome (SARS) was initially described in late 2002 in China's Guangdong province as atypical pneumonia. In general, SARS begins with a fever greater than 38.0° C., with other common symptoms including headache, body aches, and—typically after 2 to 7 days—respiratory symptoms—such as dry cough and trouble breathing.

The primary way that SARS was spread was close person-to-person contact. Many cases of SARS have involved people who cared for or lived with someone with SARS, or had direct contact with infectious material (for example, respiratory secretions) from a person who has SARS. Other potential ways in which SARS can be spread include touching the skin of other people or objects that are contaminated with infectious droplets followed by touching of eye(s), nose, or mouth. This can happen when someone who is carrying SARS coughs or sneezes droplets onto themselves, other people, or nearby surfaces.

These modes of transmission enabled the SARS-CoV virus (family Coronaviridae, genus Betacoronavirus, lineage B) to spread rapidly. By mid-March, 2003 the World Health Organization (WHO) had received reports of more than 150 new suspected cases of unknown origin or cause. By mid April, 2003, over 4400 cases with 263 deaths of patients diagnosed with symptoms of SARS had been documented from 26 different countries, including Canada, China, Hong Kong, Indonesia, Philippines, Singapore, Thailand, Viet Nam and the United States. In total, it is believed the total number of SARS infections was in the region of 8000, with almost 800 deaths. The 29,727 base pair genome sequence of SARS-CoV (Urbani) is available from GenBank at the Web site for the National Center for Biotechnology Information, National Library of Medicine http://www.ncbi.nlm.nih.gov/, accession number ay278741.1.

MERS (Middle-East Respiratory Syndrome) emerged in Saudi Arabia in 2012, with the responsible virus, MERS-CoV, belonging to the family Coronaviridae, genus Betacoronavirus, lineage C. Although most cases of MERS-CoV in humans are attributable to a human-to-human transmission, camels appear to be a permanent MERS-CoV infected intermediate animal host and thus make up the main infection animal source in humans.

Approximately 200 cases of MERS infection have ben reported, with a mortality rate of ˜35% (WHO statistics). Many of the reported symptoms are similar to SARS, with fever (98%), cough (83%), shortness of breath (72%), myalgia (32%), diarrhea (26%), and vomiting (21%) all commonly reported. Like SARS, MERS can range from asymptomatic disease to severe pneumonia leading to acute respiratory distress syndrome (ARDS) (see Assiri A et al. 2013, The Lancet. Infectious Diseases. 13 (9): 752-61). The number of MERS cases reported in 2019 was just over 200.

### SARS-CoV-2 and COVID-19

Severe acute respiratory syndrome coronavirus 2 (SARS-CoV-2) is a positive-sense single-stranded RNA virus (family Coronaviridae, genus Betacoronavirus, lineage B). It causes coronavirus disease 2019 (COVID-19), a respiratory illness with symptoms similar to those reported for SARS and MERS.

SARS-CoV-2n was first discovered in Wuhan, China, in late 2019. It is believed to have zoonotic origins and has close genetic similarity to bat coronaviruses, suggesting it emerged from a bat-borne virus, potentially with an intermediate animal reservoir such as a pangolin, prior to making the leap into humans (see Benvenuto D., et al, 2020, Journal of Medical Virology. 92 (4): 455-459. doi:10.1002/jmv.25688).

SARS-CoV-2 is highly contagious in humans, with the World Health Organization (WHO) designated the ongoing 2019/2020 outbreak of COVID-19 as a pandemic on 11 Mar. 2020. As of 8 Apr. 2020, there had been almost 1.5 million reported cases of COVID-19 worldwide with over 80,000 deaths. By 4 Nov. 2020, the worldwide total of reported cases had reached approximately 47 million, with approximately 1.2 million deaths. By 31 Mar. 2021, the worldwide total of reported cases had reached approximately 128 million, with approximately 2.8 million deaths. Like SARS-CoV, the virus is primarily spread between people through close contact and via respiratory droplets produced from coughs or sneezes. Early reports indicate that the virus enters human cells by binding to the receptor angiotensin converting enzyme 2 (ACE2) (see Hoffman M, et al. 2020, Cell. 181: 1-10. doi:10.1016/j.cell.2020.02.052).

The 29,903 base pair genome sequence of SARS-CoV-2 (Wuhan-Hu-1) is available from GenBank at the Web site for the National Center for Biotechnology Information, National Library of Medicine http://www.ncbi.nlm.nih.gov/, accession number MN908947, version number MN908947.3.

In the time since the genome sequence of the Wuhan-Hu-1 strain of SARS-CoV-2 was published multiple other SARS-CoV-2 variants have been identified and their genome sequences published. In particular, several independent lineages of SARS-CoV-2 of particular interest have been reported: UK, B.1.1.7; South Africa, B.1.351; US, B.1.526; and Brazil, P.1 (Zhou et al., Cell 189, 1-14, Apr. 29, 2021). These variants have multiple changes in the immunodominant spike protein that facilitates viral cell entry via the angiotensin-converting enzyme-2 (ACE2) receptor. Mutations in the receptor recognition site on the spike are of concern for their potential for immune escape, with initial reports of reduced protective efficacy against—for example—the B.1.351 variant of first-generation vaccines whose design was based on the initial Wuhan-Hu-1 sequence (see Mahase M, BMJ 2021; 372:n597).

Structural analysis of the variants of concern have identified several common point mutations that appear to confer degrees of immune escape and/or increased infectivity. Several reports identify the E484K mutation as the principal driver of immune escape, with this mutation identified in each of the B.1.351, B.1.526, and P.1 variants; E484K has also been identified in several sub-variants of the B.1.1.7 variant (Wise J, BMJ 2021; 372:n359). Other mutations of note include K417N/T and N501Y which appear to act together to evade some antibody classes. The N501Y mutation is also of note as a main driver of tighter ACE2 binding and, consequently it is believed, increased injectivity (Zhou et al., Cell 189, 1-14, Apr. 29, 2021). Additionally, both B.1.1.7 and P1 share the same 11288:9 deletion (Darby A, BMJ 2021; 372:n771).

Wilfredo F. Garcia-Beltran et al. have reported a detailed comparison of the ability of various Sars-CoV-2 variants to escape humoral immunity (see “Multiple SARS-CoV-2 variants escape neutralization by vaccine-induced humoral immunity, Cell, 2021, ISSN 0092-8674, https://doi.org/10.1016j.cell.2021.03.013.). The B.1.351 variant is reported as being least effectively neutralised by antibody sera raised against the original Wuhan-Hu-1 strain, with the majority of the immune escape ability conferred by the three receptor-binding-domain (RBD) mutations: E484K, N501Y, and K417N/T. In contrast, an engineered variant comprising all of the non-RBD mutations of B.1.351 (L18F, ΔL242-244, D80A, D215G, D614G, and A701V) but not the three RBD mutations had only slight immune escape ability.

The significant and growing public health toll of COVID-19 has created a pressing need for the identification and validation of suitable treatments, in particular treatments able to effectively tackle emerging SARS-CoV-2 variants. Adding to this need is the imperative of minimizing the already vast and increasing economic damage being caused to the world economy by the strict lockdown and social distancing measures implemented by many national governments in their efforts to slow the rate of SARS-CoV-2 transmission.

### SUMMARY

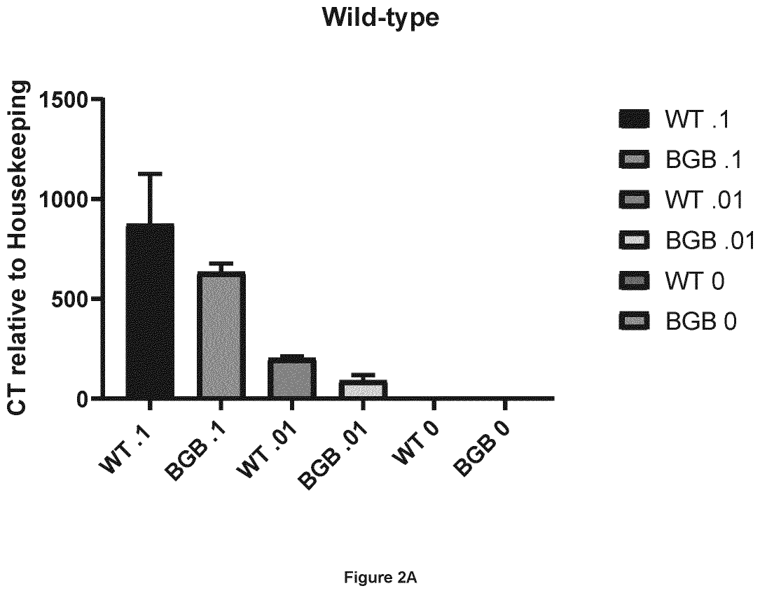

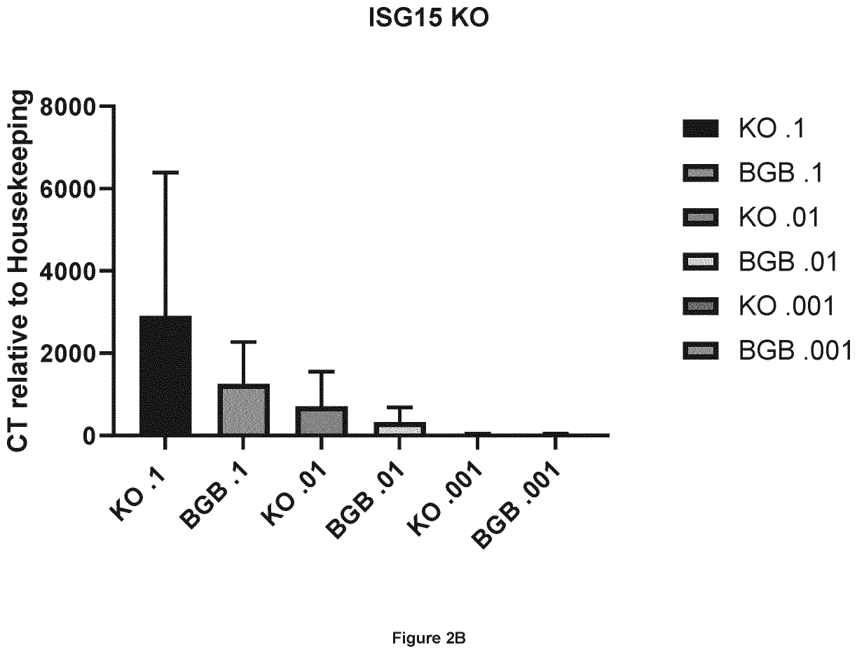

The present authors conducted a preliminary analysis of bemcentinib in a mouse betacoronavirus model system (mouse hepatitis virus, MHV). MHV was used to infect in primary murine bone marrow-derived macrophages (BMDM). As shown in FIG. 2, preliminary results show that bemcentinib treatment reduces coronavirus load in cells at 20 h following infection.

Notably, viral-induced syncytia formation, characteristic of coronaviruses, experimentally and clinically, was reduced in the bemcentinib-treated cell cultures.

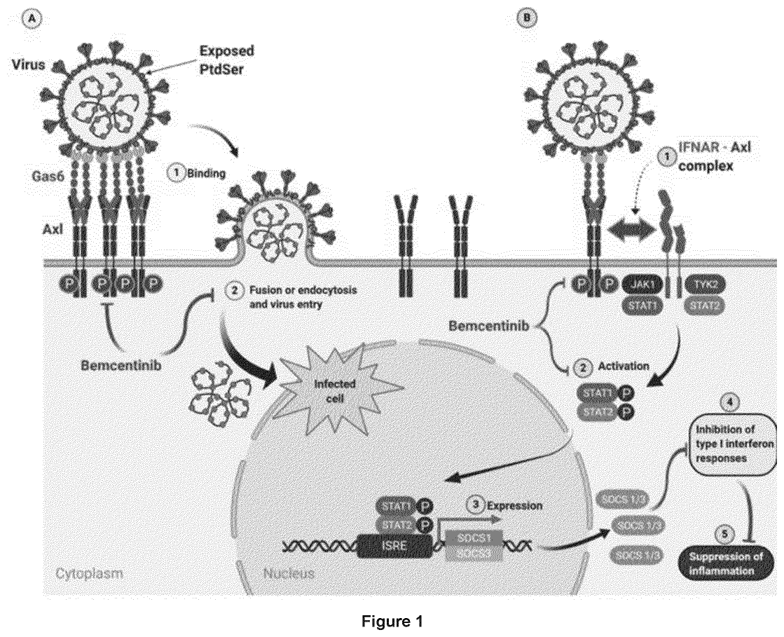

Further, BMDM from mice lacking the type 1 interferon response gene ISG15, a ubiquitin-like protein with potent antiviral activity was also evaluated. ISG15 is one of several IFN-stimulated genes shown to be elevated by bemcentinib treatment (present authors unpublished results). The inhibitory effect of bemcentinib on virus infection was more variable and in general reduced in BMDM from ISG15-null and a mouse strain carrying an inactive ISG15 deconjugase (USP18C61A/C61A; Zhang Y et al. 209, Nat Commun. 10:5383), consistent with the proposed AXL-mediated mechanisms outlined in FIG. 1. These results indicates that bemcentinib has potential to treat and/or prevent SARS-CoV-2 infection.

Recent results highlight that SARS-CoV-2 shows a significant level of entry into cells independent of the human ACE-2 protein, the reported SARS-CoV-2 spike protein receptor (Hoffmann M et al. 2020, Cell. 181, pp. 1-10; https://doi.org/10.1016/j.cell.2020.02.052). This expanded SARS-CoV-2 tropism is likely to include PS-dependent viral uptake and target critical immune cell populations (e.g. macrophages, dendritic cells) that produce IFN and mobilize anti-viral immunity (FIG. 1).

Importantly delayed IFN signaling is characteristic of pathogenic human betacoronaviruses and correlates with disease severity in animal models, suggesting that early intervention with IFN-activating treatment will provide optimal therapeutic benefit (Channappanavar et al. 2016, Cell 19:181).

From these observations, the present authors reasoned that inhibiting the activity of the AXL kinase would act to attenuate SARS-CoV-2 pathogenesis both by limiting viral uptake and promoting anti-viral immunity. In particular, bemcentinib offers immediate hope in the setting of populations at risk (elderly or comorbid individuals typically in protective self-isolation) and those with early infection. Use of bemcentinib in these populations offers an opportunity to explore a safe, potent, easily administered inhibitor of the AXL receptor for prophylaxis and early intervention of SARS-CoV-2 infection.

Accordingly, in a first aspect the present disclosure provides a method for treating a virus infection in a subject, the method comprising administering to the subject an effective amount of an inhibitor of AXL activity or expression (AXLi).

Also provided is a method for preventing or reducing transmission of a virus infection, the method comprising administering to the subject an effective amount of an inhibitor of AXL activity or expression (AXLi).

Preferably, the virus infection is a coronavirus infection. For example, in some case the virus infection is an alphaletovirus infection. In other cases, the virus infection is an orthocoronavirus infection, such as an alphacoronavirus infection, betacoronavirus infection, gammacoronavirus infection, or deltacoronavirus infection.

In preferred embodiments, the viral infection is a betacoronavirus infection, with lineage B infection particularly preferred. For example, in some embodiments the virus infection is a SARS-CoV infection. Most preferably, the virus infection is a SARS-CoV-2 infection.

In other embodiments, the virus infection is a betacoronavirus, lineage C, infection. In some embodiments, the virus infection is a MERS-CoV infection.

In some embodiments, the AXLi is administered in combination with a second antiviral agent. The AXLi may be administered before, after, or simultaneous with the second antiviral agent. In some cases the second antiviral agent is selected from the group consisting of: a protease inhibitor, a helicase inhibitor, and a cell entry inhibitor. In some cases the second antiviral agent is remdesivir.

In some embodiments, the AXLi is administered in combination with an anti-inflammatory agent. The anti-inflammatory agent may be corticosteroid or a glucocorticoid steroid such as dexamethasone.

In some embodiments, the AXLi is administered in combination with an immunosuppressive agent. The immunosuppressive agent may be an IL-6 antagonist such as Tocilizumab.

In preferred embodiments the subject is human. In some cases the subject has, is suspected of having, or is at high risk of having a viral infection. In some embodiments the subject is a healthcare professional.

In some embodiments the subject is at risk of severe symptoms if they were to catch the viral infection. In some cases the subject has one or more comorbidity selected from: respiratory system disease, cardiovascular disease, diabetes, hypertension, cancer, or a suppressed immune system.

In some embodiments the subject is at least 60 years old, such as at least 70, or at least 80 years old. In some cases the subject is male.

The AXLi may be a compound of formula (I): as described in more detail elsewhere herein.

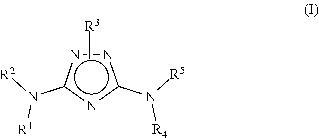

In preferred embodiments the AXLi is bemcentinib. The AXLi may also be an antibody; for example, an antibody comprising the 6 CDRs having the sequences of SEQ ID Nos. 1 to 6, or the 6 CDRs having the sequences of SEQ ID Nos. 7 to 12.

### DETAILED DESCRIPTION

The present authors have conducted a preliminary analysis of bemcentinib in a coronavirus model system, where positive indications of becentinb efficacy are consistent with initial reports of mild Sars-CoV-2 infection in a bemcentinib-dosed human subject. Building on these experiential observations using their knowledge of Axl biology and bemcentinib action, the authors reasoned that inhibiting the activity of the AXL kinase would act to attenuate SARS-CoV-2 pathogenesis in humans both by limiting viral uptake and promoting anti-viral immunity.

Accordingly, in a first aspect the present disclosure provides a method for treating a virus infection in a subject, the method comprising administering to the subject an effective amount of an inhibitor of AXL activity or expression (AXLi).

### AXL

All of the protein kinases that have been identified to date in the human genome share a highly conserved catalytic domain of around 300 amino acids. This domain folds into a bi-lobed structure in which resides ATP-binding and catalytic sites. The complexity of protein kinase regulation allows many potential mechanisms of inhibition including competition with activating ligands, modulation of positive and negative regulators, interference with protein dimerization, and allosteric or competitive inhibition at the substrate or ATP binding sites.

AXL (also known as UFO, ARK, and Tyro7; nucleotide accession numbers NM_021913 and NM_001699; protein accession numbers NP_068713 and NP_001690) is a receptor protein tyrosine kinase (RTK) that comprises a C-terminal extracellular ligand binding domain and N-terminal cytoplasmic region containing the catalytic domain. The extracellular domain of AXL has a unique structure that juxtaposes immunoglobulin and fibronectin Type III repeats and is reminiscent of the structure of neural cell adhesion molecules. AXL and its two close relatives, Mer/Nyk and Sky (Tyro3/Rse/Dtk), collectively known as the ‘TAM’ or Tyro3 family of RTK's, all bind and are stimulated to varying degrees by the same ligand, GAS6 (growth arrest specific-6), a ˜76 kDa secreted protein with significant homology to the coagulation cascade regulator, Protein S. In addition to binding to ligands, the AXL extracellular domain has been shown to undergo homophilic interactions that mediate cell aggregation, suggesting that one important function of AXL may be to mediate cell-cell adhesion.

AXL is predominantly expressed in the vasculature in both endothelial cells (EC's) and vascular smooth muscle cells (VSMC's) and in cells of the myeloid lineage and is also detected in breast epithelial cells, chondrocytes, Sertoli cells and neurons. Several functions including protection from apoptosis induced by serum starvation, TNF-α or the viral protein E1A, as well as migration and cell differentiation have been ascribed to AXL signalling in cell culture. However, Axl−/− mice exhibit no overt developmental phenotype and the physiological function of AXL in vivo is not clearly established in the literature.

### AXL Pathology

The overexpression of AXL and/or its ligand has also been reported in a wide variety of solid tumor types including, but not limited to, breast, renal, endometrial, ovarian, thyroid, non-small cell lung carcinoma, and uveal melanoma as well as in myeloid leukemias. Furthermore, it possesses transforming activity in NIH3T3 and 32D cells. It has been demonstrated that loss of Axl expression in tumor cells blocks the growth of solid human neoplasms in an in vivo MDA-MB-231 breast carcinoma xenograft model. Taken together, these data suggest AXL signalling can independently regulate EC angiogenesis and tumor growth and thus represents a novel target class for tumor therapeutic development.

The expression of AXL and GAS6 proteins is upregulated in a variety of other disease states including endometriosis, vascular injury and kidney disease and AXL signalling is functionally implicated in the latter two indications. AXL-GAS6 signalling amplifies platelet responses and is implicated in thrombus formation. AXL may thus potentially represent a therapeutic target for a number of diverse pathological conditions including solid tumors, including, but not limited to, breast, renal, endometrial, ovarian, thyroid, non-small cell lung carcinoma and uveal melanoma; liquid tumors, including but not limited to, leukemias (particularly myeloid leukemias) and lymphomas; endometriosis, vascular disease/injury (including but not limited to restenosis, atherosclerosis and thrombosis), psoriasis; visual impairment due to macular degeneration; diabetic retinopathy and retinopathy of prematurity; kidney disease (including but not limited to glomerulonephritis, diabetic nephropathy and renal transplant rejection), rheumatoid arthritis; osteoporosis, osteoarthritis and cataracts.

### TAM Receptor Family in Viral Infection

The TAM receptor family, of which AXL is a member, has been implicated in promoting the infective process of a number of enveloped viruses including pox-, retro-, flavi-, arena-, filo-, and alpha-viruses (Shimojima M et al. 2006, J Virol. 80:10109 // Brindley M A et al. 2011, Virology 415:83 // Meertens L et al. 2012, Cell Host & Microbe, 12:544 // Dowall S D et al. 2016, Viruses, 8:27 // Meertens L et al. 2017, Cell Rep 18:324). In these cases TAM activity is believed to increase viral infection through two mechanisms: 1) enhanced viral entry through “apoptotic mimicry”; and 2) suppression of anti-viral type I interferon (IFN) responses (see FIG. 1).

TAM activity is thought to be important for the clearance of apoptotic cells (efferocytosis) by macrophages (Lemke G. 2019, Nature Reviews Immunology, 19: 539), a process often co-opted by enveloped viruses to expand tropism and enhance viral entry. This mimicry does not involve a direct interaction of TAM receptor with virus but rather an interaction between TAM receptor and virions that are opsonized with a TAM ligand (Meertens et al., 2012 ibid); typically in vivo that ligand is Protein S as this is present at ˜300 nM in the vertebrate bloodstream, but a similar system has been posited for Axl and its ligand, Gas6 (Bhattacharyya S et al. 2013; Cell Host Microbe 14:136).

It has been reported that the binding of the viral particle to GAS6-AXL activates signal transduction through Axl's tyrosine kinase domain to suppress type I interferon (IFN) signaling and thus facilitate viral replication (Bhattacharyya 2013 ibid. // Meertens L et al. 2017, Cell Rep 18:324). Consistent with this report, modulation of innate immune responses by Axl, in particular viral-induced IFN responses via SOCS1/3, has been implicated in increased viral replication in infected cells and decreased anti-viral defenses of neighboring cells in both Hepatitis B and Zika infections (Huang M T et al. 2015, Eur. J. Immunol. 45:1696 // Chen J et al. 2018, Nat Microbiol 3:302 // Strange D P et al. 2019, mBio 10:e01372). Loss of the Axl receptor ameliorates severe zika virus pathogenesis and reduces apoptosis in microglia, suggesting a possible role for AXL inhibitors as Zika therapeutics (Hastings et al. 2019, iScience 13:339).

Therapeutic AXL receptor inhibition ameliorated pulmonary pathology resulting from primary viral infection by respiratory syncytial virus (RSV) and H1N1 influenza. Specifically, during primary respiratory syncytial virus (RSV) infection, AXL inhibition increased the number of IFNg-producing T cells and NK cells, suppressed RSV replication and whole lung levels of IL-4 and IL-13. Against H1N1 in mice, AXL inhibition reduced the lethal effect of intrapulmonary infection inflammation, suppressed neutrophil infiltration, and increased the number of IFN-b-producing macrophages and dendritic cells (Shibata T et al. 2014, J Immunology, 192: 3569).

Finally, the Axl inhibitor bemcentinib was one of sixty compounds evaluated by Public Health England as an experimental therapy for Ebola virus using its Biosaftey Containment Level 4 facilities at Porton Down. Bemcentinib was one of only two compounds to show some protective/therapeutic effect against Ebola infection in animal models (Dowall S D et al. 2016, Viruses 2016, 8:27).

### AXL Inhibitors

In view of the role played by AXL in numerous pathological conditions, the development of safe and effective AXL inhibitors has been a topic of interest in recent years. Different groups of AXL inhibitors are discussed in, inter alia, US20070213375, US 20080153815, US20080188454, US20080176847, US20080188455, US20080182862, US20080188474, US20080117789, US20090111816, WO2007/0030680, WO2008/045978, WO2008/083353, WO2008/0083357, WO2008/083354, WO2008/083356, WO2008/080134, WO2009/054864, and WO/2008/083367.

### Small Molecule AXL Inhibitors

### General Formula

In some embodiments the AXL inhibitor is a compound of formula (I):

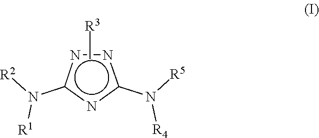

wherein:
R¹, R⁴ and R⁵ are each independently selected from the group consisting of hydrogen, alkyl, alkenyl, aryl, aralkyl, —C(O)R⁸, —C(O)N(R⁶)R⁷, and —C(═NR⁶)N(R⁶)R⁷;
R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR⁸, —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
or R² is a polycyclic heteroaryl containing more than 14 ring atoms as described above and R³ is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
or R³ is a polycyclic heteroaryl containing more than 14 ring atoms as described above, and R² is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
each R⁶ and R⁷ is independently selected from the group consisting of hydrogen, alkyl, alkenyl, alkynyl, haloalkyl, haloalkenyl, haloalkynyl, hydroxyalkyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, haloalkenyl, haloalkynyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, and optionally substituted heteroarylalkynyl;
each R⁹ is independently selected from the group consisting of a direct bond, an optionally substituted straight or branched alkylene chain, an optionally substituted straight or branched alkenylene chain and an optionally substituted straight or branched alkynylene chain;
each R¹⁰ is independently selected from the group consisting of an optionally substituted straight or branched alkylene chain, an optionally substituted straight or branched alkenylene chain and an optionally substituted straight or branched alkynylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or two R^{12's}, together with the common nitrogen to which they are both attached, form an optionally substituted N-heterocyclyl or an optionally substituted N-heteroaryl;
each R¹³ is independently selected from the group consisting of a direct bond, an optionally substituted straight or branched alkylene chain and an optionally substituted straight or branched alkenylene chain; and
each R¹⁴ is independently selected from the group consisting of an optionally substituted straight or branched alkylene chain and an optionally substituted straight or branched alkenylene chain;
as an isolated stereoisomer or mixture thereof or as a tautomer or mixture thereof, or a pharmaceutically acceptable salt or N-oxide thereof.

### Some Embodiments

In some embodiments of the use of the present disclosure, the compound of formula (I) is a compound of formula (Ia):

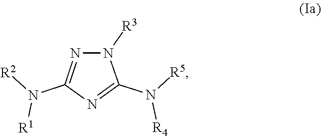

wherein R¹, R², R³, R⁴ and R⁵ are as described above for compounds of formula (I), as an isolated stereoisomer or mixture thereof or as a tautomer or mixture thereof, or a pharmaceutically acceptable salt or N-oxide thereof.

In some embodiments in the compound of formula (Ia) as set forth above, R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and R¹, R⁴, R⁵, each R⁶, each R⁷, each R⁸, each R⁹, each R¹⁰, each R¹¹ and R¹² are as described above for compounds of formula (Ia).

Another embodiment is the use where, in the compound of formula (Ia) as set forth above:

R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; each R⁹ is independently selected from the group consisting of a direct bond and an optionally substituted straight or branched alkylene chain;
each R¹⁰ is an optionally substituted straight or branched alkylene chain; and
each R¹¹ is independently selected from the group consisting of hydrogen, alkyl, cyano, nitro and —OR⁸.

In some embodiments the compound of formula (Ia) as set forth above:

R² and R³ are each independently a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁸)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ia) is 1-(6,7-dihydro-5H-benzo[6,7]cyclohepta[1,2-c]pyridazin-3-yl)-N³-(5′,5′-dimethyl-6,8,9,10-tetrahydro-5H-spiro[cycloocta[b]pyridine-7,2′-[1,3]dioxane]-3-yl)-1H-1,2,4-triazole-3,5-diamine.

In some embodiments in the compound of formula (Ia) as set forth above, R² is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); R³ is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and R¹, R⁴, R⁵, each R⁶, each R⁷, each R⁸, each R⁹, each R¹⁰, each R¹¹, each R¹², each R¹³ and each R¹⁴ are as described above for compounds of formula (Ia).

In some embodiments in the compound of formula (Ia) as set forth above:

R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R¹²'s, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

Another embodiment is the use where, in the compound of formula (Ia) as set forth above:

R¹, R⁴ and R⁵ are each hydrogen;
R² is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; each R⁹ is independently selected from the group consisting of a direct bond and an optionally substituted straight or branched alkylene chain;
each R¹⁰ is an optionally substituted straight or branched alkylene chain;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In some embodiments in the compound of formula (Ia) as set forth above:

R² is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and
R³ is heteroaryl selected from the group consisting of pyridinyl, pyrimidinyl, 4,5-dihydro-1H-benzo[b]azepin-2(3H)-on-8-yl, benzo[d]imidazolyl, 6,7,8,9-tetrahydro-5H-pyrido[3,2-d]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, thieno[3,2-d]pyrimidinyl and 6,7,8,9-tetrahydro-5H-cyclohepta[b]pyridin-3-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments the compound of formula (Ia), as set forth above, is selected from the group consisting of:
1-(6,7-dimethoxy-quinazolin-4-yl)-N³-(5,7,8,9-tetrahydrospiro[cyclohepta[b]pyridine-6,2′-[1,3]dioxolane]-3-yl)-1H-1,2,4-triazole-3,5-diamine;
1-(2-chloro-7-methylthieno[3,2-d]pyrimidin-4-yl)-N³-(5,7,8,9-tetrahydrospiro[cyclohepta[b]pyridine-6,2′-[1,3]dioxolane]-3-yl)-1H-1,2,4-triazole-3,5-diamine;
1-(2-chloro-7-methylthieno[3,2-d]pyrimidin-4-yl)-N³-(5,6,8,9-tetrahydrospiro[cyclohepta[b]pyridine-7,2′-[1,3]dioxolane]-3-yl)-1H-1,2,4-triazole-3,5-diamine; and
1-(2-chloro-7-methylthieno[3,2-d]pyrimidin-4-yl)-N-(5′,5′-dimethyl-6,8,9,10-9tetrahydro-5H-spiro[cycloocta[b]pyridine-7,2′-[1,3]dioxane]-3-yl)-1H-1,2,4-triazole-3,5-diamine.

In some embodiments in the compound of formula (Ia) as set forth above, R² is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and R¹, R⁴, R$, each R⁶, each R⁷, each R⁸, each R⁹, each R¹⁰, each R¹¹, each R¹², each R¹³ and each R¹⁴ are as described above for compounds of formula (I).

In some embodiments in the compound of formula (Ia) as set forth above:

R¹, R⁴ and R⁵ are each independently hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or two R^{12's}, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In some embodiments in the compound of formula (Ia) as set forth above:

R² is aryl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments in the compound of formula (Ia) as set forth above:

R² is aryl selected from the group consisting of phenyl and 6,7,8,9-tetrahydro-5H-benzo[7]annulene-2-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ia) as set forth above:

R² is phenyl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments in the compound of formula (Ia) as set forth above:

R² is phenyl optionally substituted by one or more substitutents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group consisting of —R⁹—OR⁸, —R⁹—N(R⁶)R⁷, —R⁹—C(O)OR⁶, —R⁹—C(O)N(R⁶)R⁷, —R⁹—N(R⁶)C(O)R⁷, —R⁹—N(R⁶)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
R³ is selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7-dihydro-5H-benzo[6,7]cyclohepta[1,2-b]pyridin-2-yl, 6,7-dihydro-5H-benzo[2,3]thiepino[4,5-c]pyridazin-3-yl, spiro[chromeno[4,3-c]pyridazine-5,1′-cyclopentane]-3-yl and 6,7-dihydro-5H-benzo[6,7]cyclohepta[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
N³-(4-(4-cyclohexanylpiperazin-1-yl)phenyl)-1-(6,7-dihydro-5H-benzo[6,7]cyclohepta[1,2-c]pyridazin-3-yl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(pyrrolidin-1-yl)piperidin-1-yl)-1H-1,2,4-triazole-3,5-diamine;
1-(6,7-dihydro-5H-benzo[6,7]cyclohepta[1,2-c]pyridazin-3-yl)-N³-(3-fluoro-4-(4-methyl-3-phenylpiperazin-1-yl)phenyl)-1H-1,2,4-triazole-3,5-diamine;
1-(6,7-dihydro-5H-benzo[6,7]cyclohepta[1,2-c]pyridazin-3-yl)-N-(3-fluoro-(4-(4-piperidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(indolin-2-on-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(morpholin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-cyclopentyl-2-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(3,5-dimethylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-(pyrrolidin-1-yl)piperidin-1-yl)-3-cyanophenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(diethylamino)pyrrolidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-(bicyclo[2.2.1]heptan-2-yl)piperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(4-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(diethylamino)piperidin-1-yl)phenyl)-1H-1,2,4-triazole-3,5-diamine;
1-(6,7-dihydro-5H-9-methoxybenzo[6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(4-(cyclohexyl)piperazin-1-yl)phenyl)-1H-1,2,4-triazole-3,5-diamine;
1-(6,7-dihydro-5H-9-methoxybenzo[6,7]cyclohepta[1,2-c]pyridazin-3-yl)-N³-(3-fluoro-4-(4-(cyclohexyl)piperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-dimethylaminopiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-chloro-4-(4-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-trifluoromethyl-4-(4-pyrrolidin-1-yl-piperidin-1-yl)phenyl)-1H-1,2,4-triazole-3,5-diamine;
1-(6,7-dihydro-5H-9,10-dimethoxybenzo[6,7]cyclohepta[1,2-c]pyridazin-3-yl)-N³-(3-fluoro-4-(4-pyrrolidin-1-yl-piperidin-1-yl)phenyl)-1H-1,2,4-triazole-3,5-diamine;
1-(6,7-dihydro-5H-9,10, 11-trimethoxybenzo[6,7]cyclohepta[1,2-c]pyridazin-3-yl)-N³-(3-fluoro-4-(4-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N-(3-fluoro-4-(5-methyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-yl-piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-yl-azepan-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-N-methylpiperidin-4-yl-piperidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-(pyrrolidinyl)piperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(5-propyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(decahydropyrazino[1,2-a]azepin-2-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(5-cyclopentyloctahydropyrrolo[3,4-c]pyrrolyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(pyrrolidin-1-yl)-8-azabicyclo[3.2.1]oct-8-yl)phenyl)-1H-1,2,4-triazole-3,5-diamine;
1-(6,7-dihydro-5H-benzo[6,7]cyclohepta[1,2-c]pyridazin-3-yl)-N-(3-fluoro-4-(4-pyrrolidin-1-yl-azepan-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-isopropylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(1-methyloctahydropyrrolo[3,4-b]pyrrol-5-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(N-methylcyclopentylamino)piperidinyl)phenyl)-1H-1,2,4-triazole-3,5-diamine;
1-(6,7-dihydro-5H-benzo[6,7]cyclohepta[1,2-c]pyridazin-3-yl)-N-(3-fluoro-4-(4-(dipropylamino)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(1-propyloctahydro-1H-pyrrolo[3,2-c]pyridine-5-yl)phenyl)-1H-1,2,4-triazole-3,5-diamine;
1-(6,7-dihydro-5H-benzo[6,7]cyclohepta[4,5-c]pyridazin-2-yl)-N-(3-fluoro-4-(4-(N-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(tert-butyloxycarbonylamino)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-aminopiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(5-cyclohexyloctahydropyrrolo[3,4-c]pyrrolyl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(methylpiperidin-4-yl)phenyl)-1H-1,2,4-triazole-3,5-diamine;
1-(6,7-dihydro-5H-benzo[6,7]cyclohepta[1,2-c]pyridazin-3-yl)-N³-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-methyl-4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-cyclopentylpiperazinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-N-methylpiperidin-4-ylpiperazinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(7-methyl-2,7-diazaspiro[4.4]nonan-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(N-isopropylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-pyrrolidin-1-ylazetidinyl)phenyl)-1H-1,2,4-triazole-3,5-diamine;
1-(6,7-dihydro-5H-benzo[6,7]cyclohepta[1,2-c]pyridazin-3-yl)-N³-(3-methyl-4-(4-(N-methylpiperazin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-((S)-3-(pyrrolidin-1-ylmethyl)pyrrolidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(pyrrolidinylmethyl)piperidinyl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-((4aR,8aS)-decahydroisoquinolin-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(octahydro-1H-pyrido[1,2-a]pyrazin-2-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-(4-(3-pyrrolidin-1-yl)pyrrolidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(5-methyloctahydropyrrolo[3,4-c]pyrrolyl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(octahydropyrrolo[3,4-c]pyrrolyl)phenyl)-1H-1,2,4-triazole-3,5-diamine;
1-(6,7-dihydro-9-chloro-5H-benzo[6,7]cyclohepta[1,2-c]pyridazin-3-yl)-N³-(3-fluoro-4-(4-pyrrolidin-1-ylpiperidin-1-yl)phenyl)-1H-1,2,4-triazole-3,5-diamine;
1-(6,7-dihydro-9-chloro-5H-benzo[6,7]cyclohepta[1,2-c]pyridazin-3-yl)-N³-(3-fluoro-4-(4-(N-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(4-iodophenyl)-1H-1,2,4-triazole-3,5-diamine;
1-(spiro[chromeno[4,3-c]pyridazine-5,1′-cyclopentane]-3-yl)-N³-(3-fluoro-4-(4-(4-methylpiperazin-1-yl)piperidin-1-yl)phenyl)-1H-1,2,4-triazole-3,5-diamine;
1-(spiro[chromeno[4,3-c]pyridazine-5,1′-cyclopentane]-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-pyrrolidin-1-ylpiperidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-pyrrolidin-1-ylpiperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-(4-methylpiperazin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(3-(3R)-dimethylaminopyrrolidin-1-yl)phenyl)-1H-1,2,4-triazole-3,5-diamine;
1-(6,7-dihydro-5H-benzo[6,7]cyclohepta[4,5-c]pyridazin-2-yl)-N-(3-methyl-4-(4-pyrrolidin-1-ylpiperidin-1-yl)phenyl)-1H-1,2,4-triazole-3,5-diamine;
1-(6,7-dihydro-5H-benzo[6,7]cyclohepta[4,5-c]pyridazin-2-yl)-N³-(3-fluoro-4-(4-pyrrolidin-1-ylpiperidin-1-yl)phenyl)-1H-1,2,4-triazole-3,5-diamine;
1-(4-phenyl-6,7-dihydro-5H-benzo[6,7]cyclohepta[1,2-b]pyridin-2-yl)-N³-(3-fluoro-4-(4-cyclohexylpiperazin-1-yl)phenyl)-1H-1,2,4-triazole-3,5-diamine;
1-(4-phenyl-6,7-dihydro-5H-benzo[6,7]cyclohepta[1,2-b]pyridin-2-yl)-N³-(4-(4-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N³-(4-(1-bicyclo[2.2.1]heptan-2-yl)-piperidin-4-ylphenyl)-1H-1,2,4-triazole-3,5-diamine;
1-(6,7-dihydro-5H-benzo[6,7]cyclohepta[1,2-c]pyridazin-3-yl)-N³-(4-(1-cyclopropylmethylpiperidin-4-yl)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(4-cyclopropylmethylpiperazin-1-yl)phenyl)-1H-1,2,4-triazole-3,5-diamine;
1-(6,7-dihydro-5H-benzo[2,3]thiepino[4,5-c]pyridazin-3-yl)-N³-(4-(1-bicyclo[2.2.1]heptan-2-yl)-piperidin-4-ylphenyl)-1H-1,2,4-triazole-3,5-diamine;
1-(4-phenyl-6,7-dihydro-5H-benzo[6,7]cyclohepta[1,2-b]pyridin-2-yl)-N³-(3-fluoro-4-(4-pyrrolidin-1-ylpiperidin-1-yl)phenyl)-1H-1,2,4-triazole-3,5-diamine, and
1-(6,7-dihydro-5H-pyrido[2′,3′:6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine.

In some embodiments in the compound of formula (Ia) as set forth above:

R² is phenyl optionally substituted by one or more substitutents selected from the group consisting of halo, alkyl, heterocyclylalkenyl, —R¹³—OR¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)N(R¹²)₂, and —R¹³—N(R¹²)C(O)R¹²;
R³ is selected from the group consisting of 6,7-dihydro-5H-benzo[6,7]cyclohepta[1,2-c]pyridazin-3-yl and 6,7-dihydro-5H-benzo[6,7]cyclohepta[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(6,7-dihydro-5H-benzo[6,7]cyclohepta[1,2-c]pyridazin-3-yl)-N³-(4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(4-(4-(cyclopentyl)piperazin-1-ylcarbonyl)phenyl)-1H-1,2,4-triazole-3,5-diamine;
1-(6,7-dihydro-5H-benzo[6,7]cyclohepta[1,2-c]pyridazin-3-yl)-N³-(4-((2-pyrrolidin-1-ylethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-(2,2,6,6-tetramethylpiperidin-1-yl)ethoxyphenyl)-1H-1,2,4-triazole-3,5-diamine;
1-(6,7-dihydro-5H-benzo[6,7]cyclohepta[1,2-c]pyridazin-3-yl)-N³-(4-((2-(dimethylamino)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-((2-(methoxy)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-((2-(pyrrolidin-1-yl)ethyl)aminocarbonyl)phenyl)-1H-1,2,4-triazole-3,5-diamine;
1-(6,7-dihydro-5H-benzo[6,7]cyclohepta[1,2-c]pyridazin-3-yl)-N³-(4-((4-(pyrrolidin-1-yl)piperidin-1-yl)carbonyl)phenyl)-1H-1,2,4-triazole-3,5-diamine;
1-(6,7-dihydro-5H-benzo[6,7]cyclohepta[1,2-c]pyridazin-3-yl)-N³-(3-chloro-4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(2-(pyrrolidin-1-yl)ethoxy)phenyl)-1H-1,2,4-triazole-3,5-diamine;
1-(6,7-dihydro-5H-10-fluorobenzo[6,7]cyclohepta[1,2-c]pyridazin-3-yl)-N³-(3-fluoro-4-(2-(pyrrolidin-1-yl)ethoxy)phenyl)-1H-1,2,4-triazole-3,5-diamine;
1-(6,7-dihydro-5H-9-methoxybenzo[6,7]cyclohepta[1,2-c]pyridazin-3-yl)-N³-(3-fluoro-4-(2-(pyrrolidin-1-yl)ethoxy)phenyl)-1H-1,2,4-triazole-3,5-diamine;
1-(6,7-dihydro-5H-benzo[6,7]cyclohepta[1,2-c]pyridazin-3-yl)-N³-(4-(2-(N-methylcyclopentylamino)ethoxy)phenyl)-1H-1,2,4-triazole-3,5-diamine;
1-(6,7-dihydro-5H-benzo[6,7]cyclohepta[1,2-c]pyridazin-3-yl)-N³-(3-fluoro-4-(N-methylpiperidin-4-yl-N-methylamino)phenyl)-1H-1,2,4-triazole-3,5-diamine;
1-(6,7-dihydro-5H-benzo[6,7]cyclohepta[1,2-c]pyridazin-3-yl)-N³-(4-((N-butyl-N-acetoamino)methyl)phenyl)-1H-1,2,4-triazole-3,5-diamine;
1-(6,7-dihydro-5H-benzo[6,7]cyclohepta[1,2-c]pyridazin-3-yl)-N³-(4-(4-(4-methylpiperazin-1-yl)piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-(piperidin-1-yl)piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(piperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(pyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(3-dimethylaminopyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(3-diethylaminopyrrolidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-pyrrolidin-1-ylpiperidin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-methylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-isopropylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(4-cyclopentylpiperazin-1-ylprop-1-enyl)phenyl)-1H-1,2,4-triazole-3,5-diamine;
1-(6,7-dihydro-5H-benzo[6,7]cyclohepta[1,2-c]pyridazin-3-yl)-N³-(4-(morpholin-4-ylprop-1-enyl)phenyl)-1H-1,2,4-triazole-3,5-diamine; and
1-(6,7-dihydro-5H-benzo[6,7]cyclohepta[1,2-c]pyridazin-3-yl)-N³-(4-(1-methylpiperidin-3-yl-oxy)phenyl)-1H-1,2,4-triazole-3,5-diamine.

In some embodiments in the compound of formula (Ia) as set forth above:

R² is phenyl optionally substituted by one or more substitutents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group consisting of —R⁹—OR⁸, —R⁹—N(R⁶)R⁷, —R⁹—C(O)OR⁶, —R⁹—C(O)N(R⁶)R⁷, —R⁹—N(R⁶)C(O)R⁷, —R⁹—N(R⁶)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; and
R³ is selected from the group consisting of 6,7-dihydro-5H-benzo[6,7]cyclohepta[1,2-d]pyrimidin-4-yl and 6,7-dihydro-5H-benzo[6,7]cyclohepta[1,2-d]pyrimidin-2-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(6,7-dihydro-5H-benzo[6,7]cyclohepta[1,2-d]pyrimidin-4-yl)-N-(4-(4-(bicyclo[2.2.1]heptan-2-yl)piperazin-1-yl)phenyl)-1H-1,2,4-triazole-3,5-diamine;
1-(6,7-dihydro-5H-benzo[6,7]cyclohepta[1,2-d]pyrimidin-4-yl)-N³-(3-fluoro-4-(4-(diethylamino)piperidin-1-yl)phenyl)-1H-1,2,4-triazole-3,5-diamine;
1-(6,7-dihydro-5H-benzo[6,7]cyclohepta[1,2-d]pyrimidin-2-yl)-N-(4-(N-methylpiperazin-1-yl)phenyl)-1H-1,2,4-triazole-3,5-diamine;
1-(6,7-dihydro-5H-benzo[6,7]cyclohepta[1,2-d]pyrimidin-2-yl)-N³-(3-fluoro-4-(4-cyclohexylpiperazinyl)phenyl)-1H-1,2,4-triazole-3,5-diamine; and
1-(6,7-dihydro-5H-benzo[6,7]cyclohepta[1,2-d]pyrimidin-2-yl)-N³-(4-(4-(2S)-bicyclo[2.2.1]heptan-2-yl)-piperazinylphenyl)-1H-1,2,4-triazole-3,5-diamine.

In some embodiments in the compound of formula (Ia) as set forth above:

R² is phenyl optionally substituted by one or more substitutents selected from the group consisting of halo, alkyl, heterocyclylalkenyl, —R¹³—OR¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)N(R¹²)₂, and —R¹³—N(R¹²)C(O)R¹²; and
R³ is selected from the group consisting of 6,7-dihydro-5H-benzo[6,7]cyclohepta[1,2-d]pyrimidin-4-yl and 6,7-dihydro-5H-benzo[6,7]cyclohepta[1,2-d]pyrimidin-2-yl, each optionally substituted by one or more substituents selected from the group consisting of alkyl, aryl, halo and —R⁹—OR^{B}.

In some embodiments the compound of formula (Ia), as set forth above, is selected from the group consisting of:
1-(6,7-dihydro-5H-benzo[6,7]cyclohepta[1,2-d]pyrimidin-2-yl)-N3-(3-fluoro-4-(2-(pyrrolidin-1-yl)ethoxy)phenyl)-1H-1,2,4-triazole-3,5-diamine; and
1-(6,7-dihydro-5H-benzo[6,7]cyclohepta[1,2-d]pyrimidin-4-yl)-N³-(4-(2-(pyrrolidin-1-yl)ethoxy)phenyl)-1H-1,2,4-triazole-3,5-diamine.

In some embodiments in the compound of formula (Ia) as set forth above:

R² is phenyl optionally substituted by one or more substitutents selected from the group consisting of alkyl, halo, haloalkyl, cyano, and optionally substituted heterocyclyl where the optionally substituted heterocyclyl is selected from the group consisting of piperidinyl, piperazinyl, pyrrolidinyl, azepanyl, decahydropyrazino[1,2-a]azepinyl, octahydropyrrolo[3,4-c]pyrrolyl, azabicyclo[3.2.1]octyl, octahydropyrrolo[3,4-b]pyrrolyl, octahydropyrrolo[3,2-c]pyridinyl, 2,7-diazaspiro[4.4]nonanyl and azetidinyl; each independently optionally substituted by one or two substituents selected from the group consisting of —R⁹—OR⁸, —R⁹—N(R⁶)R⁷, —R⁹—C(O)OR⁶, —R⁹—C(O)N(R⁶)R⁷, —R⁹—N(R⁶)C(O)R⁷, —R⁹—N(R⁶)C(O)OR⁷, alkyl, halo, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; and
R³ is selected from the group consisting of 6,7-dihydro-5H-benzo[2,3]azepino[4,5-c]pyridazin-3-yl, (Z)-dibenzo[b,f][1,4]thiazepin-11-yl, 6,7-dihydro-5H-benzo[2,3]oxepino[4,5-c]pyridazin-3-yl, and 6,7-dihydro-5H-benzo[2,3]thiepino[4,5-c]pyridazin-3-yl, each optionally substituted by one or more substituents selected from the group consisting of alkyl, aryl, halo and —R⁹—OR⁸.

In some embodiments the compound of formula (Ia), as set forth above, is selected from the group consisting of:
1-(7-methyl-6,7-dihydro-5H-benzo[2,3]azepino[4,5-c]pyridazin-3-yl)-N-(4-(N-methylpiperazin-1-yl)phenyl)-1H-1,2,4-triazole-3,5-diamine;
1-(7-methyl-6,7-dihydro-5H-benzo[2,3]azepino[4,5-c]pyridazin-3-yl)-N³-(3-fluoro-4-(4-cyclohexylpiperazinyl)phenyl)-1H-1,2,4-triazole-3,5-diamine;
1-((Z)-dibenzo[b,f][1,4]thiazepin-11-yl)-N³-(4-(4-N-methylpiperazinyl)phenyl)-1H-1,2,4-triazole-3,5-diamine;
1-((Z)-dibenzo[b,f][1,4]thiazepin-11-yl)-N³-(3-fluoro-4-(4-diethylaminopiperidin-1-yl)phenyl)-1H-1,2,4-triazole-3,5-diamine;
1-(6,7-dihydro-5H-benzo[2,3]oxepino[4,5-c]pyridazin-3-yl)-N³-(4-(4-pyrrolidin-1-ylpiperidinyl)phenyl)-1H-1,2,4-triazole-3,5-diamine;
1-(6,7-dihydro-5H-benzo[2,3]oxepino[4,5-c]pyridazin-3-yl)-N³-(3-fluoro-4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N³-(3-fluoro-4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N³-(4-(4-pyrrolidin-1-ylpiperidinyl)phenyl)-1H-1,2,4-triazole-3,5-diamine;
1-(6,7-dihydro-5H-benzo[2,3]thiepino[4,5-c]pyridazin-3-yl)-N³-(3-fluoro-4-(4-(pyrrolidinylmethyl)piperidinyl)phenyl)-1H-1,2,4-triazole-3,5-diamine;
1-(6,7-dihydro-5H-benzo[2,3]thiepino[4,5-c]pyridazin-3-yl)-N³-(3-fluoro-4-((4aR,8aS)-decahydroisoquinolin-2-yl)phenyl)-1H-1,2,4-triazole-3,5-diamine; and
1-(6,7-dihydro-5H-benzo[2,3]thiepino[4,5-c]pyridazin-3-yl)-N³-(3-fluoro-4-(octahydro-1H-pyrido[1,2-a]pyrazin-2-yl)phenyl)-1H-1,2,4-triazole-3,5-diamine.

In some embodiments in the compound of formula (Ia) as set forth above:

R² is phenyl optionally substituted by one or more substitutents selected from the group consisting of halo, alkyl, heterocyclylalkenyl, —R¹³—OR¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)N(R¹²)₂, and —R¹³—N(R¹²)C(O)R¹²; and
R³ is selected from the group consisting of 6,7-dihydro-5H-benzo[2,3]azepino[4,5-c]pyridazin-3-yl, (Z)-dibenzo[b,f][1,4]thiazepin-11-yl, 6,7-dihydro-5H-benzo[2,3]oxepino[4,5-c]pyridazin-3-yl, and 6,7-dihydro-5H-benzo[2,3]thiepino[4,5-c]pyridazin-3-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ia), as set forth above, is selected from the group consisting of:
1-(7-methyl-6,7-dihydro-5H-benzo[2,3]azepino[4,5-c]pyridazin-3-yl)-N³-(3-fluoro-4-(2-(pyrrolidin-1-yl)ethoxy)phenyl)-1H-1,2,4-triazole-3,5-diamine; and
1-((Z)-dibenzo[b,f][1,4]thiazepin-11-yl)-N³-(4-(2-(pyrrolidin-1-yl)ethoxy)phenyl)-1H-1,2,4-triazole-3,5-diamine.

In some embodiments in the compound of formula (Ia) as set forth above:

R² is phenyl optionally substituted by a substitutent selected from the group consisting of optionally substituted heterocyclylalkyl, optionally substituted heteroaryl and optionally substituted heteroarylalkyl;
R³ is selected from the group consisting of 6,7-dihydro-5H-benzo[6,7]cyclohepta[1,2-c]pyridazin-3-yl and 6,7-dihydro-5H-benzo[2,3]thiepino[4,5-c]pyridazin-3-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R³, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR¹ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2) each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain; and
R¹² is independently selected from the group consisting of hydrogen, alkyl, haloalkyl, alkenyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl.

In some embodiments the compound of formula (Ia), as set forth above, is selected from the group consisting of:
1-(6,7-dihydro-5H-benzo[6,7]cyclohepta[1,2-c]pyridazin-3-yl)-N³-(4-((4-methylpiperazin-1-yl)methyl)phenyl)-1H-1,2,4-triazole-3,5-diamine;
1-(6,7-dihydro-5H-benzo[6,7]cyclohepta[1,2-c]pyridazin-3-yl)-N³-(4-((5-fluoroindolin-2-on-3-yl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-pyrrolidin-1-ylpiperidinyl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-cyclopentylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N³-(4-((4-isopropylpiperazinyl)methyl)phenyl)-1H-1,2,4-triazole-3,5-diamine; and
1-(6,7-dihydro-5H-benzo[2,3]thiepino[4,5-c]pyridazin-3-yl)-N³-(3-fluoro-4-(isoindolin-2-yl)phenyl)-1H-1,2,4-triazole-3,5-diamine.

In some embodiments in the compound of formula (Ia) as set forth above:

R¹, R⁴ and R⁵ are each independently hydrogen;
R² is 6,7,8,9-tetrahydro-5H-benzo[7]annulene-2-yl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
and each R⁶, each R⁷, each R⁸, each R⁹, each R¹², each R¹⁸ and each R¹⁴ are as described above for compounds of formula (Ia).

In some embodiments the compound of formula (Ia), as set forth above, is selected from the group consisting of:
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((bicyclo[2.2.1]heptan-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((bicyclo[2.2.1]heptan-2-yl)(methyl)amino) 6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-azetidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-diethylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclopentylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(2-(S)-methyloxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(2-(S)-carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(8-diethylaminoethyl-9hydroxy-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(S)-fluoropyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(S)-methylpyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(R)-hydroxypyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(R)-methylpyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(S)-hydroxypyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(3-(R)-fluoropyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-oxo-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclohexylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclopropylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-hydroxy-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-methylpiperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(tetrahydrofuran-2-ylmethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-cyclobutylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(cyclopropylmethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(diethylamino)ethyl)methylamino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-pyrrolidin-1-ylpiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(piperidin-1-ylmethyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(2-(dimethylamino)ethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N-(7-(carboxymethyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N³-((7S)-7-(2-butylamino)-6,7,8,9-tetrahydro-5H-benzo[7]annulene-2-yl)-1H-1,2,4-triazole-3,5-diamine.

In some embodiments in the compound of formula (Ia) as set forth above:

R¹, R⁴ and R⁵ are each independently hydrogen;
R² is heteroaryl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and each R⁶, each R⁷, each R⁸, each R⁹, each R¹², each R¹³ and each R¹⁴ are as described above for compounds of formula (Ia); and each R⁶, each R⁷, each R⁸, each R⁹, each R¹⁰, each R¹¹, each R¹², each R¹³ and each R¹⁴ are as described above for compounds of formula (Ia).

In some embodiments in the compound of formula (Ia) as set forth above:

R² is heteroaryl selected from the group consisting of pyridinyl, pyrimidinyl, 4,5-dihydro-1H-benzo[b]azepin-2(3H)-on-8-yl, benzo[d]imidazolyl, 6,7,8,9-tetrahydro-5H-pyrido[3,2-d]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, and 6,7,8,9-tetrahydro-5H-cyclohepta[b]pyridin-3-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R³, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

Another embodiment is the use where, in the compound of formula (Ia) as set forth above:

R² is selected from the group consisting of pyridinyl and pyrimidinyl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments the compound of formula (Ia), as set forth above, is selected from the group consisting of:
1-(6,7-dihydro-5H-benzo[6,7]cyclohepta[1,2-c]pyridazin-3-yl)-N³-(6-(4-(bicyclo[2.2.1]heptan-2-yl)piperazin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-cyclopentyl-1,4-diazepan-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-methylpiperazin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(4-methylpiperazin-1-yl)piperidin-1-yl)pyridine-3-yl)-1H-1,2,4-triazole-3,5-diamine;
1-(6,7-dihydro-5H-benzo[6,7]cyclohepta[1,2-c]pyridazin-3-yl)-N³-(pyridin-3-yl)-1H-1,2,4-triazole-3,5-diamine;
1-(6,7-dihydro-5H-benzo[6,7]cyclohepta[1,2-c]pyridazin-3-yl)-N³-(6-(6-aminopyridin-3-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-aminophenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-cyanophenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(benzo[d][1,3]dioxole-6-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(3-methylsulfonamidylphenyl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(2-diethylaminomethyl)pyrrolidin-1-ylpyridin-3-yl)-1H-1,2,4-triazole-3,5-diamine;
1-(6,7-dihydro-5H-benzo[6,7]cyclohepta[1,2-c]pyridazin-3-yl)-N³-(6-(3-diethylaminopyrrolidin-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-(N-methylpiperazin-4-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(pyrrolidin-1-yl)piperidin-1-yl)-5-methylpyridin-3-yl)-1H-1,2,4-triazole-3,5-diamine;
1-(6,7-dihydro-5H-benzo[6,7]cyclohepta[1,2-c]pyridazin-3-yl)-N³-(6-(3-piperidin-1-yl-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(bicyclo[2.2.1]heptan-2-yl)-1,4-diazepan-1-yl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-(pyrrolidin-1-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-piperidin-1-yl)-propanylpyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-(piperidin-1-yl)piperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(6-(3-(4-dimethylaminopiperidin-1-yl)-(E)-propenyl)pyridin-3-yl)-1H-1,2,4-triazole-3,5-diamine;
1-(6,7-dihydro-5H-benzo[6,7]cyclohepta[1,2-c]pyridazin-3-yl)-N³-(2-(4-pyrrolidin-1-ylpiperidin-1-yl)pyrimidin-5-yl)-1H-1,2,4-triazole-3,5-diamine;
1-(6,7-dihydro-5H-benzo[6,7]cyclohepta[1,2-c]pyridazin-3-yl)-N³-(2-(4-(piperidin-1-ylmethyl)piperidin-1-yl)pyrimidin-5-yl)-1H-1,2,4-triazole-3,5-diamine;
1-(6,7-dihydro-5H-benzo[6,7]cyclohepta[1,2-c]pyridazin-3-yl)-N³-(6-((4-piperidin-1-ylpiperidin-1-yl)carbonyl)pyridin-3-yl)-1H-1,2,4-triazole-3,5-diamine;
1-(6,7-dihydro-5H-benzo[6,7]cyclohepta[1,2-c]pyridazin-3-yl)-N-(2-(4-cyclopropylmethylpiperazin-1-yl)pyridine-5-yl)-1H-1,2,4-triazole-3,5-diamine; and
1-(6,7-dihydro-5H-benzo[6,7]cyclohepta[1,2-c]pyridazin-3-yl)-N³-(2-(3-(S)-methyl-4-cyclopropylmethylpiperazin-1-yl)pyridine-5-yl)-1H-1,2,4-triazole-3,5-diamine.

In some embodiments in the compound of formula (Ia) as set forth above:

R¹, R⁴ and R⁵ are each independently hydrogen;
R² is selected from the group consisting of 4,5-dihydro-1H-benzo[b]azepin-2(3H)-on-8-yl, benzo[d]imidazolyl, 6,7,8,9-tetrahydro-5H-pyrido[3,2-d]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, and 6,7,8,9-tetrahydro-5H-cyclohepta[b]pyridin-3-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ia), as set forth above, is selected from the group consisting of:
1-(6,7-dihydro-5H-benzo[6,7]cyclohepta[1,2-c]pyridazin-3-yl)-N³-(4,5-dihydro-1H-benzo[b]azepin-2(3H)-on-8-yl)-1H-1,2,4-triazole-3,5-diamine;
1-(6,7-dihydro-5H-benzo[6,7]cyclohepta[1,2-c]pyridazin-3-yl)-N³-(2-(dimethylaminomethyl)-1H-benzo[d]imidazol-5-yl)-1H-1,2,4-triazole-3,5-diamine;
1-(6,7-dihydro-5H-benzo[6,7]cyclohepta[1,2-c]pyridazin-3-yl)-N³-(7-cyclopentyl-6,7,8,9-tetrahydro-5H-pyrido[3,2-d]azepin-3-yl)-1H-1,2,4-triazole-3,5-diamine;
1-(6,7-dihydro-5H-benzo[6,7]cyclohepta[1,2-c]pyridazin-3-yl)-N³-(6-methyl-5,6,7,8-tetrahydro-1,6-naphthyridin-3-yl)-1H-1,2,4-triazole-3,5-diamine;
1-(6,7-dihydro-5H-benzo[6,7]cyclohepta[1,2-c]pyridazin-3-yl)-N³-(6-(4-(4-methylpiperazin-1-yl)piperidin-1-yl)pyridine-3-yl)-1H-1,2,4-triazole-3,5-diamine;
1-(6,7-dihydro-5H-benzo[6,7]cyclohepta[1,2-c]pyridazin-3-yl)-N³-(6-(4-methylpiperazin-1-yl)carbonyl-5,6,7,8-tetrahydroquinolin-3-yl)-1H-1,2,4-triazole-3,5-diamine, compound #31, 1H-1,2,4-triazole-3,5-diamine;
1-(6,7-dihydro-5H-benzo[6,7]cyclohepta[1,2-c]pyridazin-3-yl)-N³-(2,3,4,5-tetrahydrobenzo[b]oxepin-7-yl)-1H-1,2,4-triazole-3,5-diamine;
1-(6,7-dihydro-5H-benzo[6,7]cyclohepta[1,2-c]pyridazin-3-yl)-N³-(3,4-dihydro-2H-benzo[b][1,4]dioxepin-7-yl)-1H-1,2,4-triazole-3,5-diamine;
1-(6,7-dihydro-5H-benzo[2,3]oxepino[4,5-c]pyridazin-3-yl)-N³-(2-(pyrrolidin-1-ylmethyl)benzo[d]oxazol-5-yl)-1H-1,2,4-triazole-3,5-diamine;
1-(6,7-dihydro-5H-benzo[6,7]cyclohepta[1,2-c]pyridazin-3-yl)-N³-(4-(2-dimethylaminoethyl)-(3,4-dihydro-2H-benzo[b][1,4]oxazin-7-yl))-1H-1,2,4-triazole-3,5-diamine;
1-(6,7-dihydro-5H-benzo[2,3]oxepino[4,5-c]pyridazin-3-yl)-N³-(4-(2-dimethylaminoethyl)-(3,4-dihydro-2H-benzo[b][1,4]oxazin-7-yl))-1H-1,2,4-triazole-3,5-diamine;
1-(6,7-dihydro-5H-benzo[2,3]thiepino[4,5-c]pyridazin-3-yl)-N³-(2-(1-(4-(2-(dimethylamino)ethyl)piperazin-1-yl)oxomethyl)benzo[b]thiophen-5-yl)-1H-1,2,4-triazole-3,5-diamine;
1-(6,7-dihydro-5H-benzo[6,7]cyclohepta[1,2-c]pyridazin-3-yl)-N³-(6-cyclopentyl-6,7,8,9-tetrahydro-5H-pyrido[3,2-c]azepin-3-yl)-1H-1,2,4-triazole-3,5-diamine;
1-(6,7-dihydro-5H-benzo[6,7]cyclohepta[1,2-c]pyridazin-3-yl)-N³-((7-pyrrolidin-1-yl)-6,7,8,9-tetrahydro-5H-cyclohepta[b]pyridine-3-yl)-1H-1,2,4-triazole-3,5-diamine;
1-(6,7-dihydro-5H-benzo[6,7]cyclohepta[1,2-c]pyridazin-3-yl)-N³ (2-cyclopentyl-1,2,3,4-tetrahydroisoquinolin-7-yl)-1H-1,2,4-triazole-3,5-diamine;
1-(6,7-dihydro-5H-benzo[6,7]cyclohepta[1,2-c]pyridazin-3-yl)-N³-(6-(pyrrolidin-1-yl)-5,6,7,8-tetrahydroquinolin-3-yl)-1H-1,2,4-triazole-3,5-diamine;
1-(6,7-dihydro-5H-benzo[6,7]cyclohepta[1,2-c]pyridazin-3-yl)-N-(6-cyclopentyl-5,6,7,8-tetrahydro-1,6-naphthyridine-3-yl)-1H-1,2,4-triazole-3,5-diamine;
1-(6,7-dihydro-5H-benzo[6,7]cyclohepta[1,2-c]pyridazin-3-yl)-N³-((S)-7-(pyrrolidin-1-yl)-6,7,8,9-tetrahydro-5H-cyclohepta[b]pyridine-3-yl)-1H-1,2,4-triazole-3,5-diamine;
1-(6,7-dihydro-5H-benzo[6,7]cyclohepta[1,2-c]pyridazin-3-yl)-N³-(1,2,3,4-tetrahydroisoquinolin-7-yl)-1H-1,2,4-triazole-3,5-diamine;
1-(6,7-dihydro-5H-benzo[6,7]cyclohepta[1,2-c]pyridazin-3-yl)-N³-(2-(1-methylpiperidin-4-yl)-1,2,3,4-tetrahydroisoquinolin-7-yl)-1H-1,2,4-triazole-3,5-diamine; and
1-(6,7-dihydro-5H-benzo[6,7]cyclohepta[1,2-c]pyridazin-3-yl)-NL-(2-(cyclopropylmethyl)-1,2,3,4-tetrahydroisoquinolin-7-yl)-1H-1,2,4-triazole-3,5-diamine.

In some embodiments the compound of formula (Ia), as set forth above, is a compound of formula (Ia1):

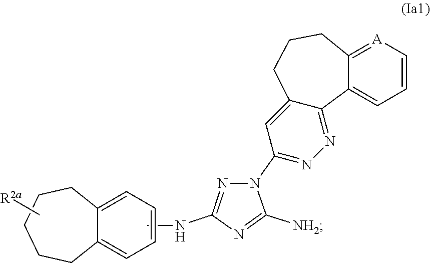

wherein:

### A is ═C(H)— or ═N—;

each R^{2a} is independently selected from the group consisting of —N(R^{12a})₂ and —N(R^{12a})C(O)R^{12a},
or R^{2a} is an N-heterocyclyl optionally substituted by one or more substituents selected from the group consisting of halo and —R²¹—C(O)OR²⁰,
each R^{12a} is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl;
R²⁰ is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl; and
R²¹ is independently selected from the group consisting of a direct bond or an optionally substituted straight or branched alkylene chain;
as an isolated stereoisomer or mixture thereof, or a pharmaceutically acceptable salt thereof.

In some embodiments the compound of formula (I) is a compound of formula (Ib):

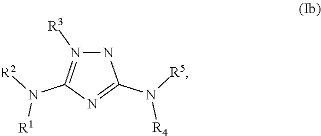

wherein R¹, R², R³, R⁴ and R⁵ are as described above for compounds of formula (I), as an isolated stereoisomer or mixture thereof or as a tautomer or mixture thereof, or a pharmaceutically acceptable salt or N-oxide thereof.

In some embodiments in the compound of formula (Ib) as set forth above, R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); and R¹, R⁴, R⁵, each R⁶, each R⁷, each R⁸, each R⁹, each R¹⁰, each R¹¹ and R¹² are as described above in relation to formula (I).

In some embodiments in the compound of formula (Ib) as set forth above:

R¹, R⁴ and R⁵ are each hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl; each R⁹ is independently selected from the group consisting of a direct bond and an optionally substituted straight or branched alkylene chain;
each R¹⁰ is an optionally substituted straight or branched alkylene chain; and
each R¹¹ is independently selected from the group consisting of hydrogen, alkyl, cyano, nitro and —OR⁸.

In some embodiments in the compound of formula (Ib) as set forth above:

R² and R⁸ are each independently a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)₉R⁸ (where p is 0, 1 or 2), and —R⁹—S(O)ₜN(R⁶)R⁷ (where t is 1 or 2).

In some embodiments the compound of formula (Ib), as set forth above, is 1-(6,7-dihydro-5H-benzo[6,7]cyclohepta[1,2-c]pyridazin-3-yl)-N⁵-(5,7,8,9-tetrahydrospiro[cyclohepta[b]pyridine-6,2′[1,3]dioxolane]-3-yl)-1H-1,2,4-triazole-3,5-diamine.

In some embodiments in the compound of formula (Ib) as set forth above:

R² is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁶—S(O)ₚR⁸ (where p is 0, 1 or 2), and —R⁹—S(O)ₜN(R⁶)R⁷ (where t is 1 or 2).

In some embodiments in the compound of formula (Ib) as set forth above:

R¹, R⁴ and R⁵ are each independently hydrogen;
each R⁶ and R⁷ is independently selected from the group consisting of hydrogen, alkyl, alkenyl, alkynyl, haloalkyl, hydroxyalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, and optionally substituted heteroarylalkyl;
each R⁹ is independently selected from the group consisting of a direct bond and an optionally substituted straight or branched alkylene chain;
each R¹⁰ is an optionally substituted straight or branched alkylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R¹²'s, together with the common nitrogen to which they are both attached, form an optionally substituted N-heterocyclyl or an optionally substituted N-heteroaryl;
each R¹³ is independently selected from the group consisting of a direct bond and an optionally substituted straight or branched alkylene chain; and
each R¹⁴ is an optionally substituted straight or branched alkylene chain.

In some embodiments in the compound of formula (Ib) as set forth above:

R² is aryl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:

R¹, R⁴ and R⁵ are each independently hydrogen;
R² is aryl selected from the group consisting of phenyl and 6,7,8,9-tetrahydro-5H-benzo[7]annulene-2-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁰—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:

R² is phenyl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N⁵-(3-fluoro-4-(4-(indolin-2-on-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-(morpholin-4-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(4-(3,5-dimethylpiperazin-1-yl)phenyl)-1H-1,2,4-triazole-3,5-diamine;
1-(7-methyl-6,7-dihydro-5H-benzo[2,3]azepino[4,5-c]pyridazin-3-yl)-N-(4-(N-methylpiperazin-1-yl)phenyl)-1H-1,2,4-triazole-3,5-diamine;
1-(6,7-dihydro-5H-benzo[6,7]cyclohepta[1,2-c]pyridazin-3-yl)-N⁵-(4-((5-fluoroindolin-2-on-3-yl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-pyrrolidin-1-ylpiperidinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-cyclopentylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(4-((4-isopropylpiperazinyl)methyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-N-methylpiperid-4-ylpiperazinyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(7-methyl-2,7-diazaspiro[4.4]nonan-2-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(3-pyrrolidin-1-ylazetidinyl)phenyl)-1H-1,2,4-triazole-3,5-diamine;
1-(6,7-dihydro-5H-benzo[6,7]cyclohepta[1,2-c]pyridazin-3-yl)-N⁵-(3-methyl-4-(4-(N-methylpiperazin-4-yl)piperidin-1-yl)phenyl)-1H-1,2,4-triazole-3,5-diamine;
1-(6,7-dihydro-5H-benzo[2,3]thiepino[4,5-c]pyridazin-3-yl)-N⁵-(4-(4-pyrrolidin-1-ylpiperidinyl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-(4-(3-pyrrolidin-1-yl)pyrrolidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(3-fluoro-4-(4-methylpiperazin-1-yl)phenyl)-1H-1,2,4-triazole-3,5-diamine; and
1-(6,7-dihydro-5H-benzo[6,7]cyclohepta[1,2-c]pyridazin-3-yl)-N⁵-(3-fluoro-4-(4-cyclopropylmethylpiperazin-1-yl)phenyl)-1H-1,2,4-triazole-3,5-diamine.

In some embodiments in the compound of formula (Ib) as set forth above:

R¹, R⁴ and R⁵ are each independently hydrogen;
R² is 6,7,8,9-tetrahydro-5H-benzo[7]annulene-2-yl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N⁵-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N⁵-(7-((bicyclo[2.2.1]heptan-2-yl)(methyl)amino)-6,7,8,9-tetrahydro-5H-benzo[7]annulene-2-yl)-1H-1,2,4-triazole-3,5-diamine; and
1-(6,7-dihydro-5H-benzo[6,7]cyclohepta[1,2-c]pyridazin-3-yl)-N⁵-(7-(S)-pyrrolidin-1-yl-6,7,8,9-tetrahydro-5H-benzo[7]annulene-2-yl)-1H-1,2,4-triazole-3,5-diamine.

In some embodiments in the compound of formula (Ib) as set forth above:

R¹, R⁴ and R⁵ are each independently hydrogen;
R² is heteroaryl optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments in the compound of formula (Ib) as set forth above:

R² is heteroaryl selected from the group consisting of pyridinyl, pyrimidinyl, 4,5-dihydro-1H-benzo[b]azepin-2(3H)-on-8-yl, benzo[d]imidazolyl, 6,7,8,9-tetrahydro-5H-pyrido[3,2-c]azepin-3-yl, 6,7,8,9-tetrahydro-5H-pyrido[3,2-c]azepin-3-yl, 5,6,7,8-tetrahydro-1,6-naphthyridin-3-yl, 5,6,7,8-tetrahydroquinolin-3-yl, 1,2,3,4-tetrahydroisoquinolin-7-yl, 2,3,4,5-tetrahydrobenzo[b]oxepin-7-yl, 3,4-dihydro-2H-benzo[b][1,4]dioxepin-7-yl, benzo[d]oxazol-5-yl, 3,4-dihydro-2H-benzo[b][1,4]oxazin-7-yl, benzo[b]thiophenyl, and 6,7,8,9-tetrahydro-5H-cyclohepta[b]pyridin-3-yl, each optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl, optionally substituted heteroarylalkenyl, —R¹³—OR¹², —R¹³—OC(O)—R¹², —R¹³—O—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); and
R³ is a polycyclic heteroaryl containing more than 14 ring atoms selected from the group consisting of 6,7-dihydro-5H-benzo[6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl, each optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR¹², —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2).

In some embodiments the compound of formula (Ib), as set forth above, is selected from the group consisting of:
1-(6,7-dihydro-5H-benzo[6,7]cyclohepta[1,2-c]pyridazin-3-yl)-N⁵-(6-(4-(pyrrolidin-1-yl)piperidin-1-yl)-5-methylpyridin-3-yl)-1H-1,2,4-triazole-3,5-diamine;
1-(6,7-dihydro-5H-benzo[6,7]cyclohepta[1,2-c]pyridazin-3-yl)-N⁵-(4-(3,5-dimethylpiperazin-1-yl)phenyl)-1H-1,2,4-triazole-3,5-diamine;
1-(6,7-dihydro-5H-benzo[6,7]cyclohepta[1,2-c]pyridazin-3-yl)-N⁵-(1,2,3,4-tetrahydroisoquinolin-7-yl)-1H-1,2,4-triazole-3,5-diamine; and
1-(6,7-dihydro-5H-benzo[6,7]cyclohepta[1,2-c]pyridazin-3-yl)-N⁵-(2-(1-methylpiperidin-4-yl)-1,2,3,4-tetrahydroisoquinolin-7-yl)-1H-1,2,4-triazole-3,5-diamine.

In some embodiments the compound of formula (Ib), as set forth above, is a compound of formula (Ib1):

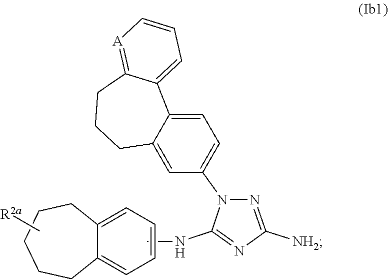

wherein:

### A is ═C(H)— or ═N—;

each R^{2a} is independently selected from the group consisting of —N(R^{12a})₂ and —N(R^{12a})C(O)R^{12a},
or R^{2a} is an N-heterocyclyl optionally substituted by one or more substituents selected from the group consisting of halo and —R²¹—C(O)OR²⁰,
each R^{12a} is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl;
R²⁰ is independently selected from the group consisting of hydrogen, alkyl, alkenyl, optionally substituted aralkyl, optionally substituted cycloalkyl, optionally substituted cycloalkylalkyl, optionally substituted heteroaryl and optionally substituted heteroarylalkyl; and
R²¹ is independently selected from the group consisting of a direct bond or an optionally substituted straight or branched alkylene chain;
as an isolated stereoisomer or mixture thereof, or a pharmaceutically acceptable salt thereof.

### PREFERRED EMBODIMENTS

Preferably, the AXL inhibitor is 1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine.

The most preferred AXL inhibitor is bemcentinib (CAS No. 1037624-75-1; UNII OICW2LX8AS)

### Other Embodiments

In some other embodiments the AXLi is selected from the group consisting of:
Dubermatinib (CAS No. 1341200-45-0; UNII 14D65TV20J);
Gilteritinib (CAS No. 1254053-43-4; UNII 66D92MGC8M);
Cabozantinib (CAS No. 849217-68-1; UNII 1C39JW444G);
SGI7079 (CAS No. 1239875-86-5);
Merestinib (CAS No. 1206799-15-6; UNII 5OGS5K699E);
Amuvatinib (CAS No. 850879-09-3; UNII SO9S6QZB4R);
Bosutinib (CAS No. 380843-75-4; UNII 5018V4AEZ0);

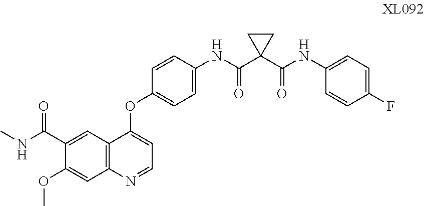 [SMILES: CNC(=O)c1cc2c(Oc3ccc(NC(=O)C4(C(=O)Nc5ccc(F)cc5)CC4)cc3)ccnc2cc1OC]

XL092 from Exelixis
Sitravatinib (CAS No. 1123837-84-2; UNII CWG62Q1VTB);
Glesatinib (CAS No. 936694-12-1; UNII 7Q29OXD98N); and
foretinib (CAS No. 849217-64-7; UNII 81FH7VK1C4).

### Definitions

As used herein, unless specified to the contrary, the following terms have the meaning indicated:

“Amino” refers to the —NH₂ radical.

“Carboxy” refers to the —C(O)OH radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms and which is attached to the rest of the molecule by a single bond, for example, methyl, ethyl, n-propyl, 1-methylethyl (iso-propyl), n-butyl, n-pentyl, 1,1-dimethylethyl (t-butyl), 3-methylhexyl, 2-methylhexyl, and the like. For purposes of this disclosure, the term “lower alkyl” refers to an alkyl radical having one to six carbon atoms.

“Optionally substituted alkyl” refers to an alkyl radical, as defined above, which is optionally substituted by one or more substituents selected from the group consisting of halo, cyano, nitro, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.

“Alkenyl” refers to a straight or branched hydrocarbon chain radical consisting solely of carbon and hydrogen atoms, containing at least one double bond, having from two to twelve carbon atoms, preferably one to eight carbon atoms and which is attached to the rest of the molecule by a single bond, for example, ethenyl, prop-1-enyl, but-1-enyl, pent-1-enyl, and penta-1,4-dienyl.

“Optionally substituted alkenyl” refers to an alkenyl radical, as defined above, which is optionally substituted by one or more substituents selected from the group consisting of halo, cyano, nitro, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.

“Alkynyl” refers to a straight or branched hydrocarbon chain radical consisting solely of carbon and hydrogen atoms, containing at least one triple bond, optionally containing at least one double bond, having from two to twelve carbon atoms, preferably one to eight carbon atoms and which is attached to the rest of the molecule by a single bond, for example, ethynyl, propynyl, butynyl, pentynyl, and hexynyl.

“Optionally substituted alkynyl” refers to an alkynyl radical, as defined above, which is optionally substituted by one or more substituents selected from the group consisting of halo, cyano, nitro, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.

“Straight or branched alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, and n-butylene. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain.

“Optionally substituted straight or branched alkylene chain” refers to an alkylene chain, as defined above, which is optionally substituted by one or more substituents selected from the group consisting of halo, cyano, nitro, aryl, cycloalkyl, heterocyclyl, heteroaryl, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.

“Straight or branched alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, and n-butenylene. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain.

“Optionally substituted straight or branched alkenylene chain” refers to an alkenylene chain, as defined above, which is optionally substituted by one or more substituents selected from the group consisting of halo, cyano, nitro, aryl, cycloalkyl, heterocyclyl, heteroaryl, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.

“Straight or branched alkynylene chain” refers to a straight or branched divalent hydrocarbon chain linking the rest of the molecule to a radical group, consisting solely of carbon and hydrogen, containing at least one triple bond and having from two to twelve carbon atoms, for example, propynylene, and n-butynylene. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain.

“Optionally substituted straight or branched alkynylene chain” refers to an alkynylene chain, as defined above, which is optionally substituted by one or more substituents selected from the group consisting of alkyl, alkenyl, halo, haloalkenyl, cyano, nitro, aryl, cycloalkyl, heterocyclyl, heteroaryl, oxo, thioxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²⁰, —N(R²⁰)C(O)R²⁰, —N(R²⁰)S(O)₂R²⁰, —S(O)ₜOR²⁰ (where t is 1 or 2), —S(O)ₚR²⁰ (where p is 0, 1 or 2), and —S(O)₂N(R²⁰)₂ where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl.

“Aryl” refers to a hydrocarbon ring system radical comprising hydrogen, 6 to 14 carbon atoms and at least one aromatic ring. For purposes of this disclosure, the aryl radical may be a monocyclic, bicyclic, or tricyclic system and which may include spiro ring systems. An aryl radical is commonly, but not necessarily, attached to the parent molecule via an aromatic ring of the aryl radical. For purposes of this disclosure, an “aryl” radical as defined herein can not contain rings having more than 7 members and cannot contain rings wherein two non-adjacent ring atoms thereof are connected through an atom or a group of atoms (i.e., a bridged ring system). Aryl radicals include, but are not limited to, aryl radicals derived from acenaphthylene, anthracene, azulene, benzene, 6,7,8,9-tetrahydro-5H-benzo[7]annulene, fluorene, as-indacene, s-indacene, indane, indene, naphthalene, phenalene, and phenanthrene.

“Optionally substituted aryl” refers to an aryl radical, as defined above, which is optionally substituted by one or more substituents selected from the group consisting of alkyl, alkenyl, alkynyl, halo, haloalkyl, haloalkenyl, haloalkynyl, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—O—R²²—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl, each R²¹ is independently a direct bond or a straight or branched alkylene or alkenylene chain, and R²² is a straight or branched alkylene or alkenylene chain.

“Aralkyl” refers to a radical of the formula —R_{b}—R_{c} where R_{b} is an alkylene chain as defined above and R_{c} is one or more aryl radicals as defined above, for example, benzyl and diphenylmethyl.

“Optionally substituted aralkyl” refers to an aralkyl radical, as defined above, wherein the alkylene chain of the aralkyl radical is an optionally substituted alkylene chain, as defined above, and each aryl radical of the aralkyl radical is an optionally substituted aryl radical, as defined above.

“Aralkenyl” refers to a radical of the formula —R_{d}—R_{c} where R_{d} is an alkenylene chain as defined above and R_{c} is one or more aryl radicals as defined above.

“Optionally substituted aralkenyl” refers to an aralkenyl radical, as defined above, wherein the alkenylene chain of the aralkenyl radical is an optionally substituted alkenylene chain, as defined above, and each aryl radical of the aralkenyl radical is an optionally substituted aryl radical, as defined above.

“Aralkynyl” refers to a radical of the formula —RₑR_{c} where Rₑ is an alkynylene chain as defined above and R_{c} is one or more aryl radicals as defined above.

“Optionally substituted aralkynyl” refers to an aralkynyl radical, as defined above, wherein the alkynylene chain of the aralkynyl radical is an optionally substituted alkynylene chain, as defined above, and each aryl radical of the aralkynyl radical is an optionally substituted aryl radical, as defined above.

“Cycloalkyl” refers to a stable non-aromatic monocyclic or polycyclic hydrocarbon radical consisting solely of carbon and hydrogen atoms, which includes fused, spiro or bridged ring systems, having from three to fifteen carbon atoms, preferably having from three to ten carbon atoms, more preferably from five to seven carbons and which is saturated or unsaturated and attached to the rest of the molecule by a single bond. For purposes of this disclosure, a bridged ring system is a system wherein two non-adjacent ring atoms thereof are connected through an atom or a group of atoms, wherein the atom or the group of atoms are the bridging element. An example of a bridged cycloalkyl (monovalent) radical is norbornanyl (also called bicyclo[2.2.1]heptanyl). For purposes of this disclosure, a non-bridged ring system is a system which does not contain a bridging element, as described above. For purposes of this disclosure, a fused ring system is a system wherein two adjacent ring atoms thereof are connected through an atom or a group of atoms. An example of a fused cycloalkyl (monovalent) radical is decahydronaphthalenyl (also called decalinyl). For purposes of this disclosure, a spiro ring system is a system wherein two rings are joined via a single carbon (quaternary) atom. An example of a spiro cycloalkyl (monovalent) radical is spiro[5.5]undecanyl. Monocyclic cycloalkyl radicals do not include spiro, fused or bridged cycloalkyl radicals, but do include for example, cyclopropyl, cyclobutyl, cyclopentyl, cyclohexyl, cycloheptyl, and cyclooctyl. Polycyclic radicals include fused, spiro or bridged cycloalkyl radicals, for example, C₁₀ radicals such as adamantanyl (bridged) and decalinyl (fused), and C₇ radicals such as bicyclo[3.2.0]heptanyl (fused), norbornanyl and norbornenyl (bridged), as well as substituted polycyclic radicals, for example, substituted C₇ radicals such as 7,7-dimethylbicyclo[2.2.1]heptanyl (bridged).

“Optionally substituted cycloalkyl” refers to a cycloalkyl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl, and each R²¹ is independently a direct bond or a straight or branched alkylene or alkenylene chain.

“Cycloalkylalkyl” refers to a radical of the formula —R_{b}R_{g} where R_{b} is an alkylene chain as defined above and R_{g} is a cycloalkyl radical as defined above.

“Optionally substituted cycloalkylalkyl” refers to a cycloalkylalkyl radical, as defined above, wherein the alkylene chain of the cycloalkylalkyl radical is an optionally substituted alkylene chain, as defined above, and the cycloalkyl radical of the cycloalkylalkyl radical is an optionally substituted cycloalkyl radical, as defined above.

“Cycloalkylalkenyl” refers to a radical of the formula —R_{d}R_{g} where R_{d} is an alkenylene chain as defined above and R_{g} is a cycloalkyl radical as defined above.

“Optionally substituted cycloalkylalkenyl” refers to a cycloalkylalkenyl radical, as defined above, wherein the alkenylene chain of the cycloalkylalkenyl radical is an optionally substituted alkenylene chain, as defined above, and the cycloalkyl radical of the cycloalkylalkenyl radical is an optionally substituted cycloalkyl radical as defined above.

“Cycloalkylalkynyl” refers to a radical of the formula —RₑR_{g} where Rₑ is an alkynylene radical as defined above and R_{g} is a cycloalkyl radical as defined above.

“Optionally substituted cycloalkylalkynyl” refers to a cycloalkylalkynyl radical, as defined above, wherein the alkynylene chain of the cycloalkylalkynyl radical is an optionally substituted alkynylene chain, as defined above, and the cycloalkyl radical of the cycloalkylalkynyl radical is an optionally substituted cycloalkyl radical as defined above.

“Halo” refers to bromo, chloro, fluoro or iodo.

“Haloalkyl” refers to an alkyl radical, as defined above, that is substituted by one or more halo radicals, as defined above, for example, trifluoromethyl, difluoromethyl, trichloromethyl, 2,2,2-trifluoroethyl, 1-fluoromethyl-2-fluoroethyl, 3-bromo-2-fluoropropyl, and 1-bromomethyl-2-bromoethyl.

“Haloalkenyl” refers to an alkenyl radical, as defined above, that is substituted by one or more halo radicals, as defined above.

“Haloalkynyl” refers to an alkynyl radical, as defined above, that is substituted by one or more halo radicals, as defined above.

“Heterocyclyl” refers to a stable 3- to 18-membered non-aromatic ring system radical which comprises one to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include spiro or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of a bridged heterocyclyl include, but are not limited to, azabicyclo[2.2.1]heptanyl, diazabicyclo[2.2. 1]heptanyl, diazabicyclo[2.2.2]octanyl, diazabicyclo[3.2.1]octanyl, diazabicyclo[3.3.1]nonanyl, diazabicyclo[3.2.2]nonanyl and oxazabicyclo[2.2.1]heptanyl. A “bridged N-heterocyclyl” is a bridged heterocyclyl containing at least one nitrogen, but which optionally contains up to four additional heteroatoms selected from O, N and S. For purposes of this disclosure, a non-bridged ring system is a system wherein no two non-adjacent ring atoms thereof are connected through an atom or a group of atoms. Examples of heterocyclyl radicals include, but are not limited to, dioxolanyl, 1,4-diazepanyl, decahydroisoquinolyl, imidazolinyl, imidazolidinyl, isothiazolidinyl, isoxazolidinyl, morpholinyl, octahydroindolyl, octahydroisoindolyl, octahydro-1H-pyrrolo[3,2-c]pyridinyl, octahydro-1H-pyrrolo[2,3-c]pyridinyl, octahydro-1H-pyrrolo[2,3-b]pyridinyl, octahydro-1H-pyrrolo[3,4-b]pyridinyl, octahydropyrrolo[3,4-c]pyrrolyl, octahydro-1H-pyrido[1,2-a]pyrazinyl, 2-oxopiperazinyl, 2-oxopiperidinyl, 2-oxopyrrolidinyl, oxazolidinyl, 3,7-diazabicyclo[3.3.1]nonan-3-yl, piperidinyl, piperazinyl, 4-piperidonyl, pyrrolidinyl, pyrazolidinyl, quinuclidinyl, thiazolidinyl, tetrahydrofuranyl, thienyl[1,3]dithianyl, trithianyl, tetrahydropyranyl, thiomorpholinyl, thiamorpholinyl, 1-oxo-thiomorpholinyl, 1,1-dioxo-thiomorpholinyl, azetidinyl, octahydropyrrolo[3,4-c]pyrrolyl, octahydropyrrolo[3,4-b]pyrrolyl, decahydroprazino[1,2-a]azepinyl, azepanyl, azabicyclo[3.2.1]octyl, and 2,7-diazaspiro[4.4]nonanyl.

“Optionally substituted heterocyclyl” refers to a heterocyclyl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl, and each R²¹ is independently a direct bond or a straight or branched alkylene or alkenylene chain.

“N-heterocyclyl” refers to a heterocyclyl radical as defined above containing at least one nitrogen and where the point of attachment of the N-heterocyclyl radical to the rest of the molecule may be through a nitrogen atom in the N-heterocyclyl radical or through a carbon in the N-heterocyclyl radical.

“Optionally substituted N-heterocyclyl” refers to an N-heterocyclyl, as defined above, which is optionally substituted by one or more substituents as defined above for optionally substituted heterocyclyl.

“Heterocyclylalkyl” refers to a radical of the formula —R_{b}Rₕ where R_{b} is an alkylene chain as defined above and Rₕ is a heterocyclyl radical as defined above, and when the heterocyclyl is a nitrogen-containing heterocyclyl, the heterocyclyl may be attached to the alkylene chain at the nitrogen atom.

“Optionally substituted heterocyclylalkyl” refers to a heterocyclylalkyl radical, as defined above, wherein the alkylene chain of the heterocyclylalkyl radical is an optionally substituted alkylene chain, as defined above, and the heterocyclyl radical of the heterocyclylalkyl radical is an optionally substituted heterocyclyl radical, as defined above.

“Heterocyclylalkenyl” refers to a radical of the formula —R_{d}Rₕ where R_{d} is an alkenylene chain as defined above and Rₕ is a heterocyclyl radical as defined above, and when the heterocyclyl is a nitrogen-containing heterocyclyl, the heterocyclyl may be attached to the alkenylene chain at the nitrogen atom.

“Optionally substituted heterocyclylalkenyl” refers to a heterocyclylalkenyl radical, as defined above, wherein the alkenylene chain of the heterocyclylalkenyl radical is an optionally substituted alkenylene chain, as defined above, and the heterocyclyl radical of the heterocyclylalkenyl radical is an optionally substituted heterocyclyl radical, as defined above.

“Heterocyclylalkynyl” refers to a radical of the formula —RₑRₕ where Rₑ is an alkynylene chain as defined above and Rₕ is a heterocyclyl radical as defined above, and when the heterocyclyl is a nitrogen-containing heterocyclyl, the heterocyclyl may be attached to the alkynylene chain at the nitrogen atom.

“Optionally substituted heterocyclylalkynyl” refers to a heterocyclylalkynyl radical, as defined above, wherein the alkynylene chain of the heterocyclylalkynyl radical is an optionally substituted alkynylene chain, as defined above, and the heterocyclyl radical of the heterocyclylalkynyl radical is an optionally substituted heterocyclyl radical, as defined above.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. A heteroaryl radical is commonly, but not necessarily, attached to the parent molecule via an aromatic ring of the heteroaryl radical. For purposes of this disclosure, the heteroaryl radical may be a monocyclic, bicyclic or tricyclic ring system, which may include spiro or bridged ring systems; and the nitrogen, carbon or sulfur atoms in the heteroaryl radical may be optionally oxidized and the nitrogen atom may be optionally quaternized. For purposes of this disclosure, the aromatic ring of the heteroaryl radical need not contain a heteroatom, as long as one ring of the heteroaryl radical contains a heteroatom. For example benzo-fused heterocyclyls such as 1,2,3,4-tetrahydroisoquinolin-7-yl are considered a “heteroaryl” for the purposes of this disclosure. Except for the polycyclic heteroaryls containing more than 14 ring atoms, as defined below, a “heteroaryl” radical as defined herein can not contain rings having more than 7 members and cannot contain rings wherein two non-adjacent members thereof are connected through an atom or a group of atoms (i.e., a bridged ring system). Examples of heteroaryl radicals include, but are not limited to, azepinyl, acridinyl, benzimidazolyl, benzindolyl, 1,3-benzodioxolyl, benzofuranyl, benzoxazolyl, benzothiazolyl, benzothiadiazolyl, benzo[b][1,4]dioxepinyl, benzo[b][1,4]oxazinyl, benzo[b]azepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 3,4-dihydro-2H-benzo[b][1,4]dioxepinyl, cyclopenta[4,5]thieno[2,3-d]pyrimidinyl such as 6,7-dihydro-5H-cyclopenta[4,5]thieno[2,3-d]pyrimidinyl, 5,6-dihydrobenzo[h]quinazolinyl, 3,4-dihydro-2H-benzo[b][1,4]thiazinyl, 5,6-dihydrobenzo[h]cinnolinyl, 7′,8′-dihydro-5′H-spiro[[1,3]dioxolane-2,6′-quinoline]-3′-yl, 6,7-dihydro-5H-benzo[6,7]cyclohepta[1,2-c]pyridazinyl, 2,3-dihydro-1H-pyrido[2,3-b][1,4]oxazinyl, 3′,4′-dihydrospiro[cyclobutane-1,2′-pyrido[3,2-b][1,4]oxazinyl, dihydropyridooxazinyl such as 3,4-dihydro-2H-pyrido[3,2-b][1,4]oxazinyl, dihydropyridothiazinyl such as 3,4-dihydro-2H-pyrido[3,2-b][1,4]thiazinyl, dibenzofuranyl, dibenzothiophenyl, furanyl, furanonyl, furo[3,2-c]pyridinyl, furopyrimidinyl, furopyridazinyl, furopyrazinyl, isothiazolyl, imidazolyl, imidazopyrimidinyl, imidazopyridazinyl, imidazopyrazinyl, imidazo[1,2-a]pyridinyl, indazolyl, indolyl, indazolyl, isoindolyl, indolinyl, isoindolinyl, isoquinolinyl (isoquinolyl), indolizinyl, isoxazolyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 3′-oxo-3′,4′-dihydrospiro[cyclobutane-1,2′-pyrido[3,2-b][1,4]oxazine]yl, 7-oxo-5,6,7,8-tetrahydro-1,8-naphthyridinyl, 1-phenyl-1H-pyrrolyl, phenazinyl, phenothiazinyl, phenoxazinyl, phthalazinyl, phenanthridinyl, pteridinyl, purinyl, pyrrolyl, pyrazolyl, pyrazolo[3,4-d]pyrimidinyl, pyridinyl (pyridyl), pyrido[3,2-d]pyrimidinyl, pyrido[3,4-d]pyrimidinyl, pyrazinyl, pyrimidinyl, pyridazinyl (pyridazyl), pyrrolyl, pyrrolopyrimidinyl, pyrrolopyridazinyl, pyrrolopyrazinyl, 2H-pyrido[3,2-b][1,4]oxazinonyl, 1H-pyrido[2,3-b][1,4]oxazinonyl, pyrrolopyridinyl such as 1H-pyrrolo[2,3-b]pyridinyl, quinazolinyl, quinoxalinyl, quinolinyl, quinuclidinyl, tetrahydroquinolinyl, 5,6,7,8-tetrahydroquinazolinyl, 2,3,4,5-tetrahydrobenzo[b]oxepinyl, 6,7,8,9-tetrahydro-5H-cyclohepta[b]pyridinyl, 6,7,8,9-tetrahydro-5H-pyrido[3,2-c]azepinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, 1,2,3,4-tetrahydroisoquinolin-7-yl, triazinyl, thieno[2,3-d]pyrimidinyl, thienopyrimidinyl (e.g., thieno[3,2-d]pyrimidinyl), thieno[2,3-c]pyridinyl, thienopyridazinyl, thienopyrazinyl, and thiophenyl (thienyl).

“Optionally substituted heteroaryl” refers to a heteroaryl radical, as defined above, which is optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)₂R²⁰2, —R²¹—C(═NR²⁰)N(R²⁰)₂, —R²¹—S(O)ₜOR²⁰ (where t is 1 or 2), —R²¹—S(O)ₚR²⁰ (where p is 0, 1 or 2), and —R²¹—S(O)₂N(R²⁰)₂, where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, form an optionally substituted N-heterocyclyl or an optionally substituted N-heteroaryl, and each R²¹ is independently a direct bond or a straight or branched alkylene or alkenylene chain.

“N-heteroaryl” refers to a heteroaryl radical as defined above containing at least one nitrogen and where the point of attachment of the N-heteroaryl radical to the rest of the molecule may be through a nitrogen atom in the N-heteroaryl radical or through a carbon atom in the N-heteroaryl radical.

“Optionally substituted N-heteroaryl” refers to an N-heteroaryl, as defined above, which is optionally substituted by one or more substituents as defined above for optionally substituted heteroaryl.

“Polycyclic heteroaryl containing more than 14 ring atoms” refers to a 15- to 20-membered ring system radical comprising hydrogen atoms, one to fourteen carbon atoms, one to eight heteroatoms selected from the group consisting of nitrogen, oxygen and sulfur, and at least one aromatic ring. A “polycyclic heteroaryl containing more than 14 ring atoms” radical is commonly, but not necessarily, attached to the parent molecule via an aromatic ring of the “polycyclic heteroaryl containing more than 14 ring atoms” radical. For purposes of this disclosure, the “polycyclic heteroaryl containing more than 14 ring atoms” radical may be a bicyclic, tricyclic or tetracyclic ring system, which may include fused or spiro ring systems; and the nitrogen, carbon or sulfur atoms in the “polycyclic heteroaryl containing more than 14 ring atoms” radical may be optionally oxidized and the nitrogen atom may also be optionally quaternized. For purposes of this disclosure, the aromatic ring of the “polycyclic heteroaryl containing more than 14 ring atoms” radical need not contain a heteroatom, as long as one ring of the “polycyclic heteroaryl containing more than 14 ring atoms” radical contains a heteroatom. Examples of “polycyclic heteroaryl containing more than 14 ring atoms” radicals include, but are not limited to, 6,7-dihydro-5H-benzo[6,7]cyclohepta[1,2-c]pyridazin-3-yl, 6,7-dihydro-5H-pyrido[2′,3′:6,7]cyclohepta[1,2-c]pyridazin-3-yl, 6,7,8,9-tetrahydro-5H-cyclohepta[4,5]thieno[2,3-d]pyrimidin-4-yl, 6,7-dihydro-5H-benzo[6,7]cyclohepta[1,2-d]pyrimidin-4-yl, 6,7-dihydro-5H-benzo[2,3]azepino[4,5-c]pyridazin-3-yl, (Z)-dibenzo[b,f][1,4]thiazepin-11-yl, 6,7-dihydro-5H-benzo[6,7]cyclohepta[4,5-c]pyridazin-2-yl, 6,7-dihydro-5H-benzo[2,3]oxepino[4,5-c]pyridazin-3-yl, spiro[chromeno[4,3-c]pyridazine-5,1′-cyclopentane]-3-yl, 6,8,9,10-tetrahydro-5H-spiro[cycloocta[b]pyridine-7,2′-[1,3]dioxolane]-3-yl, 5,6,8,9-tetrahydrospiro[benzo[7]annulene-7,2′-[1,3]dioxolane]-3-yl, 5,7,8,9-tetrahydrospiro[cyclohepta[b]pyridine-6,2′-[1,3]dioxolane]-3-yl, 6,7-dihydro-5H-benzo[2,3]thiepino[4,5-c]pyridazin-3-yl, 6,7-dihydro-5H-benzo[6,7]cyclohepta[1,2-d]pyrimidin-2-yl, 5,6,8,9-tetrahydrospiro[cyclohepta[b]pyridine-7,2′-[1,3]dioxolane]-3-yl, 6,8,9,10-tetrahydro-5H-spiro[cycloocta[b]pyridine-7,2′-[1,3]dioxane]-3-yl and 6,7-dihydro-5H-benzo[6,7]cyclohepta[1,2-b]pyridin-2-yl.

“Optionally substituted polycyclic heteroaryl containing more than 14 ring atoms” is meant to include “polycyclic heteroaryl containing more than 14 ring atoms” radicals, as defined above, which are optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²⁰, —R²¹—N(R²⁰)C(O)R²⁰, —R²¹—N(R²⁰)S(O)ₜR²⁰ (where t is 1 or 2), —R²¹—S(O)ₜOR²⁰ (where t is 1 or 2), —R²¹—S(O)ₚR²⁰ (where p is 0, 1 or 2), and —R²¹—S(O)ₜN(R²⁰)₂ (where t is 1 or 2), where each R²⁰ is independently selected from the group consisting of hydrogen, alkyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl and optionally substituted heteroarylalkyl, or two R²⁰'s, together with the common nitrogen to which they are both attached, may optionally form an optionally substituted N-heterocyclyl or an optionally substituted N-heteroaryl, and each R²¹ is independently a direct bond or a straight or branched alkylene or alkenylene chain.

“Heteroarylalkyl” refers to a radical of the formula —R_{b}Rᵢ where R_{b} is an alkylene chain as defined above and Rᵢ is a heteroaryl radical as defined above, and when the heteroaryl is a nitrogen-containing heteroaryl, the heteroaryl may be attached to the alkylene chain at the nitrogen atom.

“Optionally substituted heteroarylalkyl” refers to a heteroarylalkyl radical, as defined above, wherein the alkylene chain of the heteroarylalkyl radical is an optionally substituted alkylene chain, as defined above, and the heteroaryl radical of the heteroarylalkyl radical is an optionally substituted heteroaryl radical, as defined above.

“Heteroarylalkenyl” refers to a radical of the formula —R_{d}Rᵢ where R_{d} is an alkenylene chain as defined above and Rᵢ is a heteroaryl radical as defined above, and when the heteroaryl is a nitrogen-containing heteroaryl, the heteroaryl may be attached to the alkenylene chain at the nitrogen atom.

“Optionally substituted heteroarylalkenyl” refers to a heteroarylalkenyl radical, as defined above, wherein the alkenylene chain of the heteroarylalkenyl radical is an optionally substituted alkenylene chain, as defined above, and the heteroaryl radical of the heteroarylalkenyl radical is an optionally substituted heteroaryl radical, as defined above.

“Heteroarylalkynyl” refers to a radical of the formula —RₑRᵢ where Rₑ is an alkynylene chain as defined above and Rᵢ is a heteroaryl radical as defined above, and when the heteroaryl is a nitrogen-containing heteroaryl, the heteroaryl may be attached to the alkynylene chain at the nitrogen atom.

“Optionally substituted heteroarylalkynyl” refers to a heteroarylalkynyl radical, as defined above, wherein the alkynylene chain of the heteroarylalkynyl radical is an optionally substituted alkynylene chain, as defined above, and the heteroaryl radical of the heteroarylalkynyl radical is an optionally substituted heteroaryl radical, as defined above.

“Hydroxyalkyl” refers to an alkyl radical as defined above which is substituted by one or more hydroxy radicals (—OH).

Certain chemical groups named herein may be preceded by a shorthand notation indicating the total number of carbon atoms that are to be found in the indicated chemical group. For example; C₇-C₁₂ alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

The compounds of formula (I), or their pharmaceutically acceptable salts, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, such as HPLC using a chiral column. When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present disclosure includes tautomers of any said compounds.

“Atropisomers” are stereoisomers resulting from hindered rotation about single bonds where the barrier to rotation is high enough to allow for the isolation of the conformers (Eliel, E. L.; Wilen, S. H. Stereochemistry of Organic Compounds; Wiley & Sons: New York, 1994; Chapter 14). Atropisomerism is significant because it introduces an element of chirality in the absence of stereogenic atoms. The disclosure is meant to encompass atropisomers, for example in cases of limited rotation around the single bonds emanating from the core triazole structure, atropisomers are also possible and are also specifically included in the compounds of the disclosure.

The chemical naming protocol and structure diagrams used herein are a modified form of the IUPAC nomenclature system wherein the compounds of formula (I) are named herein as derivatives of the central core structure, i.e., the triazole structure. For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency.

For purposes of this disclosure, the depiction of the bond attaching the R³ substituent to the parent triazole moiety in formula (I), as shown below:

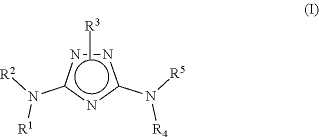

is intended to include only the two regioisomers shown below, i.e., compounds of formula (Ia) and (Ib):

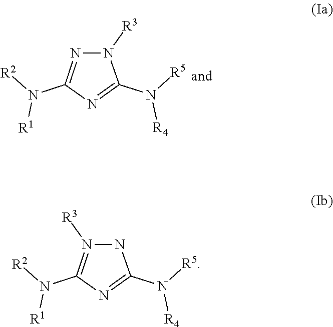

The numbering system of the ring atoms in compounds of formula (Ia) is shown below:

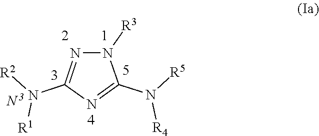

For example, a compound of formula (Ia) wherein R¹, R⁴ and R⁵ are each hydrogen, R² is 4-(2-(pyrrolidin-1-yl)ethoxy)phenyl and R³ is 6,7-dihydro-5H-benzo[6,7]cyclohepta[1,2-c]pyridazin-3-yl; i.e., a compound of the following formula:

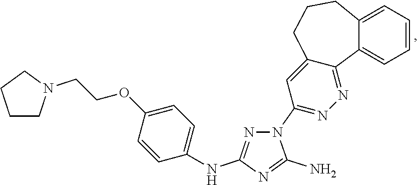 [SMILES: Nc1nc(Nc2ccc(OCCN3CCCC3)cc2)nn1-c1cc2c(nn1)-c1ccccc1CCC2]

is named herein as 1-(6,7-dihydro-5H-benzo[6,7]cyclohepta[1,2-c]pyridazin-3-yl)-N³-(4-(2-(pyrrolidin-1-yl)ethoxy)phenyl)-1H-1,2,4-triazole-3,5-diamine.

The numbering system of the ring atoms in compounds of formula (Ib) is shown below:

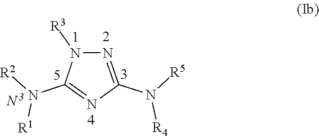

Compounds of formula (Ib) are similarly named herein.

### Antibody AXL Inhibitors

In some embodiments the AXLi is an antibody. Preferably the antibody AXL inhibitory activity. In some cases the antibody inhibits the binding of AXL to the GAS6 ligand.

In some embodiments, the anti-AXL antibody is an antibody as described in any of the following references: WO/2016/097370, WO/2017/220695, WO/2015/193428, WO/2016/166296, WO/2015/193430, EP2267454, WO/2009/063965, WO/2011/159980, WO/2012/175691, WO/2012/175692, WO/2013/064685, WO/2014/068139, WO/2009/062690, and WO/2010/130751 (the contents of each of which is hereby incorporated by reference).

In another embodiment, the anti-AXL antibody is an antibody as described in international patent application WO/2015/193428, the contents of which is hereby incorporated by reference, particularly as shown at pages 82-83.

In another embodiment, the anti-AXL antibody is an antibody as described in international patent application WO/2016/166296, the contents of which is hereby incorporated by reference, particularly the humanized 1H12 antibody disclosed therein.

In another embodiment, the anti-AXL antibody is an antibody as described in international patent application WO/2015/193430, the contents of which is hereby incorporated by reference, particularly as shown at pages 72-73.

In another embodiment, the anti-AXL antibody is an antibody as described in European patent publication EP2267454, the contents of which is hereby incorporated by reference.

In another embodiment, the anti-AXL antibody is an antibody as described in European patent publication WO/2009/063965, the contents of which is hereby incorporated by reference, particularly as shown at pages 31-33.

In another embodiment, the anti-AXL antibody is an antibody as described in US patent publication US 2012/0121587 A1, the contents of which is hereby incorporated by reference, particularly as shown at pages 26-61.

In another embodiment, the anti-AXL antibody is an antibody as described in international patent publication WO/2011/159980, the contents of which is hereby incorporated by reference, particularly the YW327.6S2 antibody as shown in FIG. 2, Figure page 6 (of 24).

In another embodiment, the anti-AXL antibody is an antibody as described in international patent publication WO/2012/175691, the contents of which is hereby incorporated by reference, particularly as shown at page 5.

In another embodiment, the anti-AXL antibody is an antibody as described in international patent publication WO/2012/175692, the contents of which is hereby incorporated by reference, particularly as shown at pages 4-5.

In another embodiment, the anti-AXL antibody is an antibody as described in international patent publication WO/2009/062690, the contents of which is hereby incorporated by reference.

In another embodiment, the anti-AXL antibody is an antibody as described in international patent publication WO/2010/130751, the contents of which is hereby incorporated by reference, particularly as shown at pages 1-17 (of 78).

In another embodiment, the anti-AXL antibody is an antibody as described in international patent publication WO/2013/064685, the contents of which is hereby incorporated by reference, particularly the 1613F12 antibody described therein as shown at, for example, Examples 6 to 8.

In another embodiment, the anti-AXL antibody is an antibody as described in international patent publication WO/2014/068139, the contents of which is hereby incorporated by reference, particularly the 110D7, 1003A2, and 1024G11 antibodies described therein as shown at, for example, Examples 6 to 8.

In another embodiment, the anti-AXL antibody is an antibody as described in international patent publication WO/2016/097370, the contents of which is hereby incorporated by reference, particularly the murine 10G5 and 10C9 antibodies described therein as shown at, for example, Examples 6 to 8.

In another embodiment, the anti-AXL antibody is an antibody as described in international patent publication WO/2017/220695, the contents of which is hereby incorporated by reference, particularly the humanized 10G5 antibody described therein as shown at, for example, SEQ ID NO. 1 to 10.

### Preferred Embodiments

Preferably, the anti-AXL antibody is an antibody as described in WO/2016/097370, WO/2017/220695, WO/2015/193428, WO/2016/166296, WO/2015/193430, WO/2011/159980, WO/2013/064685, or WO/2014/068139 (the contents of each of which is hereby incorporated by reference).

More preferably, the anti-AXL antibody is an antibody as described in WO/2016/097370, WO/2017/220695, WO/2011/159980, WO/2013/064685, or WO/2014/068139 (the contents of each of which is hereby incorporated by reference).

Most preferably the anti-AXL antibody is an antibody as described in WO/2017/220695, particularly the humanized 10G5 antibody described therein as shown at, for example, Examples 6 to 8.

In some embodiments, the anti-AXL antibody comprises the 6 CDRs having the sequences set out herein in SEQ ID Nos. 1 to 6.

In some embodiments, the anti-AXL antibody comprises the 6 CDRs having the sequences set out herein in SEQ ID Nos. 7 to 12.

In some embodiments, the anti-AXL antibody comprises a VH domain having the sequence set out herein in either one of SEQ ID Nos. 13 or 14. In some embodiments the antibody further comprises a VL domain having the sequence set out herein in either one of SEQ ID Nos. 15 or 16.

### Antiviral Agents

In some embodiments, the AXLi described herein are administered in combination with one or more “second antiviral agents” or “second antiviral compounds”. Typically these agents and compounds act on the viral load (also called infectious or viral titre) by inhibiting either directly or indirectly the replication and/or dissemination of the virus infection within an infected subject organism.

Typically “antiviral activity” or “antiviral action” indicates an action on the virus or on its target cells, in particular the action of inhibiting the replication cycle of the virus or its ability to infect and to be reproduced in host cells, wherein this antiviral effect can be obtained by modulating a number of genes of the target cells (cells infected with the avirus and/or likely to be infected in the near future, because of their close proximity with infected cells).

In some embodiments the second antiviral agent is selected from the pharmaceutical classes of agents disclosed in international application WO2015/157223. For example, in some embodiments the second antiviral agent is selected from: antibacterial agents, antiparasitic agents, neurotransmission inhibitors, estrogen receptor inhibitors, DNA synthesis and replication inhibitors, protein maturation inhibitors, kinase pathway inhibitors, cytoskeleton inhibitors, lipid metabolism inhibitors, anti-inflammatory agents, ion cham1el inhibitors, apoptosis inhibitors, and cathepsin inhibitors.

Preferably, an antiviral agent acts on a virus to inhibit and/or slow and/or prevent the associated viral infection. Antiviral agents are classified in different categories depending on their mode of action. These include in particular that are of use in the present methods:
nucleotide analogues, which interfere or stop DNA or RNA synthesis; as well as inhibitors of the enzymes involved in DNA or RNA synthesis (helicase, replicase);
compounds which inhibit the virus maturation steps during its replication cycle;
compounds which interfere with cell membrane binding, or virus entry in host cells (fusion or entry inhibitors);
agents which prevent the virus from being expressed within the host cell after its entry, by blocking its disassembly within the cell;
agents which restrict virus propagation to other cells.

In some embodiments, the second antiviral agent is one of those well known in the art. For example: ribavirin, a guanosine nucleoside analogue with a wide antiviral spectrum; and/or members of the three interferon families, alpha, beta and gamma. For example, the efficiency of interferon alpha-2b to inhibit the in vivo and in vitro replication of viruses has been demonstrated.

### Other Combinations

In some cases the second antiviral agent is remdesivir.

In some embodiments, the AXLi is administered in combination with an anti-inflammatory agent. The anti-inflammatory agent may be corticosteroid or a glucocorticoid steroid such as dexamethasone.

In some embodiments, the AXLi is administered in combination with an immunosuppressive agent. The immunosuppressive agent may be an IL-6 anatgonist such as Tocilizumab.

### Definitions

“A virus infection” and corresponding terms as used herein mean that the subject organism has cells that have been infected by the named virus class or type. The infection can in particular be established by performing a detection and/or viral titration from respiratory samples, or by assaying virus-specific blood-circulating antibodies. The detection in the individuals infected with the specific virus may be made by conventional diagnostic methods, in particular of molecular biology (PCR), which are well known to those skilled in the art.

The term “treatment/treating” indicates fighting the virus infection in the subject organism. Typically, administration of the AXLi according to the present disclosure will lead to a decrease of the viral infection rate (infectious titre) in the subject, preferably to non-pathological levels (eventually to undetectable levels). In some embodiments, the administration of the AXLi leads to an at least a 10% decrease in viral titre as compared to an otherwise comparable control subject that has not received the AXLi. For example, in some embodiments administration of the AXLi leads to an at least 20% reduction in viral title, such as an at least 30%, at least 40%, at least 50%, at least 60%, at least 70%, at least 80%, at least 90%, at least 95%, or at least 99% reduction in viral titre.

In some cases the methods of treatment disclosed herein result in improved survival of subjects receiving an AXLi as compared to otherwise comparable subjects not receiving the AXLi. In some cases the survival is measured as the percentage of subjects surviving at a particular time point after the start of AXLi administration, for example 14, 28, 42, or 56 days after the start of AXLi administration. So, for example, if from a population of 90 subjects receiving treatment 3 died by the elected timepoint, % survival=87/90=96.7%. In comparison, if from a population of 90 subjects receiving treatment 10 died by the elected timepoint, % survival=80/90=88.9%. In some cases the improvement in survival at the selected timepoint is at least 2%, such as at least 3%, at least 4%, at least 5%, at least 6%, at least 7%, at least 8%, at least 9%, or at least 10%.

In some cases the methods of treatment disclosed herein result in improved viral clearance from subjects receiving an AXLi as compared to otherwise comparable subjects not receiving the AXLi. In some cases viral clearance is measured as the percentage of subjects having undetectable levels (ie. below the LLoQ) of salivary virus as measured by the assay set out herein in Example 10 at a particular time point after the start of AXLi administration, for example 1, 3, 5, 8, 11, 15, or 29 days after the start of AXLi administration. In some cases the improvement in survival at the selected timepoint is at least 10%, such as at least 20%, at least 30%, at least 40%, or at least 50%.

The term “treatment/treating” is also used herein to indicate the attenuation of symptoms associated with the viral infection. For example, a reduction in the level of fever experienced by the subject, or an improvement in blood oxygenation. In some embodiments, administration of the AXLi reduces the subject's temperature by at least 0.1 C within 24 hours of administration of the AXLi. For example, in some embodiments, administration of the AXLi reduces the subject's temperature by at least 0.2 C, such as at least 0.3 C, at least 0.4 C, at least 0.5 C, at least 0.8 C, at least 1.0 C, at least 1.5 C, or at least 2.0 C within 24 hours of administration of the AXLi. In some embodiments, administration of the AXLi increases the blood oxygenation of the subject by at least 1% within 24 hours of administration of the AXLi. For example, in some embodiments, administration of the AXLi increases the blood oxygenation of the subject by at least 2%, such as at least 3%, at least 4%, at least 5%, at least 8%, at least 10%, at least 15%, or at least 20% within 24 hours of administration of the AXLi.

As used herein, the term “prevention/preventing” indicates stopping, or at least decreasing the probability of occurrence of an infection in subject organism by the virus. In some embodiments, administration of the AXLi leads to the cells of the subject organism to be less receptive to infection by the virus and are thus less likely to be infected.

As used herein “efficient amount” means an amount sufficient to inhibit the proliferation and/or replication of the virus, and/or the development of the viral infection within the subject organism. This inhibition can be measured by, for example, measuring the viral titre in the subject, as illustrated in Example 1.

As used herein, the term “mutation” is used to indicate a change in a nucleotide or amino acid sequence relative to a reference (eg. wild type, or original) sequence. Typically, in the context of the SARS-CoV-2 “mutations” discussed herein, the changes are relative to the sequence of the Wuhan-Hu-1 strain. Unless context clearly indicates otherwise mutations may by substitutions, deletions, or insertions. In this regard, substitutions are typically indicated by the nomenclature ‘X123Y’, where X is the wild-type identity, 123 is the sequence position, and Y is the the mutant identity. Similarly, a Greek delta symbol (‘Δ’) is typically used to indicate a deletion at the position number it immediately precedes.

### Subject Selection

In certain aspects, the subjects are selected as suitable for treatment with the treatments before the treatments are administered.

As used herein, subjects who are considered suitable for treatment are those subjects who are expected to benefit from, or respond to, the treatment.

Subjects may have, or be suspected of having, or be at risk of having a viral infection and/or at particular risk of severe symptoms if they were to catch the viral infection.

Thus, in some embodiments a subject is selected for treatment if they are a member of a group having, or expected to have, high levels of exposure to the virus. For example, in some embodiments the subject is a healthcare professional, such as a doctor or a nurse. In some embodiments the subject is a key worker, such as a pharmacist, police officer, or work in food provision.

In some embodiments the subject has, is suspected of having, or is at risk of having, one or more comorbidity that increases the risk of experiencing severe symptoms or death if infected with the virus. In some embodiments the subject has, is suspected of having, or is at risk of having, one or more comorbidity selected from: respiratory system disease (such as COPD or asthma), cardiovascular disease (such as congestive heart failure), diabetes, hypertension, cancer, or a suppressed immune system (such as a transplant recipient).

It has also been noted that some of the viral infections discussed herein result in notably more severe symptoms in older subjects, particularly older male subjects. Accordingly, in some embodiments the subject is selected for treatment with the AXLi if they are at least 50 years old, for example, at least 60 years old, at least 70 years old, or at least 80 years old. In some embodiments the subject is selected for treatment if they are male.

In some aspects, the subject is selected as suitable for treatment due to the level of marker expression in a sample. Depending on the specific marker(s) tested, subjects with or without marker may be considered suitable for treatment.

In other aspects, the level of marker expression is used to select a subject as suitable for treatment. In some cases, depending on the specific marker(s) tested, where the level of expression of the marker is increased or decreased relative to a control the subject is determined to be suitable for treatment.

In some aspects, the presence of a marker or combination of markers in the sample indicates that the subject is suitable for treatment with the methods described herein. In other aspects, the amount of a marker or combination of markers must be increased or decreased relative to a control to indicate that the subject is suitable for treatment. In some aspects, the observation that a marker's localisation is altered in the sample as compared to a control indicates that the subject is suitable for treatment.

In some cases the subject is selected for treatment based on the subject's level of C-reactive protein (CRP). The CRP level may be measured in a blood sample. In some cases the subject is selected for treatment if their CRP level is at least 10 μg/mL, at least 15 μg/mL, such as at least 20 μg/mL, at least 25 μg/mL, at least 30 μg/mL, at least 35 μg/mL, at least 40 μg/mL, at least 45 μg/mL, at least 50 μg/mL, at least 55 μg/mL, at least 60 μg/mL, at least 65 μg/mL, at least 70 μg/mL, at least 75 μg/mL, at least 80 μg/mL, at least 85 μg/mL, at least 90 μg/mL, at least 95 μg/mL, or at least 100 μg/mL. In some cases the subject is selected for treatment if their CRP level is at least 30 μg/mL. In some cases the subject is selected for treatment if their CRP level is at least 50 μg/mL.

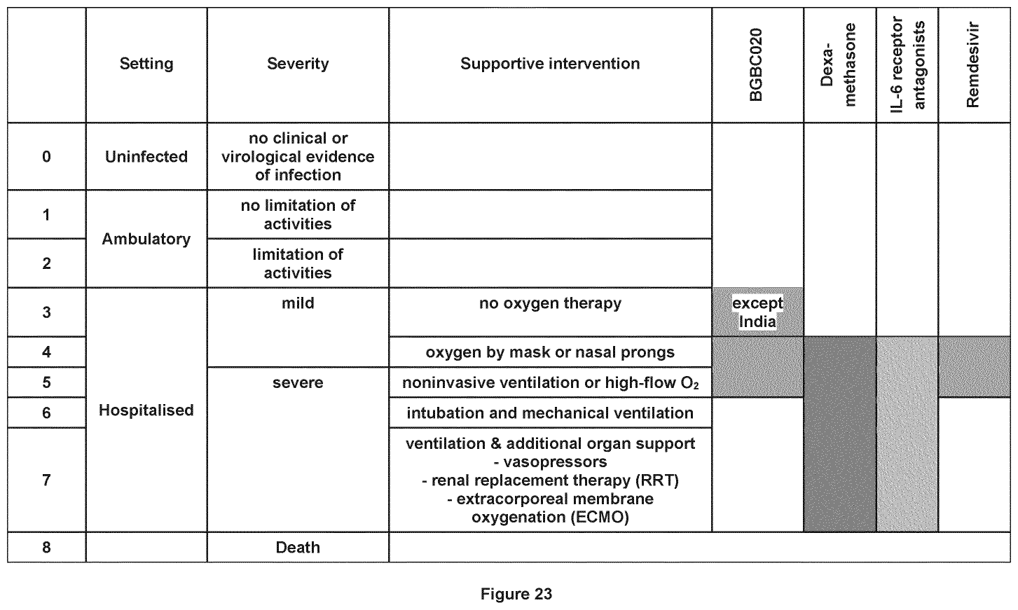

In some cases the subject is selected for treatment if the subject is at either level 4 or level 5 of the WHO COVID-19 9-point ordinal category scale (OCS) as shown in FIG. 23.

### Samples

The sample may comprise or may be derived from: a quantity of blood; a quantity of serum derived from the subject's blood which may comprise the fluid portion of the blood obtained after removal of the fibrin clot and blood cells; a quantity of pancreatic juice; a tissue sample or biopsy; or cells isolated from said subject.

A sample may be taken from any tissue or bodily fluid. In certain aspects, the sample may include or may be derived from a tissue sample, biopsy, resection or isolated cells from said subject.

In certain aspects, the sample is a tissue sample

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a subject's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a marker polypeptide or nucleic acid in the blood, in white blood cells, peripheral blood mononuclear cells, granulocytes and/or red blood cells.

The sample may be fresh or archival. For example, archival tissue may be from the first diagnosis of a subject, or a biopsy at a relapse. In certain aspects, the sample is a fresh biopsy.

### Subject Status

The subject may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human. In preferred embodiments, the subject is a human.

Furthermore, the subject may be any of its forms of development, for example, a foetus.

The terms “subject”, “patient” and “individual” are used interchangeably herein.

In some cases the subject has, is suspected of having, or has received a diagnosis of, a virus infection.

In some aspects disclosed herein, an subject has, or is suspected as having, or has been identified as being at risk of, or has received a diagnosis of an immune disorder, cardiovascular disorder, thrombosis, diabetes, immune checkpoint disorder, or fibrotic disorder (fibrosis) such as strabmisus, scleroderma, keloid, Nephrogenic systemic fibrosis, pulmonary fibrosis, idiopathic pulmonary fibrosis (IPF), cystic fibrosis (CF), systemic sclerosis, cardiac fibrosis, non-alcoholic steatohepatitis (NASH), other types of liver fibrosis, primary biliary cirrhosis, renal fibrosis, cancer, and atherosclerosis.

### Controls

In some aspects, target expression in the subject is compared to target expression in a control. Controls are useful to support the validity of staining, and to identify experimental artefacts.

Preferably, the control is a sample from a comparable neoplastic disorder that is not characterized by the presence of cells having a persister-cell phenotype, as defined by one or more of the features described herein.

The control may be a reference sample or reference dataset. The reference may be a sample that has been previously obtained from a subject with a known degree of suitability. The reference may be a dataset obtained from analyzing a reference sample.

Controls may be positive controls in which the marker(s) is known to be present, or expressed at known level, or negative controls in which the target molecule is known to be absent or expressed at low level.

Controls may be samples of tissue that are from subjects who are known to benefit from the treatment. The tissue may be of the same type as the sample being tested. For example, a sample of tumor tissue from a subject may be compared to a control sample of tumor tissue from a subject who is known to be suitable for the treatment, such as a subject who has previously responded to the treatment.

In some cases the control may be a sample obtained from the same subject as the test sample. The test and control samples may be collected at the same time from, for example, different tissues or locations in the same tissue. Alternatively, the test sample and control sample may be from the same or similar tissue or location, but taken at different times

In some cases, the control is a cell culture sample.

In some cases the control sample is a sample collected from the subject after treatment with an AXLi as disclosed herein.

In some cases, a test sample is analyzed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern. For example, where the amount of a first target protein and/or a second target protein expression is analyzed, the method may involve confirmation that in the test sample the expression is observed as membrane staining, with a cytoplasmic component. The method may involve confirmation that the ratio of target signal to noise is above a threshold level, thereby allowing clear discrimination between specific and non-specific background signals.

### Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

Typically, in the methods of treatment described herein the agents (eg. AXLi) are administered in a therapeutically or prophylactically effective amount.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Typically, the subjects treated are in need of the described treatment.

Disclosed herein are methods of therapy. Also provided is a method of treatment, comprising administering to a subject in need of treatment a therapeutically-effective amount of an AXLi. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The treatment may involve administration of the AXLi alone or in further combination with other treatments, either simultaneously or sequentially dependent upon the condition to be treated.

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

In some embodiments of the methods of treatment described herein, the AXLi is comprised in a pharmaceutical composition, optionally further comprising a pharmaceutically acceptable excipient.

### Dosage

It will be appreciated by one of skill in the art that appropriate dosages of the AXLi and compositions comprising the active element, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of AXLi is determined by the expression of a first marker observed in a sample obtained from the subject. Thus, the level or localisation of expression of the first marker in the sample may be indicative that a higher or lower dose of AXLi is required. For example, a high expression level of the first marker may indicate that a higher dose of AXLi would be suitable. In some cases, a high expression level of the first marker may indicate the need for administration of another agent in addition to the AXLi. For example, administration of the AXLi in conjunction with a second agent. A high expression level of the first marker may indicate a more aggressive therapy.

In certain aspects, the dosage level is determined by the expression of a first target protein, such as AXL, on cells in a sample obtained from the subject. For example, when the target neoplasm is composed of, or comprises, neoplastic cells expressing the first target protein.

In certain aspects, the dosage level is determined by the expression of a first target protein, such as AXL, on cells associated with the target tissue.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, each active compound is administered to a human subject according to the following dosage regime: about 100 mg, 3 times daily.

In one embodiment, each active compound is administered to a human subject according to the following dosage regime: about 150 mg, 2 times daily.

In one embodiment, each active compound is administered to a human subject according to the following dosage regime: about 200 mg, 2 times daily.

However in one embodiment, each conjugate compound is administered to a human subject according to the following dosage regime: about 50 or about 75 mg, 3 or 4 times daily.

In one embodiment, each conjugate compound is administered to a human subject according to the following dosage regime: about 100 or about 125 mg, 2 times daily.

### Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2 ml, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and p, respectively. The subunit structures and three-dimensional configurations of different classes of immunoglobulins are well known.

### BRIEF DESCRIPTION OF THE FIGURES

Embodiments and experiments illustrating the principles of the disclosure will now be discussed with reference to the accompanying figures in which: FIG. 1.
AXL promotes viral infection by two different mechanisms
(A) Facilitation of virus attachment and entry. Enveloped viruses display phosphatidylserine (PS) that is recognized by the GLA domain of GAS6. GAS6, which has high affinity (30 pM) for the AXL extracellular domain, facilitates virus attachment to cells by binding the AXL receptor (1). The tethered viral particle can enter the cell by phagocytosis, mimicking normal efferocytosis of apoptotic cell bodies (2).
(B) Suppression of type I interferon response. Viral particle binding of AXL also promotes receptor activation, which through an interaction with the type I IFN receptor (IFNAR), induces expression of SOCS1 and SOCS3, key negative regulators of IFN and cytokine signaling. AXL signaling induced response to viral infection results in the decreased expression of several genes associated with type I IFN production, thereby blunting the innate antiviral response and promoting virus replication. The selective AXL kinase inhibitor bemcentinib blocks AXL receptor activation and signaling required for both of these mechanisms, resulting in reduced viral entry and replication.

FIG. 2.
Summary means of virus load (CT) relative to HPRT present in cells following the addition of 0.1 ul or 0.01 ul or 0.001 ul of virus solution in the presence (“BGB” columns) or absence (“WT” or “KO” columns) of 1 μM Bemcemtinib (see Example 1)
   (A) Wild-type cells
   (B) ISG15 Knock-out cells

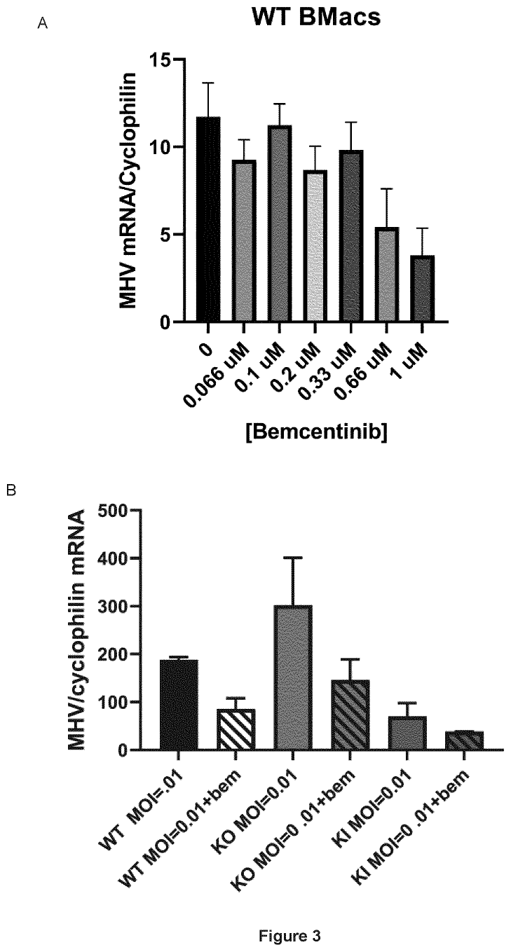

FIG. 3.
Analysis of the effect of bemcentinib on mouse betacoronavirus (mouse hepatitis virus, MHV) infection of mouse bone marrow-derived macrophages (mBMDM) in vitro (see Example 4)
   (A) Bemcentinib inhibits MHV infection of murine bone marrow macrophages (mBMDMs). mBMDMs were isolated from a femur, adhered to tissue culture plates and incubated in the presence of 20 ng/ml of murine macrophage colony stimulating factor (MCSF) for 6 days to allow maturation. Replicate wells of ˜250,000 cells in a 96-well format were infected with a MOI of 0.01. 24 h following infection, cells were washed and RNA isolated. Levels of the MHV spike gene and the housekeeping gene, Cyclophilin, were assessed by qRT-PCR. n=3
   (B) Bemcentinib reduces virus load in WT BMDMs. Cells were pretreated with 1 pM bemcentinib and the drug remained on the cells throughout the infection. RNA from infected cells was harvested at 24 h of infection and virus load was determined by qRT-PCR. (Not sure the housekeeping gene was cyclophilin). While the reduction of virus load in the other cells was not statistically significant, a trend towards reduction of virus load in the presence of bemcentinib was consistently observed.

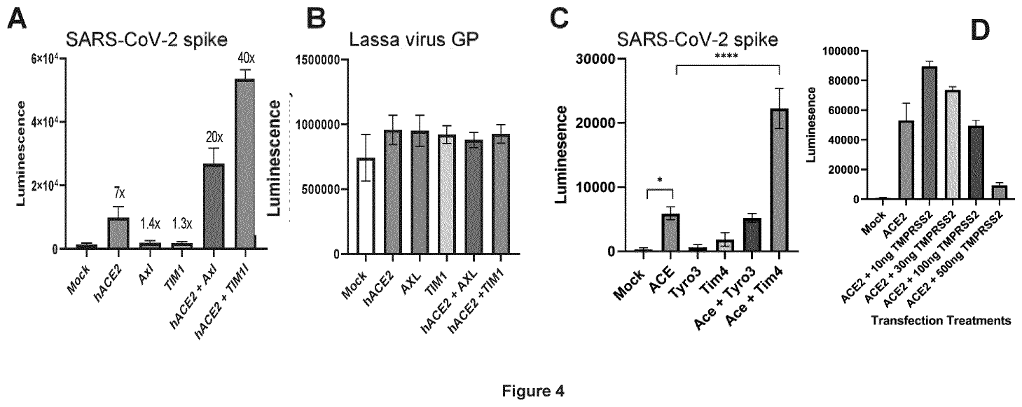

FIG. 4.
VSV pseudovirion-luciferase infections of HEK 293T cells transfected with entry factors by themselves or in combination with hACE2. At 48 h following transfection, virus was added to cells for 24 when cells were lyzed and luciferase activity was determined. A-B) Axl, and TIM-1 synergize with hACE2 to enhance SARS-CoV-2 spike dependent entry (A), but not Lassa virus GP dependent entry (B). C) TIM-4 enhances SARS-CoV-2 spike dependent entry into HEK 293T cells, but Tyro3 had no effect. D) Low, but not high, concentrations of TMPRSS2 plasmid enhances hACE2 dependent entry of SARS-CoV-2.

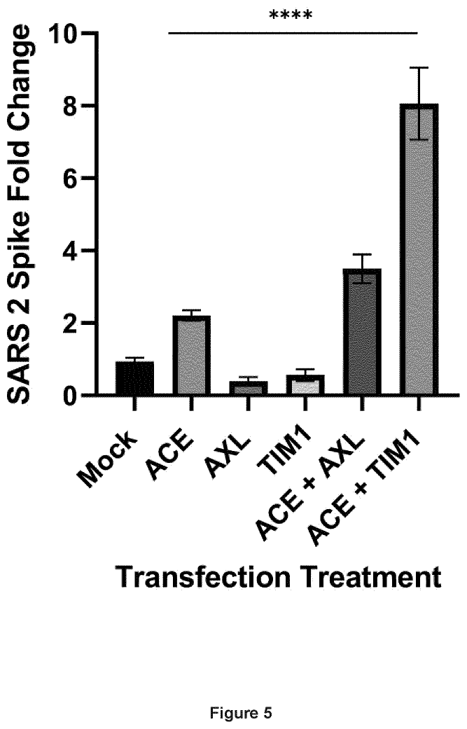

FIG. 5
HEK 293T cells transfected with low levels of hACE2 as well as AXL or TIM-1 and infected with SARS-CoV-2 at 48 h following transfection. RNA was harvested from infected cells at 24 h. Shown are qRT-PCR studies normalized to infection in the absence of hACE2. Virus expression levels were normalized to the housekeeping gene GAPDH.

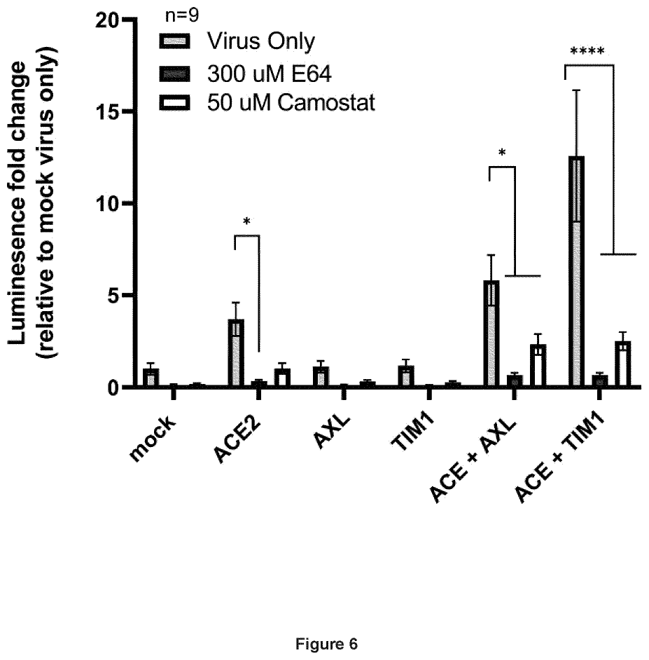

FIG. 6
Inhibition of hACE2-dependent VSV/SARS-CoV-2 spike pseudovirion infection by E64 or camostat in HEK 293T cells. Plasmids expressing the various receptors were transfected as shown in FIG. 3. Twenty-four h later, cells were infected with equivalent amounts of VSV-luciferase/SARS-COV-2 spike pseudovirions in the presence or absence of E64 or camostat. Luminescence was assessed 24 h later.

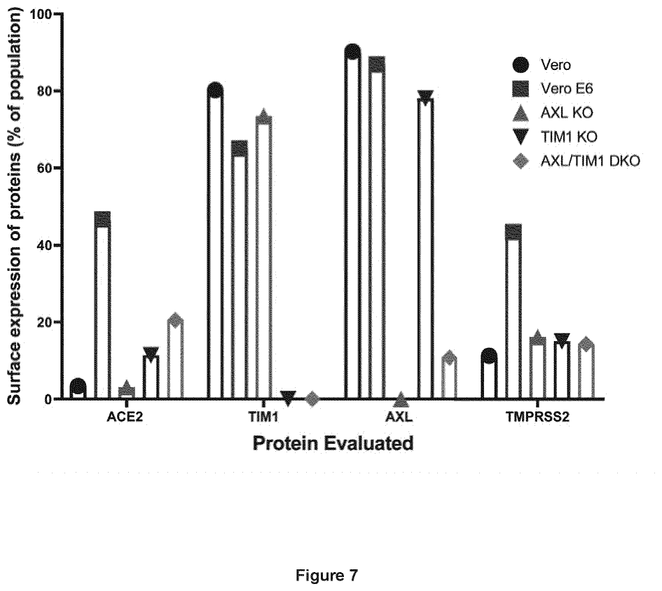

FIG. 7
Endogenous surface expression of proteins relevant to SARS-CoV-2 entry. Cells were lifted by EDTA and surface expression was detected with appropriate primary antibodies followed by Alexa 647-conjugated secondary antibodies and flow cytometry.

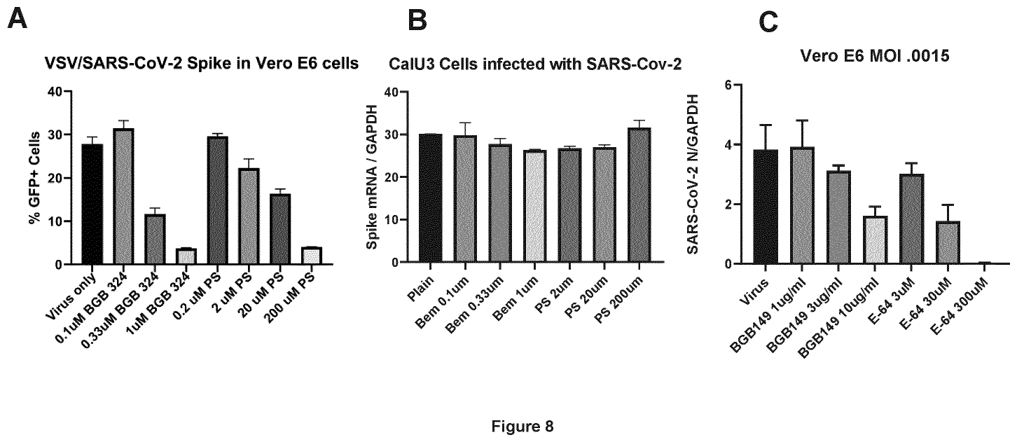

FIG. 8
Inhibition of SARS-Cov-2-spike dependent entry. A) Infection of VSV/SARS-CoV-2-GFP pseudovirions of Vero E6 cells in the presence of bemcentinib or PS liposomes at the doses shown. GFP expression was assessed at 24 h by flow cytometry. Sufficient virus to achieve infection of ˜30% of the “virus only” treated cells was added to cultures as this is in the linear portion of the infectivity curve. B and C) Viral load of WT SARS-CoV-2 infection of CalU3 cells (MOI=5) (B) or Vero E6 (MOI=0.0015) (C) at 24 h in the presence of the inhibitors noted in the panels. More virus was added to the CalU3 cells as those cells are relatively poorly permissive for SARS-CoV-2 infection. A range of MOIs were tested in the Vero E6 cells and similar findings were observed.

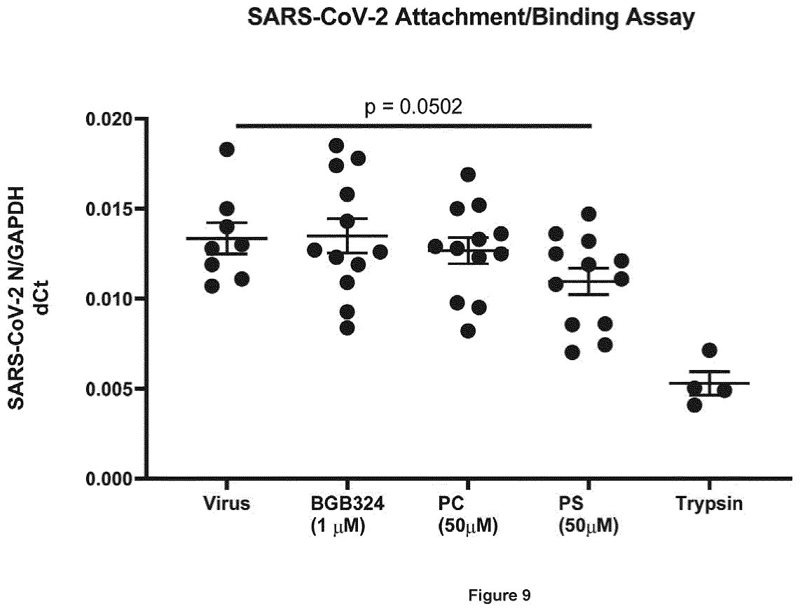

FIG. 9
Evaluation of SARS-CoV-2 binding on Vero E6 cells. Cells were incubated with virus (MOI=5) and treatments noted at 15° C. for 60 m. Subsequently, some cells were treated with trypsin to remove bound virus. All cells were washed extensively to remove unbound virus and cells were extracted for RNA. Viral load was determined and normalized for the host gene GAPDH. This is one of 3 different experiments yielding similar results.

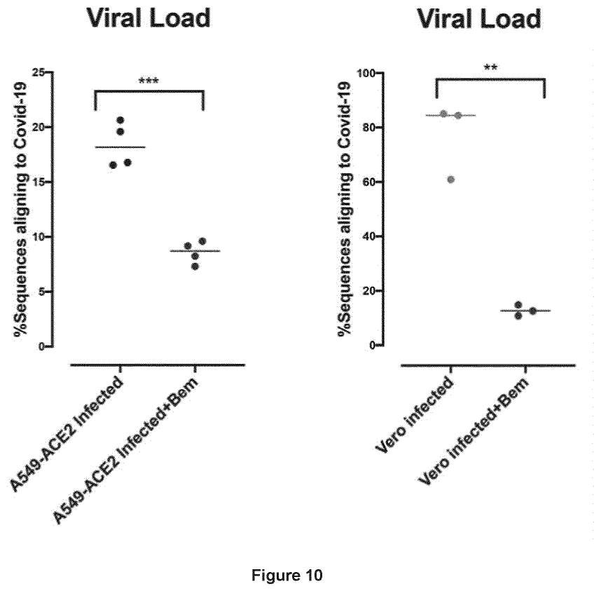

FIG. 10
MHV transcripts in A549-hACE2 cells (left) and Vero E6 cells (right) is significantly reduced. Cells were infected in the presence or absence of 1 uM Bemcentinib and infected with SARS-CoV-2 for 24 h. RNA was isolated and RNAseq was performed.

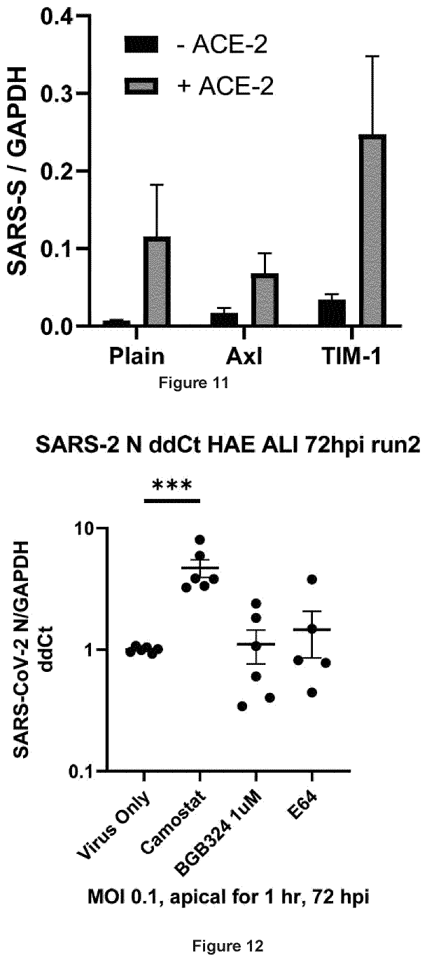

FIG. 11
Ability of 6-day macrophage colony stimulating factor matured hMDMs to support SARS-COV-2 infection. Infection was performed in the absence or presence of hACE2. In addition, human AXL or TIM-1 was expressed in some cells. Ad5 virus vectors (MOI=˜50) delivered the human genes. N=8-16 (single experiment)

FIG. 12
Ability of SARS-CoV-2 to infect HAE cultures in the presence of camostat, bemcentinib or E64. Shown are duplicate cultures from HAEs from 3 different donors.

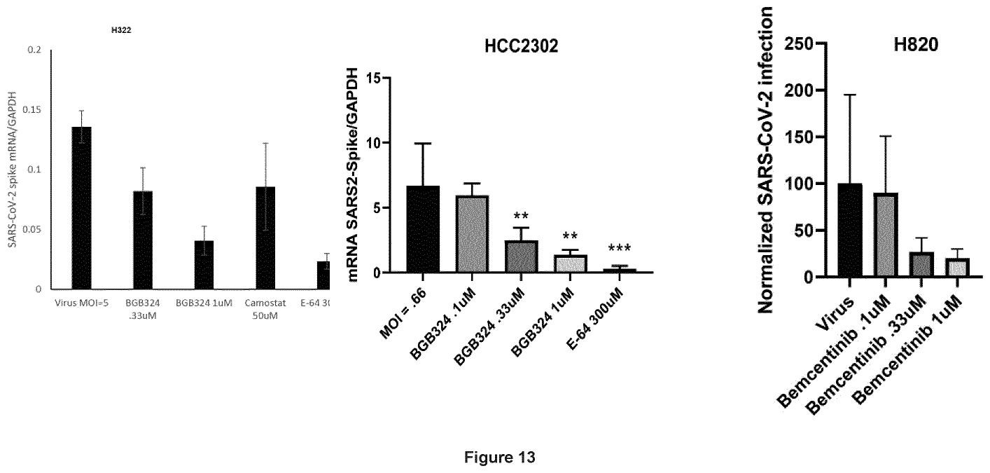

FIG. 13
Representative data from three of the lung tumor lines from John Minna that support SARS-COV-2 infection which is sensitive to both E64 and bemcentinib.

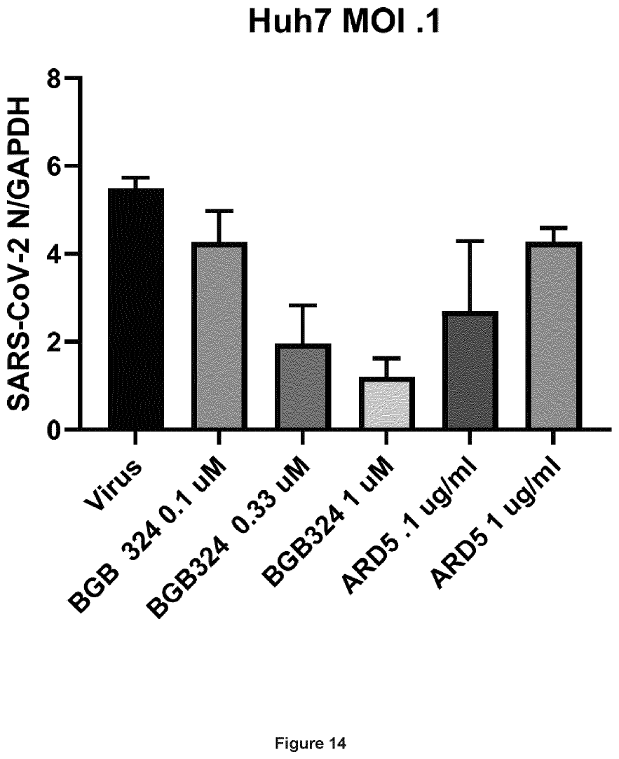

FIG. 14
Ability of PS receptor inhibitors to decrease SARS-Cov-2 infection of Huh7 cells.

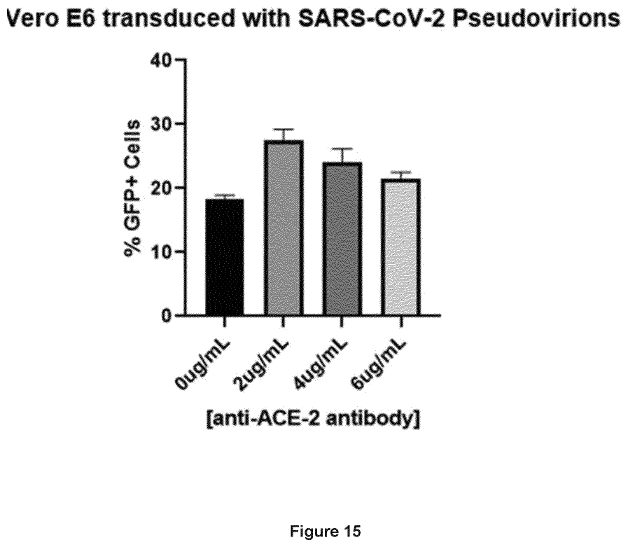

FIG. 15
Polyclonal sera against hACE2 does not inhibit SARS-CoV-2 spike-dependent infection. Antisera concentrations noted above were added ˜1 h prior to infection and maintained in the culture for the 24 h infection period.

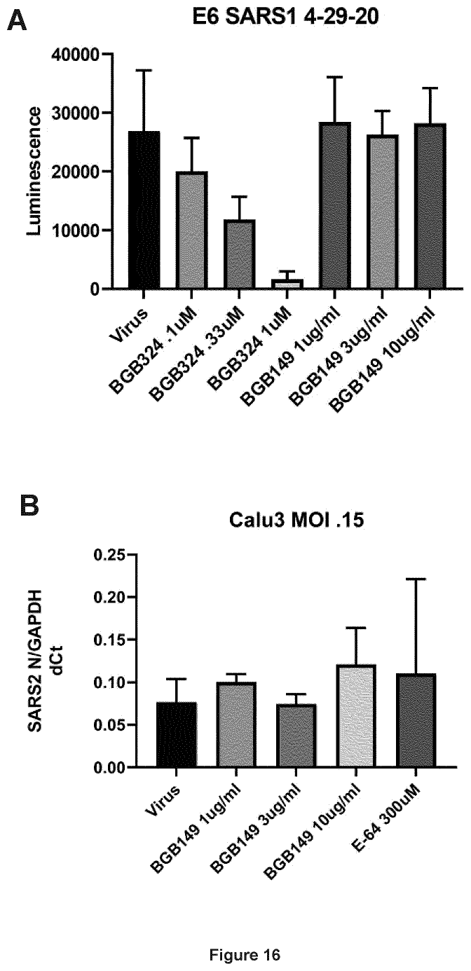

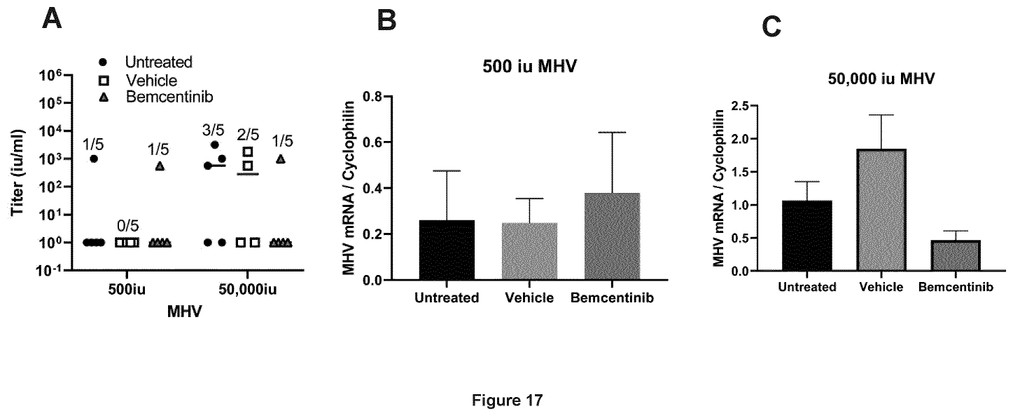

FIG. 16
(A) Vero E6 cell studies where it was observed that bemcentinib was highly effective at inhibiting SARS-CoV-2 spike dependent entry, but tilvestamab had no effect. VSV/SARS-CoV-2 spike pseudovirions were used this experiment.
(B) Tilvestamab has no effect on SARS-CoV-2 infection of CalU3 cells.

FIG. 17
MHV present in livers of infected C57BL/6 mice at day 3 of infection. Virus was administered intraperitoneally. Virus was assessed by titering clarified liver homogenates on a murine CEACAM+ cell line (A) or by qRT-PCR (B-C). No statistical significance is achieved when treatments are grouped into n=5.

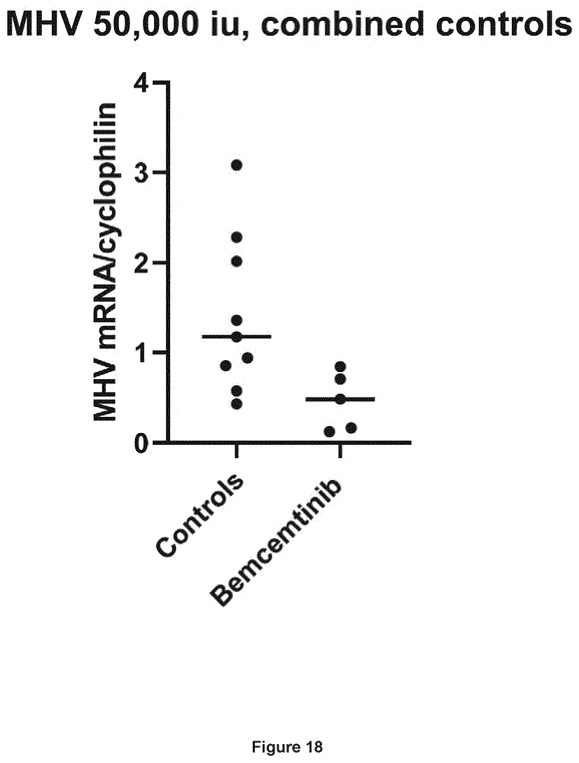

FIG. 18
Virus load at day X of infection with 50,000 iu of MHV. Same data as shown in FIG. 18C. However, untreated and vehicle only groups of mice given 50,000 iu of MHV are now pooled. When compared in a Student's t test, the bemcentinib group now has a significantly reduced virus load.

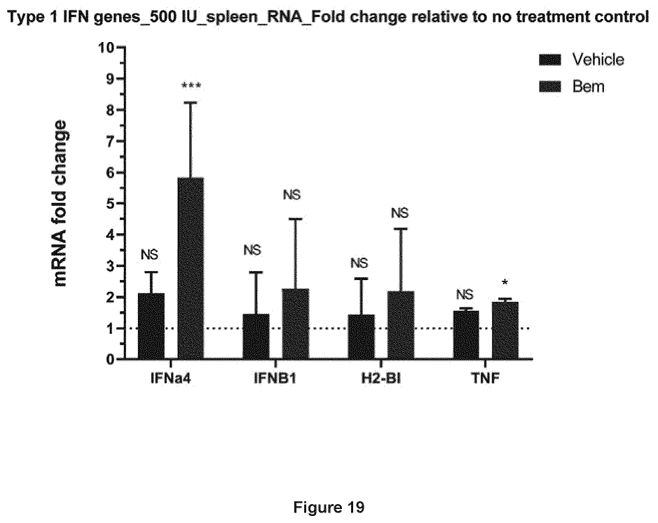

FIG. 19
Expression changes in interferon related genes at day 3 in spleens of C57BL/6 mice infected with 500 iu of MHV in the presence of vehicle control (LH bar in each pair) or bemcentinib (RH bar in each pair).

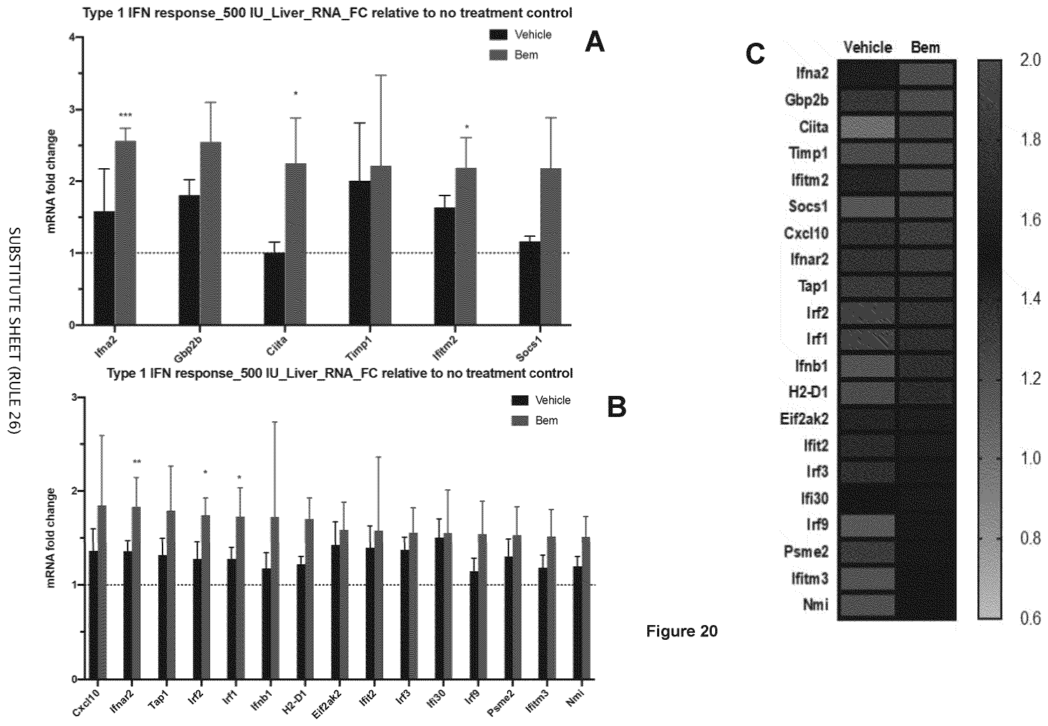

FIG. 20
Expression of IFN related genes in the liver at day 3 of infection with 500 iu MHV in vehicle control treated mice. A) Gene upregulated 2 fold by bemcentinib. B) Genes upregulated 1.5 fold by bemcentinib. Vehicle treatment compared to untreated (black bars) or bemcentinib treated compared to untreated (red bars). C) Heat map of gene changes. Statistical significant (p<0.05) is noted by asterisks.

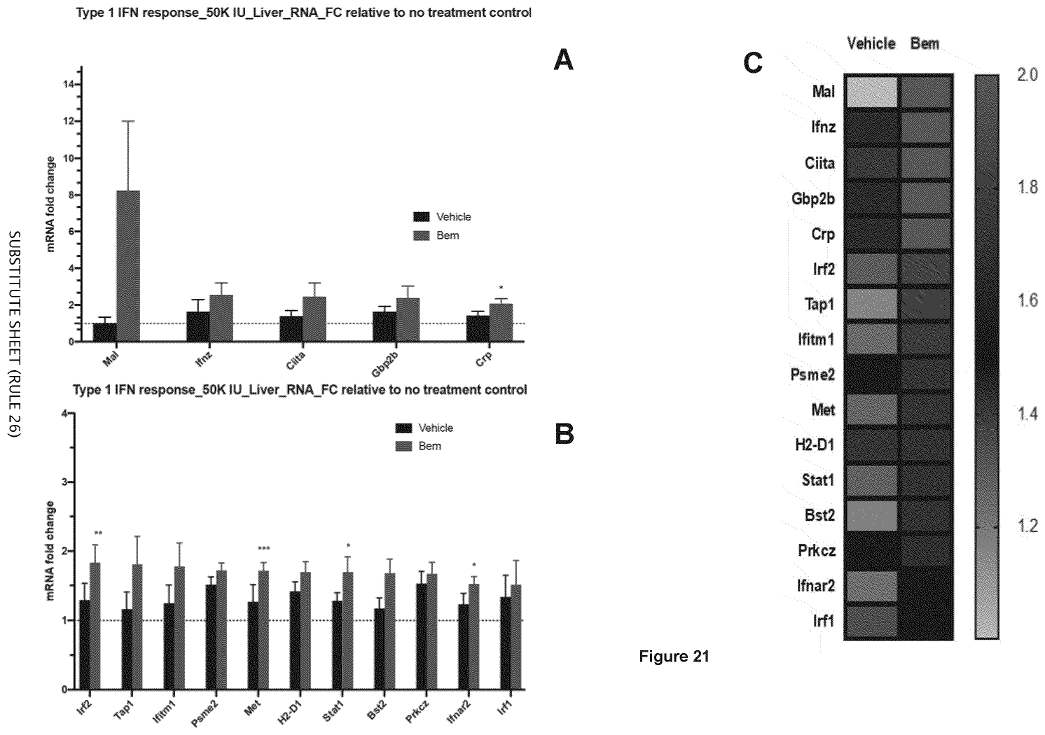

FIG. 21
Expression of IFN related genes in the liver at day 3 of infection with 50,000 iu MHV in vehicle control treated mice. A) Gene upregulated 2-fold by bemcentinib. B) Genes upregulated 1.5-fold by bemcentinib. Vehicle treatment compared to untreated (black bars) or bemcentinib treated compared to untreated (red bars). C) Heat map of gene changes. Statistical significant (p<0.05) is noted by asterisks.

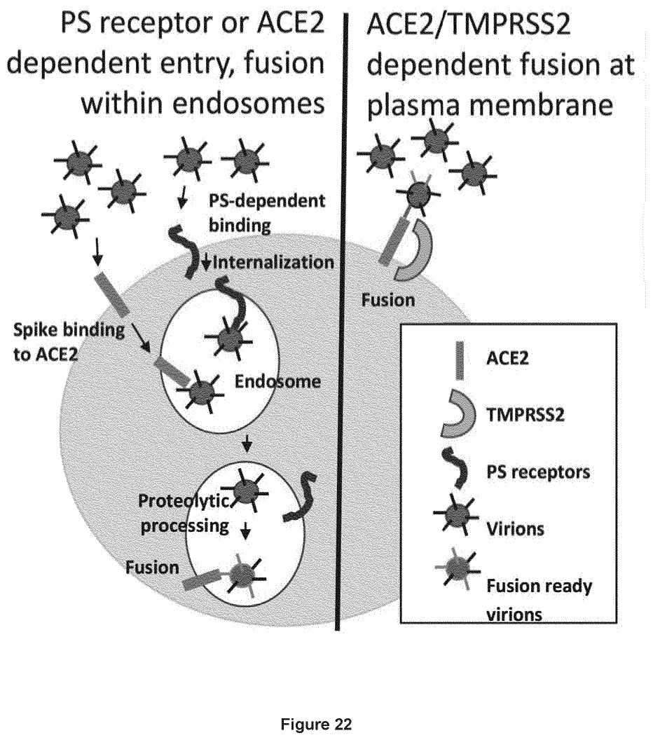

FIG. 22
A model of the dual routes of SARS-COV-2 entry.

FIG. 23
WHO COVID-19 9-point ordinal category scale (OCS) showing patient subsets receiving Bemcentinib (shaded area in column headed “BGBC020”)

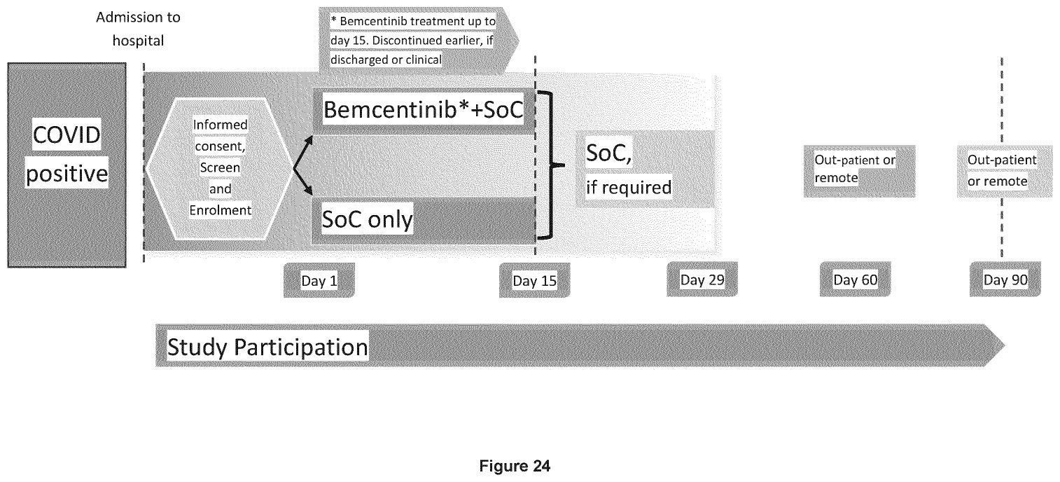

FIG. 24
BGBC020 & ACCORD-2 study schematic

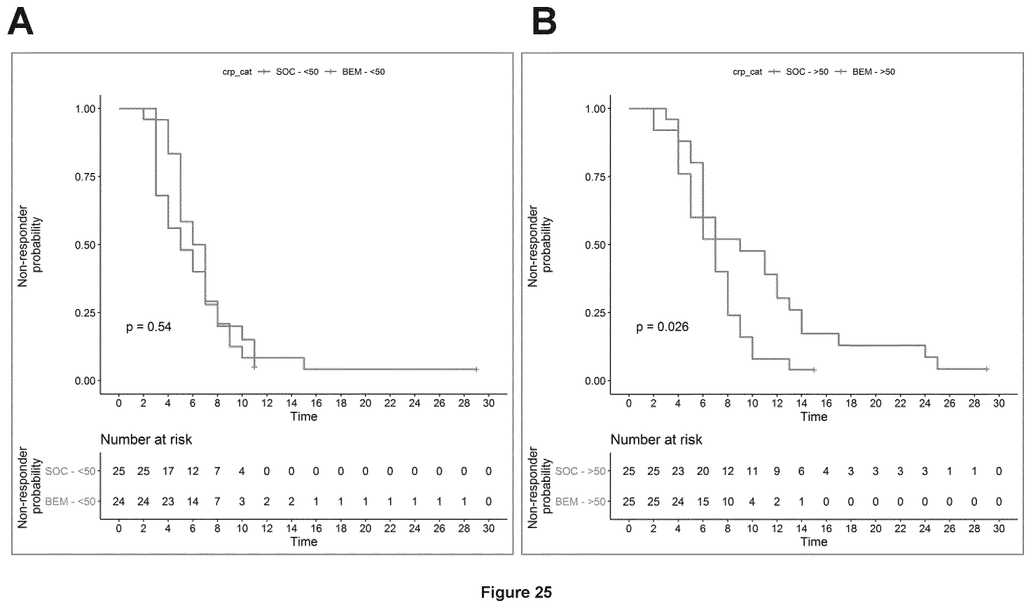

FIG. 25
BGBC020 interim results: Primary endpoint: stratified by baseline CRP −50 mg/L. Panel (A) is CRP <50 mg/L, Panel (B) is CRP ≥50 mg/L.

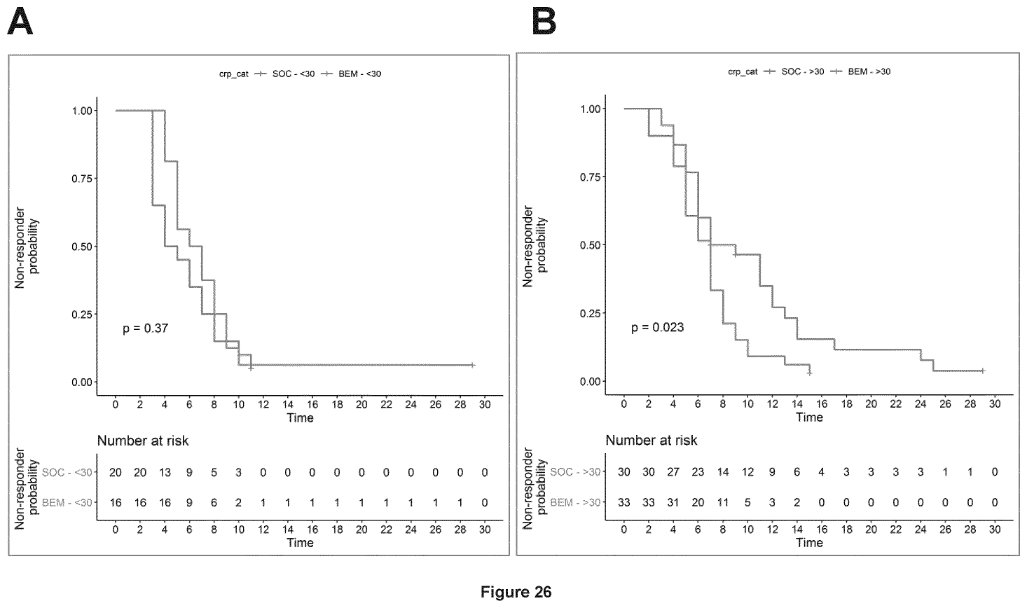

FIG. 26
BGBC020 interim results: Primary endpoint: stratified by baseline CRP −30 mg/L. Panel (A) is CRP <30 mg/L, Panel (B) is CRP ≥30 mg/L.

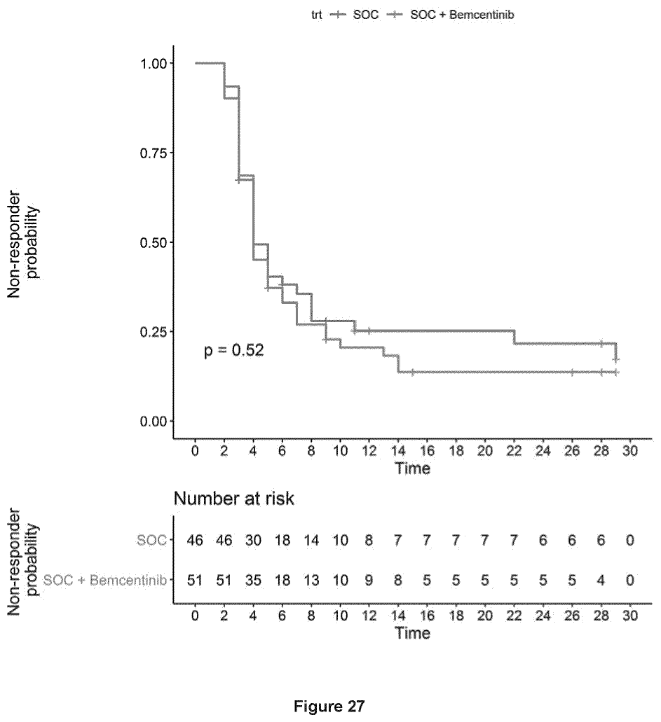

FIG. 27
BGBC020 interim results: Secondary Endpoint: Time to NEWS2 score ≤2. Hazard ratio (95% CI)=1.10 (0.70, 1.73)

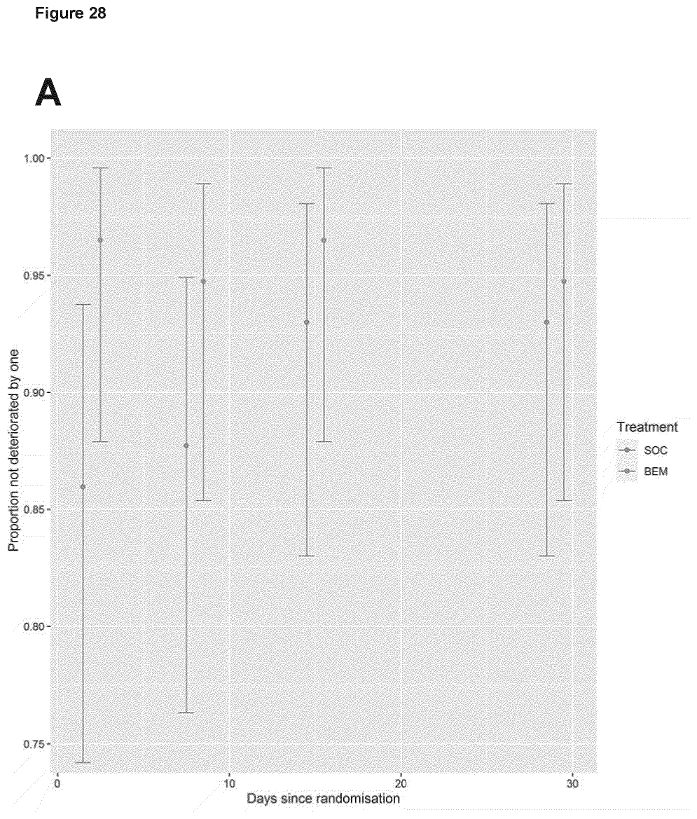

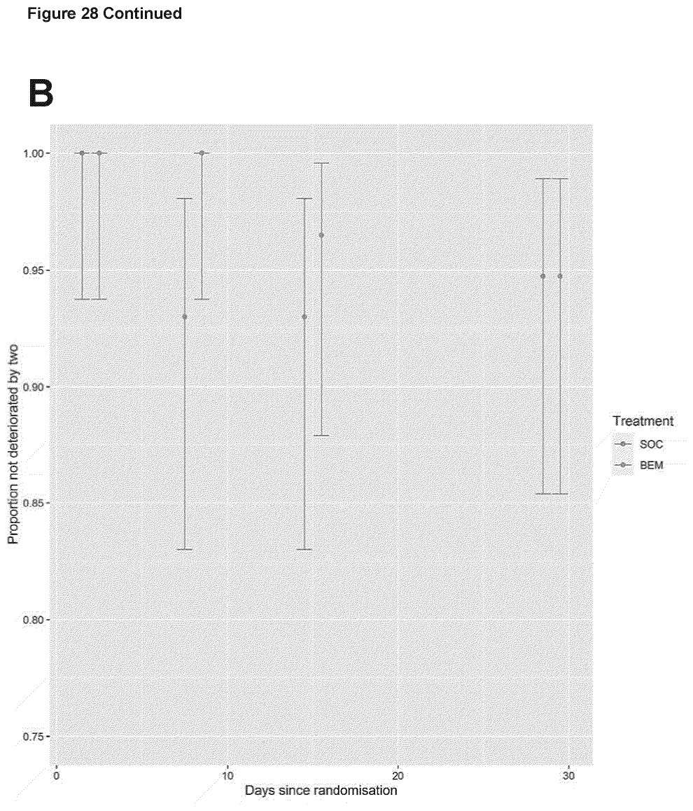

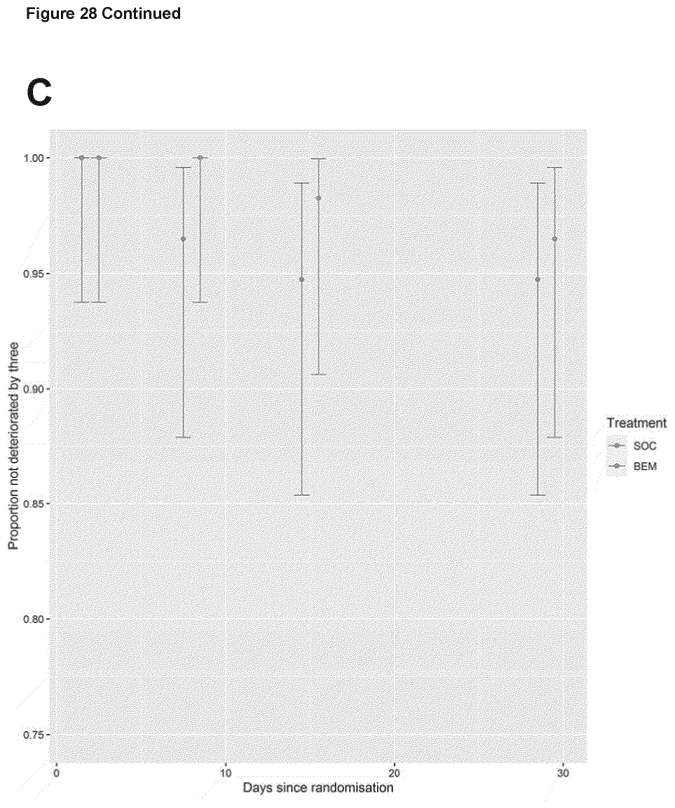

FIG. 28
BGBC020 interim results: Secondary Endpoint: avoidance of worsening by WHO scale at days 2, 8, 15 and 29. Panel (A)=worsening by 1 point on WHO scale, Panel (B)=worsening by 2 points on WHO scale, Panel (C)=worsening by 3 points on WHO scale. Throughout study period on-treatment, bemcentinib treatment associated with lower proportion of individual patients experiencing worsening, than standard of care alone

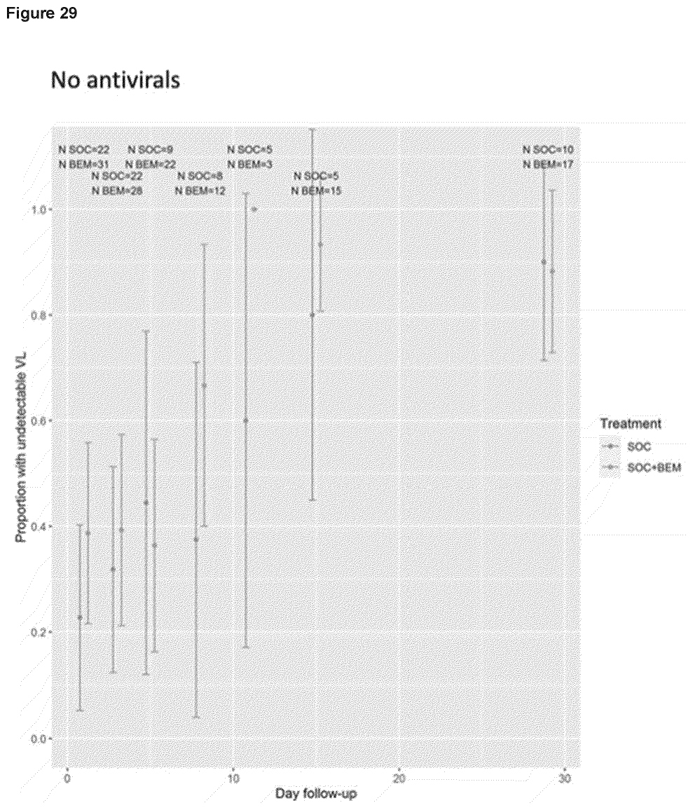

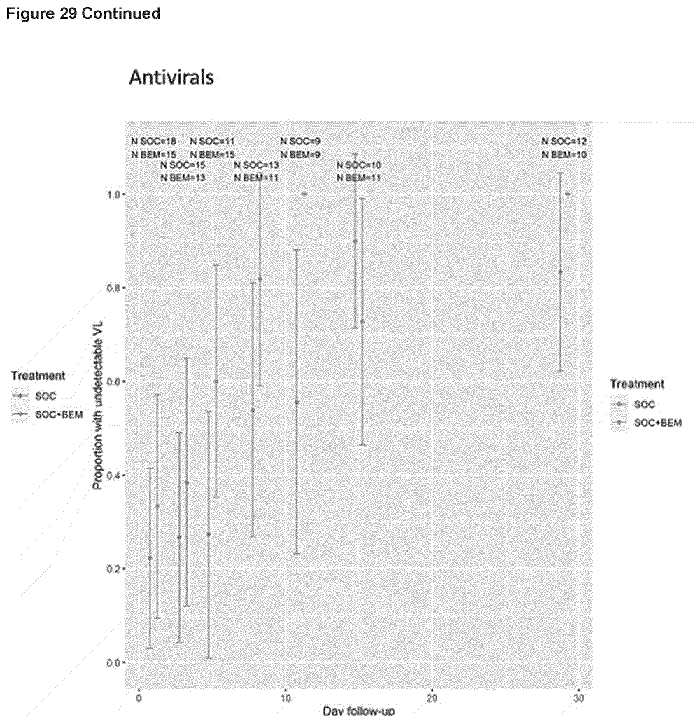

FIG. 29
Evaluation of SARS-CoV-2 salivary viral load in patients treated with bemcentinib in BGBC020

SEQUENCES
[10C9 Heavy CDR1]
SEQ ID NO. 1
DYNFTRYYIH
[10C9 Heavy CDR2]
SEQ ID NO. 2
WIYPGTGDSKYNEKFKG
[10C9 Heavy CDR3]
SEQ ID NO. 3
NGNYWYFDV
[10C9 Light CDR1]
SEQ ID NO. 4
RSSKSLLHSNGNTYLY
[10C9 Light CDR2]
SEQ ID NO. 5
RMSNLAS
[10C9 Light CDR3]
SEQ ID NO. 6
MQHREYPFT
[10G5 Heavy CDR1]
SEQ ID NO. 7
GYSFTDFYIN
[10G5 Heavy CDR2]
SEQ ID NO. 8
RIFPGGDNTYYNEKFKG
[10G5 Heavy CDR3]
SEQ ID NO. 9
RGLYYAMDY
[10G5 Light CDR1]
SEQ ID NO. 10
RSSQSLVHSNGIPYLH
[10G5 Light CDR2]
SEQ ID NO. 11
RVSNRFS
[10G5 Light CDR3]
SEQ ID NO. 12
SQGTHVPPT
[hu10G5 VH(GH1)]
SEQ ID NO. 13
EVQLVQSGAGLVQPGGSVRLSCAASGYSFTDFYINWvRQAPGKGLEWIAR
IFPGGDNTYYNEKFKGRFTLSADTSSSTAYLQLNSLRAEDTAVYYCARRG
LYYAMDYWGQGTLVTVSS
[hu10G5 VH(GH2)]
SEQ ID NO. 14
EVQLVESGGGLVQPGGSLRLSCAASGYSFTDFYINWvRQAPGKGLEWVAR
IFPGGDNTYYNEKFKGRFTLSADTSKSTAYLQMNSLRAEDTAVYYCARRG
LYYAMDYWGQGTLVTVSS
[hu10G5 VL(GL1)]
SEQ ID NO. 15
DIQMTQSPSSLSASVGDRVTITCRSSQSLVHSNGIPYLHWYQQKPGKAPK
LLIYRVSNRFSGVPSRFSGSGSGTDFTLTISSLQPEDFATYYCSQGTHVP
PTFGQGTKVEIK
[hu10G5 VL(GL2)]
SEQ ID NO. 16
DIQMTQSPSSLSASVGDRVTITCRSSQSLVHSNGIPYLHWYQQKPGKAPK
LLIYRVSNRFSGVPSRFSGSRSGTDFTLTISSLQPEDFATYYCSQGTHVP
PTFGQGTKVEIK
[10G5 GH1 Heavy chain]
SEQ ID NO. 17
EVQLVQSGAGLVQPGGSVRLSCAASGYSFTDFYINWVRQAPGKGLEWIAR
IFPGGDNTYYNEKFKGRFTLSADTSSSTAYLQLNSLRAEDTAVYYCARRG
LYYAMDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
[10G5 GH2 Heavy chain]
SEQ ID NO. 18
EVQLVESGGGLVQPGGSLRLSCAASGYSFTDFYINWVRQAPGKGLEWVAR
IFPGGDNTYYNEKFKGRFTLSADTSKSTAYLQMNSLRAEDTAVYYCARRG
LYYAMDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
[10G5 GL1 Light chain]
SEQ ID NO. 19
DIQMTQSPSSLSASVGDRVTITCRSSQSLVHSNGIPYLHWYQQKPGKAPK
LLIYRVSNRFSGVPSRFSGSGSGTDFTLTISSLQPEDFATYYCSQGTHVP
PTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
[10G5 GL2 Light chain]
SEQ ID NO. 20
DIQMTQSPSSLSASVGDRVTITCRSSQSLVHSNGIPYLHWYQQKPGKAPK
LLIYRVSNRFSGVPSRFSGSRSGTDFTLTISSLQPEDFATYYCSQGTHVP
PTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

### STATEMENTS OF INVENTION

1. A method for treating a virus infection in a subject, the method comprising administering to the subject an effective amount of an inhibitor of AXL activity or expression (AXLi).

2. A method for preventing or reducing transmission of a virus infection, the method comprising administering to the subject an effective amount of an inhibitor of AXL activity or expression (AXLi).

3. A method for increasing viral clearance from a subject, the method comprising administering to the subject an effective amount of an inhibitor of AXL activity or expression (AXLi).

4. The method according to any one of statements 1 to 3, wherein the virus infection is a coronavirus infection.

5. The method according to any one of statements 1 to 4, wherein the virus infection is an alphaletovirus infection. 6 The method according to any one of statements 1 to 4 wherein the virus infection is an orthocoronavirus infection.

7. The method according to any one of statements 1 to 4, or 6, wherein the virus infection is an alphacoronavirus infection.

8. The method according to any one of statements 1 to 4, or 6, wherein the virus infection is a betacoronavirus infection.

9. The method according to any one of statements 1 to 4, or 6, wherein the virus infection is a gammacoronavirus infection.

10. The method according to any one of statements 1 to 4, or 6, wherein the virus infection is a deltacoronavirus infection.

11. The method according to any one of statements 1 to 4, 6, or 8, wherein the virus infection is a betacoronavirus, lineage B, infection.

12. The method according to any one of statements 1 to 4, 6, 8, or 11, wherein the virus infection is a SARS-CoV infection.

13. The method according to any one of statements 1 to 4, 6, 8, or 11, wherein the virus infection is a SARS-CoV-2 infection.

14. The method according to statement 13, wherein the SARS-CoV-2 infection is caused by a variant comprising a mutation at position E484, optionally wherein the mutation is a E484K substitution.

15. The method according to either one of statements 13 or 14, wherein the SARS-CoV-2 infection is caused by a variant comprising a mutation at position N501, optionally wherein the mutation is a N501Y substitution.

16 The method according to any one of statements 13 to 15, wherein the SARS-CoV-2 infection is caused by a variant comprising a mutation at position K417, optionally wherein the mutation is a K417N or a K417T substitution.

17. The method according to any one of statements 13 to 16, wherein the SARS-CoV-2 infection is caused by a variant comprising a mutation at one or more of the positions L18, L242-244, D80, D215, D614, and A701, optionally wherein the variant comprises a mutation at each of the positions.

18. The method according to any one of statements 13 to 16, wherein the SARS-CoV-2 infection is caused by a variant comprising one or more of the mutations L18F, ΔL242-244, D80A, D215G, D614G, and A701V, optionally wherein the variant comprises all of the mutations.

19. The method according to any one of statements 13 to 18, wherein the SARS-CoV-2 infection is caused by the B.1.351 variant.

20. The method according to statement 13, wherein the SARS-CoV-2 infection is caused by the B.1.1.7 variant.

21. The method according to statement 13, wherein the SARS-CoV-2 infection is caused by the P.1 variant.

22. The method according to statement 13, wherein the SARS-CoV-2 infection is caused by the B.1.526 variant.

23. The method according to any one of statements 1 to 4, 6, or 8, wherein the virus infection is a betacoronavirus, lineage C, infection.

24. The method according to any one of statements 1 to 4, 6, 8, or 23, wherein the virus infection is a MERS-CoV infection.

25. The method according to any one of statements 1 to 24, wherein the AXLi is administered in combination with a second antiviral agent.

26. The method according to statement 25, wherein the second antiviral agent is selected from the group consisting of: a protease inhibitor, a helicase inhibitor, and a cell entry inhibitor.

27. The method according to either one of statements 25 or 26, wherein the second antiviral agent is selected from the group consisting of: ribavirin, an interferon, or a combination of both.

28. The method according to statement 25, wherein the second antiviral agent is remdesivir.

29. The method according to any one of statements 1 to 28, wherein the AXLi is administered in combination with an anti-inflammatory agent.

30. The method according to statement 29, wherein the anti-inflammatory agent is a corticosteroid.

31. The method according to statement 29, wherein the anti-inflammatory agent is a glucocorticoid steroid.

32. The method according to statement 29, wherein the anti-inflammatory agent is dexamethasone.

33. The method according to any one of statements 1 to 32, wherein the AXLi is administered in combination with an immunosuppressive agent.

34. The method according to statement 33, wherein the immunosuppressive agent is an IL-6 anatgonist.

35. The method according to statement 33, wherein the immunosuppressive agent is Tocilizumab.

36. The method of any one of statements 1 to 35, wherein the AXLi and/or second antiviral agent is comprised in a pharmaceutical composition, optionally further comprising a pharmaceutically acceptable excipient.

37. The method according to any one of statement 1 to 36, wherein the AXLi and/or the second antiviral agent is administered by inhalation.

38. The method according to any previous statement, wherein the subject is human.

39. The method according to any previous statement, wherein the subject has, is suspected of having, or is at high risk of having a viral infection.

40. The method of any preceding claim wherein the subject is a healthcare professional.

41. The method according to any one of statements 1 to 40, wherein the subject is at risk of severe symptoms if they were to catch the viral infection.

42. The method according to any one of statements 1 to 41, wherein the subject has one or more comorbidity selected from: respiratory system disease, cardiovascular disease, diabetes, hypertension, cancer, or a suppressed immune system.

43. The method according to statement 42, wherein the subject has two or more comorbidities.

44. The method according to statement 43, wherein the subject has three or more comorbidities.

45. The method according to any one of statements 1 to 44, wherein the subject is at least 60 years old.

46. The method according to any one of statements 1 to 45, wherein the subject is at least 70 years old.

47. The method according to any one of statements 1 to 46, wherein the subject is at least 80 years old.

48. The method according to any one of statements 1 to 47, wherein the subject is male.

49. The method according to any one of statements 1 to 48, wherein the subject's CRP level is at least 30 μg/mL.

50. The method according to any one of statements 1 to 48, wherein the subject's CRP level is at least 50 μg/mL.

51. The method according to any one of statements 1 to 50, wherein the subject is selected for treatment on the basis of having one or more of the features of statements 39 to 50.

52. The method of any one of statements 1 to 51, wherein the AXLi is a compound of formula (I):

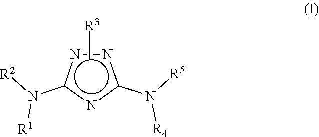

wherein:
R¹, R⁴ and R⁵ are each independently selected from the group consisting of hydrogen, alkyl, alkenyl, aryl, aralkyl, —C(O)R⁸, —C(O)N(R⁶)R⁷, and —C(═NR⁶)N(R⁶)R⁷;
R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR⁸, —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2);
or R² is a polycyclic heteroaryl containing more than 14 ring atoms as described above and R³ is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
or R³ is a polycyclic heteroaryl containing more than 14 ring atoms as described above, and R² is selected from the group consisting of aryl and heteroaryl, where the aryl and the heteroaryl are each independently optionally substituted by one or more substitutents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
each R⁶ and R⁷ is independently selected from the group consisting of hydrogen, alkyl, alkenyl, alkynyl, haloalkyl, haloalkenyl, haloalkynyl, hydroxyalkyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, haloalkenyl, haloalkynyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, and optionally substituted heteroarylalkynyl;
each R⁹ is independently selected from the group consisting of a direct bond, an optionally substituted straight or branched alkylene chain, an optionally substituted straight or branched alkenylene chain and an optionally substituted straight or branched alkynylene chain;
each R¹⁰ is independently selected from the group consisting of an optionally substituted straight or branched alkylene chain, an optionally substituted straight or branched alkenylene chain and an optionally substituted straight or branched alkynylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or two R^{12's}, together with the common nitrogen to which they are both attached, form an optionally substituted N-heterocyclyl or an optionally substituted N-heteroaryl;
each R¹³ is independently selected from the group consisting of a direct bond, an optionally substituted straight or branched alkylene chain and an optionally substituted straight or branched alkenylene chain; and
each R¹⁴ is independently selected from the group consisting of an optionally substituted straight or branched alkylene chain and an optionally substituted straight or branched alkenylene chain;
as an isolated stereoisomer or mixture thereof or as a tautomer or mixture thereof, or a pharmaceutically acceptable salt or N-oxide thereof.

64. The method of any one of paragraphs 1 to 62, wherein the AXLi is selected from the group consisting of:
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N3-(7-(S)-pyrrolidin-1-yl-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N³-((7S)-7-(2-butylamino)-6,7,8,9-tetrahydro-5H-benzo[7]annulene-2-yl)-1H-1,2,4-triazole-3,5-diamine;
or pharmaceutically acceptable salts thereof.

53. The method of any one of statements 1 to 51, wherein the AXLi is 1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine, or a pharmaceutically acceptable salt thereof.

54. The method of any one of statements 1 to 51, wherein the AXLi is bemcentinib.

55. The method of any one of statements 1 to 51, wherein the AXLi is selected from the group consisting of:
Dubermatinib (CAS No. 1341200-45-0; UNII 14D65TV20J);
Gilteritinib (CAS No. 1254053-43-4; UNII 66D92MGC8M);
Cabozantinib (CAS No. 849217-68-1; UNII 1C39JW444G);
SGI7079 (CAS No. 1239875-86-5);
Merestinib (CAS No. 1206799-15-6; UNII 5OGS5K699E);
Amuvatinib (CAS No. 850879-09-3; UNII SO9S6QZB4R);
Bosutinib (CAS No. 380843-75-4; UNII 5018V4AEZ0);
Sitravatinib (CAS No. 1123837-84-2; UNII CWG62Q1VTB);
XL092;
Glesatinib (CAS No. 936694-12-1; UNII 7Q290XD98N); and
foretinib (CAS No. 849217-64-7; UNII 81FH7VK1C4).

56. The method of any one of statements 1 to 51, wherein the AXLi is an antibody.

57. The method of statement 56 wherein the antibody is selected from the group consisting of:
the 1613F12 antibody disclosed in WO/2013/064685;
the 110D7 antibody disclosed in WO/2014/068139;
the 1003A2 antibody disclosed in WO/2014/068139;
the 1024G11 antibody disclosed in WO/2014/068139;
the hu10G5 antibody disclosed in WO/2017/220695; and
the YW327.6S2 antibody disclosed in WO/2011/159980.

58. The method of statement 56, wherein the antibody comprises the 6 CDRs having the sequences of SEQ ID Nos. 1 to 6.

59. The method of statement 56, wherein the antibody comprises the 6 CDRs having the sequences of SEQ ID Nos. 7 to 12.

60. The method of statement 56, wherein the antibody comprises:
a VH domain having the sequence of SEQ ID No. 13 and a VL domain having the sequence of SEQ ID NO.15;
a VH domain having the sequence of SEQ ID No. 13 and a VL domain having the sequence of SEQ ID NO.16;
a VH domain having the sequence of SEQ ID No. 14 and a VL domain having the sequence of SEQ ID NO.15; or
a VH domain having the sequence of SEQ ID No. 14 and a VL domain having the sequence of SEQ ID NO.16.

61. The method of statement 56, wherein the antibody comprises all 6 of the CDRs comprised in:
a VH domain having the sequence of SEQ ID No. 13 and a VL domain having the sequence of SEQ ID NO.15;
a VH domain having the sequence of SEQ ID No. 13 and a VL domain having the sequence of SEQ ID NO.16;
a VH domain having the sequence of SEQ ID No. 14 and a VL domain having the sequence of SEQ ID NO.15; or
a VH domain having the sequence of SEQ ID No. 14 and a VL domain having the sequence of SEQ ID NO.16.

62. The method of statement 56, wherein the antibody is Tilvestamab,

63. A pharmaceutical composition comprising an AXLi according to any one of statements 52 to 62 and a second anti-viral agent.

64. The pharmaceutical composition of statement 5, wherein the second anti-viral agent is as defined in any one of statements 25 to 35.

65. An AXLi according to any one of statements 52 to 62, a composition comprising an AXLi according to any one of statements 52 to 62, or the composition of either one of statements 63 or 64, for use in a method of treatment according to any one of statements 1 to 51.

66. Use of an AXLi according to any one of statements 52 to 62, a composition comprising an anti-proliferative agent according to any one of statements 52 to 62, or the composition of either one of statements 63 or 64, in the manufacture of a medicament for treating a disorder in a subject, wherein the treatment comprises the method of any one of statements 1 to 51.

### EXAMPLES

### Example 1: Bemcemtinib Inhibits Mouse Hepatitis Virus Infection of Murine BMDMs

### Drugs

A 1 μM solution of Bemcentinib (BGB324/R428, BerGenBio ASA, Bergen, Norway; referred to in figures as “BGB”) was prepared in DMSO for in vitro studies.

### Viruses

Mouse Hepatitis Virus (MHV) strain A59.

MHV is an enveloped RNA virus of the family Coronaviridae. It is common in both wild and laboratory mice, transmissible through aerosols, fomites, and direct contact. The virus is highly contagious, although not persistent in the environment.

Intranasal inoculation of sublethal doses of murine MHV-A59, a hepatic and neuronal tropic coronavirus, has been reported to induce acute pneumonia and severe lung injuries in C57BL/6 mice. Inflammatory leukocyte infiltrations, hemorrhages and fibrosis of alveolar walls can be observed 2-11 days after MHV-A59 infection. This pathological manifestation is associated with dramaticelevation of tissue IP-10 and IFN-γ and moderate increase of TNF-α and IL-1β, but inability of anti-viral type I interferon response. Accordingly, MHV-A59 has been proposed as a surrogate mouse model of acute respiratory distress syndrome by SARS-CoV and MERS-CoV infections. [See Yang, Z., et al., 2014, Virol. Sin. 29, 393-402; https://doi.org/10.1007/s12250-014-3530-y.]

### Cells

Experiments were performed either in wild-type (WT) cell or ISG15 knock-out cells (ISG15 KO). ISG15 is an interferon (IFN)-a/b-induced ubiquitin-like protein with a demonstrated role in murine antiviral immunity; accordingly, ISG15-deficient murine cells have enhanced susceptibility to viral infection (see Speer et al. 2015, Nat. Comms. 7:11496; doi:10.1038/ncomms11496).

### Protocol

Bone marrow derived macrophages (BMDMs) were isolated and matured for 6 days and plated in a 48 well format.

MHV (A59) was serially diluted onto cells in the absence (n=3 wells) or presence (n=2 wells) of 1 μM Bemcemtinib (BGB).

Cells were washed at 20 of infection with PBS and lyzed with TRIzol. RNA was isolated, reverse transcribed and amplified for the murine housekeeping gene Hypoxanthine guanine Phosphoribosyl Transferase (HPRT; see Silver et al. 2008, BMC Mol Biol. 9: 64) or an MHV gene.

### Results

Results are shown in FIG. 2A (WT cells) and FIG. 2B (ISG15 KO cells).

Columns shown are summary means of virus load (CT) relative to HPRT present in cells following the addition of 0.1 ul or 0.01 ul or 0.001 ul of virus solution (0.001 ul tested for ISG15 samples only). Each viral dose was tested in the presence (“BGB” columns) or absence (“WT” or “KO” columns) of 1 μM Bemcemtinib

BGB treated samples were tested in duplicate (n=2).

### Example 2: Preliminary Clinical Report of Bemcemtinib Efficacy Versus COVID-19

### Subject Details & Medical History

67 year old female patient with Stage IV EGFR+ (Exon 20 mutation) non small cell lung cancer. Ex-smoker.

### Comorbidities:

Type II diabetes (2 drugs: metformin, glipizide)
Hypertension (4 drugs: Losartan, hydrochlorothiazide, diltiazem, metoprolol)
Hyperlipidemia (2 drugs: rosuvastatin)
Asthma (2 drugs: albuterol, breo-ellipta)
Ischaemic heart disease (3 drugs: nitroglycerin, aspirin, metoprolol)
[several other comorbidities]

Prior partial response to erlotinib monotherapy.

Commenced bemcentinib+erlotinib combination treatment on 28th August 2018. Treatment ongoing at time of COVID-19 infection (cycle 28 commenced 18^{th} March 2020). Currently stable disease.

### COVID-19 Infection & Symptom History

### Day 1 (Thursday March 26th 2020)

subject reports feeling slightly unwell but no symptoms significant enough to report separately unwell enough to report anything of note
   Day 3 to 4 (Saturday 28^{th}/Sunday 29^{th} March)
ongoing fever of 101 F (38.3 C)
one episode of mild cough, no dyspnea (pulse oximetry 96%-97% on air; repeatedly self testing at home)
myalgia, headache, and anosmia
persistent nausea
one episode of diarrhea

### Day 5 (Monday 30^{th} March)

attended local hospital; throat swab taken for Sars-CoV-2 testing
milder fever reported, though normal (36.9 C/98.4 F) on examination post paracetamol at the weekend and O/E Temp 36.9 C/98.4 F
persistent nausea

### Day 7 (Wednesday 1^{st} April)

swab test confirmed subject as COVID-19 positive
Subject reported improving condition, with no fever, dyspnea, or cough
nausea settling

### Day 9 (Friday 3^{rd} April 2020)

Subject reports feeling well
co-habiting with husband who has shown no signs of COVID-19 infection

### Clinical Observations

Subject's course of COVID-19 considered very mild by subject and supervising clinician, with only slight episode of cough and no other respiratory symptoms.

Other recognized COVID-19 symptoms (fever, anosmia, myalgia, headache) also considered mild by patient and clinician.

Mild COVID-19 symptoms and attenuated (7 day) disease course considered remarkable in this high-risk subject with multiple comorbidities known to significantly increase risk of serious COVID-19 disease. Apparent lack of transmission to co-habiting partner also of note.

Recommendation for further investigation of COVID-19 infections in subjects receiving bemcentinib, including matched controls.

### Example 3: Near-Term Preclinical and Clinical Research Plan

### Preclinical

Further in vitro analysis of the effect of MHV infection on mBMDM including MOI dilutions and bemcentinib dose-response
Evaluate the effect of bemcentinib on SARS-CoV-2 infection of Vero cell lines including both wild type, Axl knockout, and PS-receptor TIM-1 knockout (BSL3-level experiments)
Evaluate the effect of bemcentinib on SARS-CoV-2 infection of mBMDM (human ACE-2 negative) and hACE-2+ mBMDM, including MOI dilutions and bemcentinib dose-response
Evaluate the effect of bemcentinib on SARS-CoV-2 infection of primary human airway epithelial cells (BSL3-level experiments)
Evaluate potential interactions (for synergy) with ACE2r blockers.
Evaluate bemcentinib treatment on MHV-infected mice (protocol as per previous EBOV in vivo studies).

### Clinical

A) Longitudinal Case Control Real World Evaluation of Patients Currently on Bemcentinib This is an ideal real world analysis of prophylaxis of an at risk cohort of patients allowing evaluation of early type I interferon response to attenuate the early phase of coronavirus infection. The total cohort size is 110 patients: 55 cancer patients on Bemcentinib treatment v 55 matched controls:
   1) Sequential weekly evaluation of:
      Nasal and pharyngeal swab for SARS-CoV-2 RTPCR assay
      IgM, IgG and IgA analysis of SARS-CoV-2 specific immune response
      Hematology analysis to identify lymphocytopenia, thrombocytopenia and leucopenia
      Biochemistry analysis to identify alteration of: CRP, D-dimer, creatine kinase, lactate dehydrogenase, liver function tests, renal biochemistry and electrolytes, ferritin assay, IL6
      Cytokine profiling weekly and more intensively in those developing features of early infection
      Sequential serum and plasma samples for additional biomarker studies
   2) Clinical Evaluation
      1⁰ composite endpoint: Rate of Admission and ICU Admission, Rate of mechanical ventilation, Rate of death, days in hospital
      Weekly temperature and regular recording by patient
      Evaluation of cough cf. COVID-19 symptom score as per ongoing clinical trials
      Documentation of comorbidities in addition to cancer e.g. Cancer type, Diabetes, Hypertension, COPD, Arthritis, CKD, NASH
      Documentation of concomitant medications—statins, antihypertensives (ACE inhibitors), antinflammatories, QT liability drugs, metformin, insulin, statin, empafliglozin, NSAIDS and aspirin, inhaled steroids
B) Clinical trial preliminary proposal
   Option 1: Access one of the national or international COVID-19 trial platforms such as the Adaptive COVID-19 Treatment Trial NCT04280705
   Option 2: Through partnership design and conduct a randomised trial of bemcentinib v SOC (placebo would take time to produce) in at risk populations (see below), and mild to moderate COVID 19 infection (each trial N approx. =400), the aim being to induce and early type I interferon response to attenuate the disease. Randomization would be stratified by: 1) site and 2) severity of illness at enrolment:
      The primary objective of the study is to evaluate the clinical efficacy of bemcentinib vs SOC in patients hospitalized with COVID-19 in attenuating the infection in those at risk and those developing early infection
      Target populations include those at risk (patients >70, or patients with comorbidity risks currently leading to a recommendation of self-isolation (eg diabetes, hypertension, COPD, CHD and others)
      mild-moderate disease (SpO2 >94% and respiratory rate <24 breaths/min without supplemental oxygen)
      Subjects will be assessed daily while hospitalized. Discharged patients will be asked to attend study visits at Days 15, and 29.
      All subjects will undergo a series of efficacy, safety, and laboratory assessments.

### Example 4: Further Analysis of the Effect of Bemcentinib on Mouse Betacoronavirus (Mouse Hepatitis Virus, MHV) Infection of Mouse Bone Marrow-Derived Macrophages (mBMDM) In Vitro

In addition to the experiments described in Example 1, the below further analysis was conducted.

Bone marrow derived macrophages (mBMDM) were isolated, matured and infected with MHV in the presence of increasing concentrations of bemcentinib for 24 h (see FIG. 3A). Increasing doses of bemcentinib demonstrated an inhibitory trend with 1 μM of drug significantly reducing infection (p=0.034 in one-way ANOVA). As mBMDM are primary cells that have intact innate immune responses, protection conferred by bemcentinib may be due to either reduced virus entry into these cells or enhanced innate immune responses.

A second group of MHV studies was performed with matured mBMDM from WT, IGS15 knock-out (KO) or USP18^{C61A/C61A} knock-in mice. ISG15 is a type I interferon stimulated gene encoding a ubiquitin-like protein. ISGylation of both viral and host proteins contributes to control of a wide variety of virus infections. The UBC18 ISG-deconjugase removes ISG-groups from proteins; the USP18(C61A) mutant lacks enzyme activity and USP18^{C61A/C61A} knock-in mice have elevated levels of ISGylation upon initiation of ISG15 activity since the ISGylation eraser function is not available.

The effect of bemcentinib on MHV replication in mBMDM from these three different mouse strains was assessed. As shown in FIG. 3B, ISG15 expression and function impacted MHV infection. The loss of ISG15 (KO) resulted in a higher virus load that did not reach statistical significance. In contrast, the USP18^{61A/C61A} mutant had statistically lower virus load than WT cells. Treatment with bemcentinib (1 μM) significantly reduced MHV infection WT cells. While, bemcentinib reduction of virus load in mBMDM from ISG15 (KO) and USP18^{C61A/C61A} strains did not reach statistical significance, virus load consistently trended lower in the presence of bemcentinib in all 3 lines, suggesting that bemcentinib inhibition of MHV in mBMDM is not primarily due to ISG15 expression. These results are consistent with a role for AXL in both viral uptake and an interferon-response suppression.

### Example 5: Effect of Bemcentinib on SARS-CoV-2 Infection In Vitro of Vero Cell Lines Including Both Wild-Type, AXL Knockout and PS-Receptor TIM-1 Knockout

SARS-CoV-2 (2020 WA_1) was obtained from the Centers for Disease Control (Atlanta) for BSL3-level studies to establish a role for AXL in SARS-CoV-2 infection.

### HEK 293T Cell Findings

These cells were used to determine the prerequisites of SARS-CoV-2 infection. There is no detectable SARS-CoV-2 infection in WT HEK 293T cells, thus allowing different combinations of putative viral receptors to be exogenously expressed. This provides an opportunity to determine the role of putative viral receptors in SARS-CoV-2 infection. As shown in FIG. 4A, entry of VSV pseudovirions that express luciferase and are pseudotyped with SARS-CoV-2 spike protein, is enhanced by hACE2, consistent with the known role for this receptor. In these studies, low (suboptimal) levels of hACE2 plasmid were transfected, providing a suboptimal amount of hACE2 expression on the cell's surface and only a 7-fold increase in infection over baseline. Neither AXL nor TIM-1 expression supported significant SARS-CoV-2 spike protein-mediated viral uptake by themselves. However, the combined expression of low levels of hACE2 and the PS receptors, AXL or TIM-1, synergized, strongly elevating virus infection levels. Similar trends were observed in these transfected HEK293 cells in BSL3 studies with SARS-CoV-2 virus (FIG. 5). In HEK 293T cell studies, AXL consistently enhanced SARS-CoV-2 spike dependent entry, but the synergy was also consistently more modest than that observed with TIM-1 for reasons that are not currently clear. None of these receptors affected infection with VSV particles pseudotyped with Lassa virus glycoprotein (FIG. 4B). Similar studies were also performed with two other PS receptors, TIM-4 and TYRO3 (FIG. 4C). While TIM-4 synergized with hACE2, TYRO3 did not synergize despite equivalent levels of surface expression of this PS receptors.

Studies were performed with exogenous expression of hACE2 and the cell surface, serine protease TMPRSS2. The combination of hACE2 and TMPRSS2 expression is reported to mediate SARS-CoV-2 entry at the plasma membrane. While low concentrations of TMPRSS2-expressing plasmid co-expressed with hACE2 enhanced SARS-CoV-2 spike dependent entry, this effect was diminished with increasing concentrations of TMPRSS2 transfected, indicating that TMPRSS2 expression at high concentrations is deleterious, potentially due to spike degradation by TMPRSS2 protease activity (FIG. 5).

Additional studies were performed in these cells to understand the entry pathway utilized by SARS-CoV-2 virions. This virus has recently been shown to enter through both a cell surface mechanism that requires TMPRSS2 protease processing of SARS-CoV-2 spike and through the endosomal compartment where cathepsin proteases perform the requisite spike processing. TMPRSS2 activity is blocked by the serine protease inhibitors camostat or nafamostat, whereas cathepsins, are blocked by the cysteine protease inhibitor E645. HEK293T cells transfected with the various relevant receptors were left untreated or treated with E64 or camostat prior to pseudovirion infection (FIG. 6). Infection of human ACE2 transfected cells demonstrated E64 effectively blocked infection. Unexpectedly, camostat also blocked infection, but to a lesser extent. These same trends were observed in virus infected cells transfected with hACE2 and either of the PS receptors. Since HEK 293T cells do not endogenously express TMPRSS2, the virus inhibition by camostat suggests that in this cell line other serine proteases that are inhibited by camostat contribute to SARS-CoV-2 entry, perhaps through independent processing of spike.

### Vero E6 Cell Findings

The Green African Monkey (AGM) kidney cell line, Vero, endogenously expresses ACE2, AXL and TIM-1. Further, this species of monkey is sufficiently related to humans that most human specific reagents cross react with AGM proteins. Thus, these cells can be used to understand the role of endogenous levels of these proteins in SARS-CoV-2 infection. AXL, TIM-1 and combined KO lines have been generated for evaluating virus infection. A derivative cell line, Vero E6, expresses significantly more ACE2 and TMPRSS2, and presumably as a consequence this line supports more robust SARS-CoV-2 infection (FIG. 7).

Initial studies assessed the ability of VSV-GFP/SARS-CoV-2 spike pseudovirions (FIG. 8A) or WT virus (FIGS. 8B and 8C) to infect Vero E6 or the lung cancer cell line CalU3 in the presence of increasing doses of bemcentinib, anti-AXL mAb tilvestamab (BGB149) or other entry inhibitors. Increasing concentrations of bemcentinib inhibited pseudovirus infection of Vero E6 cells (FIG. 8A). A similar dose response curve was observed with WT SARS-CoV-2 infection of Vero E6 cells. Increasing doses of phosphatidylserine (PS) liposomes, a dominant-interfering virion mimetic, yielded similar results, suggesting that SARS-CoV-2 infection of these cells is dependent on virion/PS receptor interactions. Bemcentinib did not inhibit SARS-CoV-2 infection of CalU3 cells (FIG. 8B). The cathepsin inhibitor E64 and, to a lesser degree, BGB149 blocked infection of Vero E6 cells. These data provide evidence that SARS-CoV-2 traffics through the endosomal compartment of Vero E6 cells. These studies also suggest that SARS-CoV-2 utilizes PS receptors (and specifically AXL signaling) in Vero E6, but not CaIU3 cells. Note that this is in contrast to a recent study (Dittmar et al) where bemcentinib was reported to inhibit SARS-CoV-2 infection in both cell types. This may in part reflect differences in the source of CaIU3 cell lines used.

In an additional set of studies with Vero E6 cells, it was evaluated whether bemcentinib or PS liposomes blocked VT SARS-CoV-2 binding. Using an MOI of 5, SARS-CoV-2 was incubated at 15° C. for 60 m with Vero E6 cells in the presence of bemcentinib or PS liposomes. Phosphatidylcholine (PC) liposomes were incubated with the cells as a control for PS-mediated viral competition; trypsin was added after virus binding to some cells as a negative control. Cells were placed in Trizol and virus binding was assessed by qRT-PCR detection of viral RNA genomes as compared to the level of a housekeeping gen mRNA, GAPDH. As shown in FIG. 9, bemcentinib had no effect on virus binding. This was expected since the drug does not block PS-GAS6-AXL binding, but inhibits GAS6-mediated AXL kinase activity and cargo internalization. PS liposomes modestly, but significantly reduced virus binding to cells. Similar studies in Vero E6 cells and other more relevant lung cells will be performed to assess virus internalization following virus binding. It is anticipated that if AXL is important for internalization of virions, bemcentinib will significantly reduce the number of internalized virions.

Vero E6 cells as well as A549-hACE2 cells were used for RNAseq studies. In the case of Vero E6 cells, the cells were treated with bemcentinib, PC or PS liposomes in the presence or absence of SARS-CoV-2 infection. In the A549-hACE2 studies, cells were treated with bemcentinib with and without infection. Infections were harvested at 24 h and RNA isolated, qualitatively and quantitated analyzed and sent for RNAseq. BerGenBio personnel are performing the analysis. Preliminary results show that bemcentinib treatment dramatically reduced SARS-CoV-2 viral transcription (FIG. 10).

E2 expression on different cell types was examined. In Vero E6 cells and some other lines, ACE2 surface expression was limited to a smalle.

### Example 6: Effect of Bemcentinib on SARS-CoV-2 Infection of mBMDM (hACE2 Negative) and hACE2+ mBMDM, and Human Airway Epithelial Cells In Vitro

BMDM and hMDM Studies

It was quickly apparent that mouse cells and cell lines have minimal relevance in SARS-CoV-2 studies, but as noted above, bemcentinib did inhibit MHV infection in these cells (FIG. 3A). Thus, i I found that hMDMs required the introduction of human ACE2 in order to support infection. hACE2 e-Clu

### HAE Studies

Human airway epithelial cells (HAE) were obtained from the Cystic Fibrosis Center. These cells a io hi bnti.

### Lung Cell Lines

A selection of lung tumor lines and immortalized bron. T t t

| | Sensitive to | AXLprotein | Level of SARS- |
|---|---|---|---|
| Lung line | Bemcentinib | expression | CoV-2 infection* |
| HCC2302 | Yes | N/D | High |
| H322 | Yes | + | Medium |
| 820 | Yes | +++ | Low to medium |
| HCC4207 | Mixed findings | + | Low |
| H1650 | Yes | +++ | High |
| HCC4256 | No | +++ | Medium |
| H1819 | No | No | High |
| H3255 | No | + | Medium |
| HCC1944 | Mixed findings | + | High |
| CalU3 | No | +++ | Low |
| HBEC 26 | N/D | N/D | Low |
| HSAEC13 | Mixed findings | +++ | Low |

*High = virus load compared to housekeeping gene >1
Medium = virus load compared to housekeeping gene >0.01 to 0.1
Low = virus load compared to housekeeping gene <0.01

Table 1 shows a summary from nine ACE2+ lung tumor lines and CalU3 cells. T w Itau) r.c w er

### Huh7

A final cell type tested was the human hepatocellular carcinoma line, Huh7. While of little relevance to respiratory infections, this is well established line that expresses both AXL and TIM-1. Inhibitors of these PS receptors were added to cells prior to SARS-CoV-2 infection (MOI=0.1) and virus load was assessed at 24 h. Bemcentinib inhibited virus infection in a dose dependent manner, providing evidence that AXL signaling contributes to SARS-CoV-2 infection—either by AXL serving as an uptake receptor or by AXL-mediated alteration of type I IFN responses. Interestingly ARD5, a MAB that blocks human TIM-1 interactions with PS 7,8 u)i's

### Example 7: Evaluation of Potential Synergy with ACE2r Blockers

HEK293T cell studies (eg, FIG. 9) suggested that PS/PS receptor interactions can contribute to SARS-CoV-2 entry into cells. It was therefore postulated that combinations of inhibitory agents against PS receptors and ACE2 might synergize. Initial studies evaluated the ability of commercially available anti-ACE2 polyclonal antisera (R&D Systems) to inhibit VSV/SARS-CoV-2 spike infection and it was found that even with high concentrations of the antisera (6 μg/ml) that were reported to inhibit SARS-CoV-2 infection significant inhibition of virus infection in Vero E6 cells was not observed (FIG. 15). Since at the time that was the only known reagent to inhibit ACE2-dependent infection, the synergy studies were not performed. Instead, investigation shifted to examining the correlation of bemcentinitib inhibition with other SARS-CoV-2 entry inhibitors, camostat (and nafamostat) or E64 as shown above.

### Example 8: Evaluation of Anti-Human AXL Blocking Antibody (Tilvestamab) on SARS-CoV-2 Infection of Selected Human Cell Cultures

As shown in FIG. 8C, tilvestamab (BGB149) was observed to modestly inhibit WT SARS-CoV-2 replication inpnuA.NoSi,

### Example 9: Evaluation of Bemcentinib Treatment on MHV-Infected Mice

Ini Bem Tween 80) was administered by gavage twice daily to one group of male C57BL/6 mice (n=5) starting at day −1. A second group received vehicle and a third group was not gavaged. MHV(A5dmozr virus-induced cell killing of individual wells at day 5 of infection. As shown in FIG. 17A, viral titers in livers of the mice infected with 500 iu were negligible in all treatments.

In mice given 50,000 iu, amted with 500 iu were indistinguishable (FIG. 17B). Virus loads were higher in the 50,000 iu iurC)esur).

Livfu Ireted with 500 iu our). Spleen RNA in the 50,000 iu siclFαβ transcription factor that controls MHC class II expression. These data provide evidence that bemcentinib is enhancing type I interferon responses and by doing so enh (.

A Model for the Role of Axl in SARS-CoV-2 Entry into Cells

The data inform a model for the role of AXL in SARS-CoV-2 entry. Without ishing to be bound by theory, ACE2 and TMPRSS2 are found on the surface of some lung epithelial cells and, on those cells, it is likely that SARS-CoV-2 enters cells through fusion with the plasma membrane (FIG. 22; right hand side). For those cells that do not express TMPRSS2, the virus bound to ACE2 must be endocytosed to late endosomes for spike protein processing to form the fusion ready confirmation required for viral/cell membrane fusion. However, it was observed that some permissive cells do not express ACE2 on their surface, but abundantly express it intracellularly. Under these conditions, ACE2 may be present in the endosomal compartment, but the virus must reach that compartment to be proteolytically processed and interact with ACE2. It is proposed that PS receptors serve as a route for the virus to reach the endosomal compartment (FIG. 22, left hand side). The evidence described herein indicates that PS receptors, TIM-1, TIM-4 and AXL, enhance virus infection in some cells. Specifically, AXL is expressed abundantly on many of the lung epithelial cell lines examined to date and it is proposed that AXL can serve as an alternative route of entry into some lung epithelial populations. In addition, viral engagement of AXL may suppress type I IFN responses. This is likely a second, independent mechanism that enhances SARS-CoV-2 infection.

### Example 10: Phase 2 Clincial Trial in India & South Africa (BGBC020) and the United Kingdom (ACCORD-2)—Interim Results

### BGBCO20 Overview

### Sponsor:

### BerGenBio ASA

### Countries:

### India—CTRI/2020/10/028602

### South Africa—DOH-27-092020-6170

### Enrolment:

### Oct 2020-March 2021

### Status:

India enrolment: 60 patients; 30 Bemcentinib, 30 Standard-of-Care [SoC]
South Africa enrolment: 55 patients; 28 Bemcentinib, 27 SoC]
Study enrolment complete at 96% of target (115), 4 Mar. 2021

| Baseline | Baseline Intent | | | |
|---|---|---|---|---|
| WHO OCS | to use steroid | Bemcentinib | SOC | Total |
| 3 | N/A | 6 | 5 | 11 |
| | No | 11 | 10 | 21 |
| 4 | Yes | 36 | 36 | 72 |
| 5 | No | 1 | 1 | 2 |
| | Yes | 4 | 5 | 9 |
| | Total | 58 | 57 | 115 |

Study schematic is shown in FIG. 24.

### ACCORD-2 Overview

### Sponsor:

University of Southampton (ACCORD2-NIHR phase 2 platform study)

### Country:

### UK (EudraCT 2020-001736-95)

### Enrolment:

Commenced May 2020; halted in summer 2020.
Restart after Substantial amendment (November 2020)

### Status:

Ongoing 25 Mar. 2021-50% of target recruitment (60 patients)

### Inclusion & Eligibility (all Studies)

Inclusion based on WHO COVID-19 9-point ordinal scale, with Bemcentinib administered to patients scoring 3, 4, or 5 (South African arm) and 4 or 5 (Indian arm)—see FIG. 23.

Inclusion criteria:
1) Adults SARS-CoV-2 infection confirmed
2) symptoms and/or signs consistent with COVID-19, requiring treatment
3) A score of Grade 3 to 5 on the 9-point ordinal scale (4 or 5 in India)
4) Agree to adhere to contraception and breastfeeding requirements during and post study
   Exclusion criteria:
1) Recovery from previous more severe disease (Grade 6 or 7)
2) Unable to swallow capsules
3) QTcF longer than 470 msec
4) Uncorrected low K+
5) Tarnsaminase >5× ULN
6) Stage 4 renal failure
7) Taking other experimental SARS-CoV-2 therapy
8) Known TB, HIV, hepatitis

### Endpoints (all Studies)

Primary endpoint:
Time to the earliest of:
   (a) clinical improvement of at least 2 points (from randomization) on WHO 9-point scale, and
   (b) live discharge from the hospital.
      Key secondary endpoints:
(1) The proportion of patients not deteriorating according to the ordinal scale by 1, 2, or 3 points on Days 2, 8, 15, 22, and 29;
(2) Duration (days) of oxygen use and oxygen-free days;
(3) To evaluate SARS-CoV-2) viral load
   a. qPCR determination of SARS-CoV-2 in oropharyngeal/nasal swab while hospitalised on Days 1, 3, 5, 8, 11, 15, and (optional) Day 29
      Other secondary endpoints:
To evaluate ventilator-free days and incidence and duration of any form of new ventilation
To evaluate duration of organ support (eg, including respiratory, renal, and cardiac support
To evaluate response rate (see primary endpoint for definition of responder)
To evaluate time to discharge
To evaluate overall mortality
Change in the ratio of the oxygen saturation to fraction of inspired oxygen concentration (SpO₂/FiO₂)
To evaluate intensive care unit (ICU) and hospitalisation length
To evaluate National Early Warning Score 2 (NEWS2)
To evaluate improvement taking into account worsening and death
   Exploratory endpoints:
Viral load: Quantitative PCR of SARS-CoV-2 in blood (on Day 1) and saliva (while hospitalised) on Days 1, 3, 5, 8, 11, 15, and 29
To collect samples for translational research on viral genomics and serum antibody production

### Interim Results

Summary of key findings:
(1) Improved survival
   a. Fewer COVID-19 related deaths in bemcentinib arm versus SoC (3 versus 10) across both BGBC020 and ACCORD-2 studies
   b. See ‘Survival & safety Data’ below
(2) Reduced time to WHO OCS improvement or discharge (only data for BGBC020 available as of filing date)
   a. Favours the bemcentinib arm, particularly in the group with higher baseline CRP (C-reactive protein; high levels are indicative of more severe infection)
   b. See FIGS. 25 & 26
(3) Reduced clinical degree of severity using NEWS2 (only data for BGBC020 available as of filing date)
   a. NEWS2 normalisation favours bemcentinib arm within week 1
   b. See FIG. 27
(4) Improved viral clearance (only data for BGBC020 available as of filing date)
   a. Higher rates of viral clearance in the bemcentinib arm (100% at day 11, versus 57% with SoC)
   b. See ‘Evaluation of salivary viral load’ below

### Survival & Safety Data

BGBC020
   Patients enrolled (115 in total)
      SoC—57 patients
      Bem—58 patients
   Mortality
      SoC—4 patients (3 by D29 plus 1 at D36)
      Bemcentinib—2 patients (by D29)
   SAE: SoC—4 patients; Bem—5 patients
      No SUSAR
   No QTcF prolongation >501 msec on bemcentinib
ACCORD-2
   Patients enrolled (60 in total)
      SoC—30 patients
      Bem—30 patients
   Mortality
      SoC—6 patients (by D29)
      Bem—1 patients (by D29)
   Laboratory (no TRAE haem or chemistry) of note
   No reported QTcF prolongation
      Evaluation of SARS-CoV-2 Salivary Viral Load in Patients Treated with Bemcentinib in BGBC020

Qualitative and/or quantitative PCR determination of SARS-CoV-2 in blood (on Day 1) and saliva (while hospitalised) on Days 1, 3, 5, 8, 11, 15, and 29

The number and percentage of patients with a positive and negative result for SARS-CoV-2 based on oropharyngeal/nasal swab, blood and saliva will be provided by treatment arm and visit based on the Intention to Treat Analysis Set for PCR test results only.

### Methods and Material

Saliva for SARS CoV 2 PCR (qualitative and quantitative)

Collection, processing and shipping: Patients were instructed not to eat or drink for 30 minutes prior to saliva sample collection. On instruction, the patient was asked to not to swallow saliva and as it gathered in the mouth to spit instead into a 10 mL Plain Cap Sarstedt tube, until 5 mL was collected (minimum 3 mL required for sample).

Once sample collected an equal volume of the provided RNA later solution was added to the collection tube from the vial provided and the sample was mixed by flicking the tube before freezing for transport to Q² Solutions, Edinburgh, UK.

### Analytic Methods

The TaqPath COVID-19 Combo Kit has been approved by the US FDA under Emergency Use Authorization (EUA), 1 and has been verified at the Q2 Solutions RTP and Valencia laboratories.

The SARS-CoV-2 Viral Load Quantitation Assay is a laboratory developed test that uses components of the TaqPath™ COVID-19 Combo Kit along with calibration standards to generate a calibration curve that converts Cq (Quantitation Cycle) values into viral genome copy number for quantitation of SARS-CoV-2 viral load in Nasopharyngeal Swab and Plasma samples.

Briefly, nucleic acids are extracted from these specimens using the MagMAX™ Viral/Pathogen Nucleic Acid Isolation Kit, via an automated process and are reverse-transcribed and amplified by polymerase chain reaction (RT-PCR). During RT-PCR, the probes anneal to three specific SARS-CoV-2 target sequences located between three unique forward and reverse primers for the ORF1ab, N Protein, and S Protein genes. During the extension phase of the PCR cycle, the 5′ nuclease activity of Taq polymerase degrades the probe, causing the reporter dye to separate from the quencher dye, generating a fluorescent signal. With each cycle, additional reporter dye molecules are cleaved from their respective probes, increasing the fluorescence intensity. Fluorescence intensity is monitored at each PCR cycle by the real-time PCR instrument.

The data are analyzed using the Design and Analysis software version 2.4.1 and converted to viral genome copies/mL of sample using a calibration curve generated from seven standards ranging from 500,000,000 (5E8) copies/mL to 500 (5E2) copies/mL.

Viral copy number is determined using the Cq data for the N Protein gene only.

### Results

Interim results of the salivary viral load analysis are displayed in FIG. 29, which plots the proportion of patients with available samples for analysis, at each timepoint, in whom the viral load in saliva is below the quantifiable limit of the assay 500 copies/mL. The panel entitled “no antivirals” displays the results for patients on standard of care alone [SoC] and bemcentinib in addition to standard of care [SoC+BEM], for those patients in whom another antiviral medication was not used during the course of their hospital treatment for COVID19. The panel entitled “antivirals” displays similar data, in the group of patients who received an antiviral medication, either remdesivir or favipiravir, for treatment of their COVID19 disease.

### Virologic Response in Standard of Care Alone

The results for SoC patients who did not receive an antiviral at baseline (n=22) are displayed in the panel entitled “no antivirals” of FIG. 29. The changing value here represents the natural history of viral clearance in patients treated with supportive therapy, but no medications with a specific antiviral effect. These data show from day 1, where just over 20% of patients have viral levels below 500 copies/ml in saliva, just under 40% at day 8, 60% at day 11, 80% and day 15 and around 90% by day 29. Note that detectable virus at these later timepoints may not necessarily indicate viable viral particles which remain infectious; infective capability would require a plaque assay methodology which is not readily available at the scale required for these clinical studies.

This pattern of viral clearance can be considered to be entirely due to the innate immune system clearing the virus, with the support of pharmacologic (immune modulators and oxygen) and non-pharmacologic support, including oxygenation and nursing care.

Viral Response in Standard of Care which Includes an Antiviral

These results are displayed in the panel entitled “antivirals” of FIG. 29; with proportions of just over 20% at day 1, through approximately 55% at day 8 and day 11, and 90% below LLOQ at day 15.

### Viral Response in Bemcentinib Arm in Presence or Absence of Antiviral

The bemcentinib results are displayed in each panel of FIG. 29 as indicated in the legend.

In the absence of another antiviral, at day 8 approximately 66% of bemcentinib treated patients (8 of 12) versus 37.5% (3 of 8) SoC, and at day 11, 100% of bemcentinib (3 of 3) versus 60% (3 of 5) SoC patients had salivary viral load below LLOQ.

In the presence of another antiviral, at day 8 approximately 82% of bemcentinib treated patients (9 of 11) versus 54% (7 of 13) SoC, and at day 11, 100% of bemcentinib (9 of 9) versus 56% (5 of 9) SoC patients had salivary viral load below LLOQ.

### SUMMARY

Taken together these results are supportive of an suppressive viral effect associated with treatment including bemcentinib, which is demonstrated by increased proportion of patients having undetectable virus in saliva at earlier timepoints, which is superimposed and independent of the effects of innate immune clearance and the use of concomitant antiviral pharmacologic therapies.

1.-64. (canceled)

65. A method for treating a virus infection in a subject, preventing or reducing transmission of a virus infection in a subject, or increasing viral clearance from a subject, the method comprising administering to the subject an effective amount of an inhibitor of AXL activity or expression (AXLi).

66. The method according to claim 65, wherein the virus infection is a coronavirus infection.

67. The method according to claim 65, wherein the virus infection is an alphaletovirus infection or an orthocoronavirus infection.

68. The method according to claim 66, wherein the coronavirus infection is selected from the group consisting of: an alphacoronavirus infection, a betacoronavirus infection, a gammacoronavirus infection, and a deltacoronavirus infection.

69. The method according to claim 65, wherein the virus infection is a SARS-CoV infection, a SARS-CoV-2 infection, or a MERS-CoV infection.

70. The method according to claim 65, wherein the AXLi is administered in combination with a second antiviral agent, an anti-inflammatory agent, or an immunosuppressive agent, or combinations thereof.

71. The method according to claim 70, wherein the second antiviral agent is:
(i) selected from the group consisting of: a protease inhibitor, a helicase inhibitor, and a cell entry inhibitor;
(ii) selected from the group consisting of: ribavirin and an interferon; or
(iii) remdesivir.

72. The method according to claim 70, wherein the anti-inflammatory agent is:
(i) a corticosteroid;
(ii) a glucocorticoid steroid; or
(iii) dexamethasone.

73. The method according to claim 70, wherein the immunosuppressive agent is:
(i) an IL-6 anatgonist; or
(ii) Tocilizumab.

74. The method according to claim 65, wherein the subject is human.

75. The method according to claim 65, wherein the subject has one or more comorbidities selected from: respiratory system disease, cardiovascular disease, diabetes, hypertension, cancer, or a suppressed immune system.

76. The method according to claim 65, wherein the subject is at least 60 years old.

77. The method according to claim 65, wherein the subject's C-reactive protein (CRP) level is at least 30 pg/mL.

78. The method according to claim 65, wherein the subject is selected for treatment on the basis of a CRP level of at least 30 pg/mL.

79. The method according to claim 65, wherein the AXLi is a compound of formula (I): 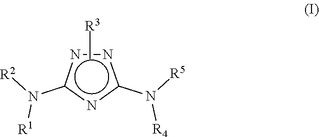
wherein:
R¹, R⁴ and R⁵ are each independently selected from the group consisting of hydrogen, alkyl, alkenyl, aryl, aralkyl, —C(O)R⁸, —C(O)N(R⁶)R⁷, and —C(═NR⁶)N(R⁶)R⁷;
R² and R³ are each independently a polycyclic heteroaryl containing more than 14 ring atoms optionally substituted by one or more substituents selected from the group consisting of oxo, thioxo, cyano, nitro, halo, haloalkyl, alkyl, optionally substituted cycloalkyl, optionally substituted cycloalkylalkyl, optionally substituted aryl, optionally substituted aralkyl, optionally substituted heteroaryl, optionally substituted heterocyclyl, —R⁹—OR⁸, —R⁹—O—R¹⁰—OR⁸, —R⁹—O—R¹⁰—O—R¹⁰—OR⁸, —R⁹—O—R¹⁰—CN, —R⁹—O—R¹⁰—C(O)OR⁸, —R⁹—O—R¹⁰—C(O)N(R⁶)R⁷, —R⁹—O—R¹⁰—S(O)ₚR⁸ (where p is 0, 1 or 2), —R⁹—O—R¹⁰—N(R⁶)R⁷, —R⁹—O—R¹⁰—C(NR¹¹)N(R¹¹)H, —R⁹—OC(O)—R⁸, —R⁹—N(R⁶)R⁷, —R⁹—C(O)R⁸, —R⁹—C(O)OR⁸, —R⁹—C(O)N(R⁶)R⁷, —R⁹—N(R⁶)C(O)OR⁸, —R⁹—N(R⁶)C(O)R⁸, —R⁹—N(R⁶)S(O)ₜR⁸ (where t is 1 or 2), —R⁹—S(O)ₜOR⁸ (where t is 1 or 2), —R⁹—S(O)ₚR⁸ (where p is 0, 1 or 2), and —R⁹—S(O)ₜN(R⁶)R⁷ (where t is 1 or 2); or
R² is a polycyclic heteroaryl containing more than 14 ring atoms as described above and R³ is selected from the group consisting of aryl and heteroaryl, where the aryl and the heteroaryl are each independently optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2); or
R³ is a polycyclic heteroaryl containing more than 14 ring atoms as described above, and R² is selected from the group consisting of aryl and heteroaryl, where the aryl and the heteroaryl are each independently optionally substituted by one or more substituents selected from the group consisting of alkyl, alkenyl, alkynyl, halo, haloalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹³—OR¹², —R¹³—OC(O)—R¹², —R¹³—O—R¹⁴—N(R¹²)₂, —R¹³—N(R¹²)—R¹⁴—N(R¹²)₂, —R¹³—N(R¹²)—R¹⁴—N(R¹²)₂, —R¹³—N(R¹²)₂, —R¹³—C(O)R¹², —R¹³—C(O)OR¹², —R¹³—C(O)N(R¹²)₂, —R¹³—C(O)N(R¹²)—R¹⁴—N(R¹²)R¹³, —R¹³—C(O)N(R¹²)—R¹⁴—OR¹², —R¹³—N(R¹²)C(O)OR¹², —R¹³—N(R¹²)C(O)R¹², —R¹³—N(R¹²)S(O)ₜR¹² (where t is 1 or 2), —R¹³—S(O)ₜOR¹² (where t is 1 or 2), —R¹³—S(O)ₚR¹² (where p is 0, 1 or 2), and —R¹³—S(O)ₜN(R¹²)₂ (where t is 1 or 2);
each R⁶ and R⁷ is independently selected from the group consisting of hydrogen, alkyl, alkenyl, alkynyl, haloalkyl, haloalkenyl, haloalkynyl, hydroxyalkyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or any R⁶ and R⁷, together with the common nitrogen to which they are both attached, form an optionally substituted N-heteroaryl or an optionally substituted N-heterocyclyl;
each R⁸ is independently selected from the group consisting of hydrogen, alkyl, alkenyl, alkynyl, haloalkyl, haloalkenyl, haloalkynyl, optionally substituted aryl, optionally substituted aralkyl, optionally substituted aralkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, and optionally substituted heteroarylalkynyl;
each R⁹ is independently selected from the group consisting of a direct bond, an optionally substituted straight or branched alkylene chain, an optionally substituted straight or branched alkenylene chain and an optionally substituted straight or branched alkynylene chain;
each R¹⁰ is independently selected from the group consisting of an optionally substituted straight or branched alkylene chain, an optionally substituted straight or branched alkenylene chain and an optionally substituted straight or branched alkynylene chain;
each R¹¹ is independently selected from the group consisting of hydrogen, alkyl, cyano, nitro and —OR⁸;
each R¹² is independently selected from the group consisting of hydrogen, alkyl, alkenyl, haloalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl, optionally substituted heteroarylalkyl, —R¹⁰—OR⁸, —R¹⁰—CN, —R¹⁰—NO₂, —R¹⁰—N(R⁸)₂, —R¹⁰—C(O)OR⁸ and —R¹⁰—C(O)N(R⁸)₂, or two R¹²'s, together with the common nitrogen to which they are both attached, form an optionally substituted N-heterocyclyl or an optionally substituted N-heteroaryl;
each R¹³ is independently selected from the group consisting of a direct bond, an optionally substituted straight or branched alkylene chain and an optionally substituted straight or branched alkenylene chain; and
each R¹⁴ is independently selected from the group consisting of an optionally substituted straight or branched alkylene chain and an optionally substituted straight or branched alkenylene chain; as an isolated stereoisomer or mixture thereof or as a tautomer or mixture thereof, or a pharmaceutically acceptable salt or N-oxide thereof.

80. The method according to claim 65, wherein the AXLi is:
(i) selected from the group consisting of:
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7-(R)-pyrrolidin-1-yl)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-(3-fluoro-4-(4-(pyrrolidin-1-yl)piperidin-1-yl)phenyl)-1H-1,2,4-triazole-3,5-diamine;
1-(6,7-dihydro-5H-benzo[6,7]cyclohepta[1,2-c]pyridazin-3-yl)-N⁵-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N⁵-(7-(S)-pyrrolidin-1-yl-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(acetamido)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(methoxycarbonyl)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4,4-difluoropiperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((methoxycarbonylmethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((2R)-2-(carboxy)pyrrolidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonyl)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxy)piperidin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-((carboxymethyl)(methyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(ethoxycarbonylmethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(4-(carboxymethyl)piperazin-1-yl)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-(7-(pyrrolidin-1-yl)-6,7,8,9-tetrahydro-5H-benzo[7]annulene-1-yl)-1H-1,2,4-triazole-3,5-diamine;
1-(6,7-dihydro-5H-benzo[6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7s)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-methylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((propyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((1-cyclopentylethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-propylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3,3-dimethylbut-2-yl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclohexylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((5-chlorothien-2-yl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2-carboxyphenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((3-bromophenyl)methyl)amino-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-pentylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((2,2-dimethylpropyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((cyclopentylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-((bicyclo[2.2.1]hept-2-en-5-ylmethyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(3-methylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(2-ethylbutylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(but-2-enylamino)-6,7,8,9-tetrahydro-5H-benzo[7]annulene-2-yl)-1H-1,2,4-triazole-3,5-diamine;
1-(6,7-dihydro-5H-benzo[6,7]cyclohepta[1,2-c]pyridazin-3-yl)-N³-((7S)-7-(butyl(but-2-enyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N⁵-((7S)-7-(t-butoxycarbonylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-amino-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dimethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(diethylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(dipropylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(cyclopropylmethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(di(3-methylbutyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclobutylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclohexylamino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-((methylethyl)amino)-6,7,8,9-tetrahydro-5H-benzo[7]annulene-2-yl)-1H-1,2,4-triazole-3,5-diamine;
1-(6,7-dihydro-5H-pyrido[2′,3′:6,7]cyclohepta[1,2-c]pyridazin-3-yl)-N³-((7S)-7-(cyclopentylamino)-6,7,8,9-tetrahydro-5H-benzo[7]annulene-2-yl)-1H-1,2,4-triazole-3,5-diamine; and
1-(6,7-dihydro-5H-pyrido[2′,3′:6,7]cyclohepta[1,2-c]pyridazin-3-yl)-N³-((7S)-7-(2-butylamino)-6,7,8,9-tetrahydro-5H-benzo[7]annulene-2-yl)-1H-1,2,4-triazole-3,5-diamine;
or pharmaceutically acceptable salts thereof,
(ii) selected from the group consisting of:
Dubermatinib (CAS No. 1341200-45-0; UNII 14D65TV20J);
Gilteritinib (CAS No. 1254053-43-4; UNII 66D92MGC8M);
Cabozantinib (CAS No. 849217-68-1; UNII 1C39JW444G);
SGI7079 (CAS No. 1239875-86-5);
Merestinib (CAS No. 1206799-15-6; UNII 5OGS5K699E);
Amuvatinib (CAS No. 850879-09-3; UNII SO9S6QZB4R);
Bosutinib (CAS No. 380843-75-4; UNII 5018V4AEZ0);
Sitravatinib (CAS No. 1123837-84-2; UNII CWG62Q1VTB);
XL092;
Glesatinib (CAS No. 936694-12-1; UNII 7Q290XD98N); and
foretinib (CAS No. 849217-64-7; UNII 81FH7VK1C4); or
(iii) 1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine, or a pharmaceutically acceptable salt thereof.

81. The method according to claim 65, wherein the AXLi is bemcentinib.

82. The method according to claim 65, wherein the AXLi is an antibody that:
(i) is selected from the group consisting of:
the 1613F12 antibody disclosed in US 2015/0037340 A1;
the 110D7 antibody disclosed in US 2016/0046725 A1;
the 1003A2 antibody disclosed in US 2016/0046725 A1;
the 1024G11 antibody disclosed in US 2016/0046725 A1;
the hu10G5 antibody disclosed in US 2021/0171643 A1; and
the YW327.6S2 antibody disclosed in US 2013/0243753 A1;
(ii) comprises the 6 CDRs having the sequences of SEQ ID Nos. 1 to 6;
(iii) comprises the 6 CDRs having the sequences of SEQ ID Nos. 7 to 12;
(iv) comprises:
a VH domain having the sequence of SEQ ID No. 13 and a VL domain having the sequence of SEQ ID NO.15;
a VH domain having the sequence of SEQ ID No. 13 and a VL domain having the sequence of SEQ ID NO.16;
a VH domain having the sequence of SEQ ID No. 14 and a VL domain having the sequence of SEQ ID NO.15; or
a VH domain having the sequence of SEQ ID No. 14 and a VL domain having the sequence of SEQ ID NO.16; or
(v) comprises all 6 of the CDRs comprised in:
a VH domain having the sequence of SEQ ID No. 13 and a VL domain having the sequence of SEQ ID NO.15;
a VH domain having the sequence of SEQ ID No. 13 and a VL domain having the sequence of SEQ ID NO.16;
a VH domain having the sequence of SEQ ID No. 14 and a VL domain having the sequence of SEQ I D NO.15; or
a VH domain having the sequence of SEQ ID No. 14 and a VL domain having the sequence of SEQ ID NO.16.

83. The method according to claim 65, wherein the AXLi is the antibody Tilvestamab.

84. A pharmaceutical composition comprising an AXLi according to claim 79 and a second anti-viral agent.
